(19) United States
(12) Patent Application Publication
(10) Pub. No.: US 2023/0407404 A1
(43) Pub. Date: Dec. 21, 2023

(54) METHODS AND COMPOSITIONS FOR ANALYZING IMMUNE INFILTRATION IN CANCER STROMA TO PREDICT CLINICAL OUTCOME

(71) Applicant: 10x Genomics, Inc., Pleasanton, CA (US)

(72) Inventors: Florian Baumgartner, Stockholm (SE); Valeria Giangarra, Rome (IT); Sarah Taylor, Dublin, CA (US); Yifeng Yin, Elk Grove, CA (US)

(21) Appl. No.: 18/037,670

(22) PCT Filed: Nov. 18, 2021
(86) PCT No.: PCT/US2021/059959

§ 371 Date: May 18, 2023

Related U.S. Application Data
(60) Provisional application No. 63/242,721, filed on Sep. 10, 2021.
(60) Provisional application No. 63/142,772, filed on Jan. 28, 2021.
(60) Provisional application No. 63/115,502, filed on Nov. 18, 2020.

(51) Int. Cl. C12Q 1/6886; G16H 50/20

(52) U.S. Cl. CPC C12Q 1/6886; G16H 50/20; C12Q 2600/118; C12Q 2600/158

(57) ABSTRACT

Provided herein are methods for analyzing immune cell infiltration in a cancer stromal region of a biological sample obtained from a subject using machine learning modules. For example, the methods may include (a) identifying a cancerous region or an analyte associated with the cancerous region in the biological sample; (b) identifying a stromal region or an analyte associated with the stromal region in the biological sample; (c) identifying one or more immune cells or an analyte associated with an immune cell in one or more locations in the biological sample; and (d) using (i) the identified cancerous and stromal regions or associated analytes thereof in the biological sample and (ii) the identified one or more immune cells or associated analytes thereof to analyze immune cell infiltration in the cancer stromal region of the biological sample obtained from the subject.

### CROSS-REFERENCE TO RELATED APPLICATIONS

This application claims the benefit of U.S. Provisional Application Ser. No. 63/115,502, filed Nov. 18, 2020, U.S. Provisional Application Ser. No. 63/142,772, filed Jan. 28, 2021, and U.S. Provisional Application Ser. No. 63/242,721, filed Sep. 10, 2021, the entire contents of each of which are incorporated by reference herein.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Understanding the regions of cellular and genetic heterogeneity could aid in development of individual treatments in patients that otherwise appear similar. At the same time, it is also important to identify immunological infiltrates, which are disparately expressed in certain areas of a tumor. Tumors can be heterogeneous (cellularly or genetically), with different regions within a tumor sample demonstrating different gene expression.

Tumor-infiltrating immune cells (e.g., tumor infiltrating lymphocytes, (“TILs”)) in a cancer tissue have been demonstrated to be a marker of response to immune-checkpoint therapy in several cancers and correlate with relapse status of the patient (See, e.g., Fares et al., American Society of Clinical Oncology Educational Book, 39, 147-164 (2019)). Pathologists have used standardized visual approaches to quantify TILs for therapy prediction. However, even with standardization efforts, successful visual identification of TIL estimation and detection of other immune cells in a biological sample remains a challenge. Moreover, the lack of precision limits the ability to evaluate more complex properties such as immune cell distribution patterns. Therefore, there remains a need to develop ways to identify and characterize tumor-infiltrating immune cells in a biological sample.

### SUMMARY

In one aspect, this disclosure features methods of analyzing immune cell infiltration in a cancer stromal region of a biological sample (e.g., sample obtained from a subject), including: (a) identifying a cancerous region or an analyte associated with the cancerous region in the biological sample; (b) identifying a stromal region or an analyte associated with the stromal region in the biological sample; (c) identifying one or more immune cells or an analyte associated with an immune cell in one or more locations in the biological sample; and (d) using (i) the identified cancerous and stromal regions or associated analytes thereof in the biological sample and (ii) the identified one or more immune cells or associated analytes thereof to analyze immune cell infiltration in the cancer stromal region of the biological sample (e.g., sample obtained from the subject).

In some embodiments, the identifying the cancerous region, the identifying the stromal region, and/or the identifying immune cells includes: (a) generating a dataset from the biological sample, wherein the dataset includes one or more of: (i) analyte data for a plurality of analytes captured from a plurality of spatial locations in the biological sample; (ii) image data including images of the plurality of spatial locations of the biological sample; and (iii) registration data linking the analyte data to the image data; and (b) using the dataset to identify the cancerous region, the stromal region, and/or the immune cells in the biological sample.

In some embodiments, (b) includes providing the dataset to a trained machine learning module, wherein the trained machine learning module is trained at least in part from training data including reference analyte datasets from one or more reference samples, wherein the one or more reference samples include (1) one or more reference cancerous regions, (2) one or more reference stromal regions, and (3) one or more reference immune cells.

In some embodiments, the abundance of immune cells is determined via the trained machine learning module.

In some embodiments, the cancerous region includes one or more of a benign tumor, a pre-metastatic tumor, a malignant tumor, and one or more inflammatory cells.

In some embodiments, the stromal region includes one or more of connective tissue, blood vessels, and inflammatory cells.

In some embodiments, the method further includes permeabilizing the biological sample.

In some embodiments, the analyte associated with the cancerous region, an analyte associated with the stromal region, and/or an analyte associated with an immune cell is a nucleic acid. In some embodiments, the nucleic acid is RNA. In some embodiments, the RNA is an mRNA.

In some embodiments, the analyte associated with the cancerous region, the analyte associated with the stromal region, and/or the analyte associated with an immune cell is detected by the steps including: contacting the biological sample with a substrate including a plurality of capture probes, wherein a capture probe of the plurality of capture probes includes a spatial barcode and a capture domain; hybridizing the analyte associated with the cancerous region, the analyte associated with the stromal region, and/or the analyte associated with an immune cell to the capture probe; and determining (i) all or a part of a sequence corresponding to the analyte associated with the cancerous region, the analyte associated with the stromal region, and/or the analyte associated with an immune cell, or a complement thereof, and (ii) all or a part of a sequence corresponding to the spatial barcode, or a complement thereof, and using the determined sequence of (i) and (ii) to identify the abundance and/or spatial location of the analyte associated with the cancerous region, the analyte associated with the stromal region, and/or the analyte associated with an immune cell, or a complement thereof in the biological sample.

In some embodiments, the determining step includes sequencing.

In some embodiments, the analyte associated with the cancerous region, an analyte associated with the stromal region, and/or an analyte associated with an immune cell is a protein. In some embodiments, the analyte associated with the cancerous region, the analyte associated with the stromal region, and/or the analyte associated with an immune cell is detected by the steps including: attaching the biological sample with a plurality of analyte capture agents, wherein an analyte capture agent of the plurality of analyte capture agents includes: (i) an analyte binding moiety that binds specifically to the analyte associated with the cancerous region, the analyte associated with the stromal region, and/or the analyte associated with an immune cell; (ii) an analyte binding moiety barcode; and (iii) an analyte capture sequence, wherein the analyte capture sequence binds specifically to a capture domain; contacting the biological sample with a substrate, wherein the substrate includes a plurality of capture probes, wherein a capture probe of the plurality of capture probes includes (i) the capture domain and (ii) a spatial barcode; hybridizing the analyte associated with the cancerous region, the analyte associated with the stromal region, and/or the analyte associated with an immune cell to the capture probe; and determining (i) all or a part of a sequence corresponding to the analyte associated with the cancerous region, the analyte associated with the stromal region, and/or the analyte associated with an immune cell, or a complement thereof, and (ii) all or a part of a sequence corresponding to the spatial barcode, or a complement thereof, and using the determined sequence of (i) and (ii) to identify the abundance and/or spatial location of the analyte associated with the cancerous region, the analyte associated with the stromal region, and/or the analyte associated with an immune cell, or a complement thereof in the biological sample.

In some embodiments, the determining step includes: sequencing (i) all or a part of a sequence corresponding to the analyte associated with the cancerous region, the analyte associated with the stromal region, and/or the analyte associated with an immune cell, or a complement thereof, and (ii) all or a part of a sequence corresponding to the spatial barcode, or a complement thereof, and using the determined sequence of (i) and (ii) to identify the abundance and/or spatial location of the analyte associated with the cancerous region, the analyte associated with the stromal region, and/or the analyte associated with an immune cell, or a complement thereof in the biological sample.

In some embodiments, the analyte binding moiety is an antibody or antigen-binding fragment thereof, a cell surface receptor binding molecule, a receptor ligand, a small molecule, a T-cell receptor engager, a B-cell receptor engager, a pro-body, an aptamer, a monobody, an affimer, or a darpin.

In some embodiments, the analyte associated with the cancerous region, the analyte associated with the stromal region, and/or the analyte associated with an immune cell is detected using in situ sequencing.

In some embodiments, the analyte associated with the cancerous region, the analyte associated with the stromal region, and/or the analyte associated with an immune cell is detected using an antibody.

In some embodiments, the method further includes contacting the biological sample with one or more stains. In some embodiments, the one or more stains includes hematoxylin and eosin. In some embodiments, the one or more stains include one or more optical labels. In some embodiments, the one or more optical labels are selected from the group consisting of: fluorescent, radioactive, chemiluminescent, calorimetric, or colorimetric labels.

In some embodiments, the method further includes identifying one or more cancerous regions in the biological sample using the one or more stains of the biological sample. In some embodiments, the stain is specific to a cancer marker. In some instances, the cancer marker is pancytokeratin (Pan-CK or PAN-CK).

In some embodiments, the method further includes identifying one or more stromal regions within the one or more cancerous regions using the one or more stains of the biological sample. In some embodiments, the stain is specific to a stromal marker. In some instances, the cancer marker is CD45. In some embodiments, the image data is generated using a method including obtaining an image of the biological sample. In some embodiments, the method further includes registering the image data to a spatial location. In some embodiments, the method further includes identifying (1) the one or more cancerous regions and/or (2) the one or more stromal regions based on the image data. In some embodiments, the method further includes identifying the one or more immune cells based on the image data.

In some embodiments, the method further includes identifying the one or more cancerous regions via the trained machine learning module. In some embodiments, the method further includes identifying the one or more stromal regions via the trained machine learning module. In some embodiments, the method further includes identifying the one or more immune cells via the trained machine learning module.

In some embodiments, the analysis of immune cell infiltration in the cancer stromal region of the biological sample includes determining abundance of immune cells in the cancer stromal region in the biological sample.

In some embodiments, identifying the one or more cancer regions includes: (i) obtaining an image and registering the image data to the spatial location, (ii) using the spatial location of the determined sequences, or (iii) obtaining an image and registering the image data to the spatial location, and using the spatial location of the determined sequences; identifying the one or more stromal regions includes: (i) obtaining an image and registering the image data to the spatial location, (ii) using the spatial location of the determined sequences, or (iii) obtaining an image and registering the image data to the spatial location, and using the spatial location of the determined sequences; and identifying the one or more immune cells or associated analytes thereof in one or more locations in the biological sample includes: (i) obtaining an image and registering the image data to the spatial location, (ii) using the spatial location of the determined sequences, or (iii) obtaining an image and registering the image data to the spatial location, and using the spatial location of the determined sequences.

In some embodiments, the abundance of immune cells in the cancer stromal region is determined as a percentage of cells in the cancer stroma area that are immune cells or a percentage of area of the cancer stroma that is occupied by immune cells.

In some embodiments, the abundance of immune cells in the cancer stromal region is determined using the spatial location of the determined sequence of the one or more cancerous regions, one or more stromal regions, and one or more immune cells.

In some embodiments, the using the spatial location of the determined sequences includes determining the sequence using in situ sequencing. In some embodiments, the abundance of immune cells in the cancer stromal region is determined using segmenting and (i) obtaining an image and registering the image data to the spatial location, (ii) using the spatial location of the determined sequences, or (iii) obtaining an image and registering the image data to the spatial location, and using the spatial location of the determined sequences.

In some embodiments, the determining includes: (a) identifying the amount of genes associated with immune infiltrating cells compared to known housekeepers normalized by number of cells per spatial location; (b) identifying the ratio of one or more tumor infiltrating lymphocytes (TILs) to one or more tumor infiltrating B cells (TIBs); and/or (c) calculating the abundance of tumor infiltrating immune cells in the biological sample based on the percentage of spatial locations including analytes associated with an immune infiltrating cells.

In some embodiments, the identification of the one or more immune cells includes segmenting immune cells from the image data.

In some embodiments, the further includes determining a cancer prognosis based on the immune infiltration.

In some embodiments, the further includes scoring or determining the severity of the cancer in the subject based on the immune infiltration score.

In some embodiments, the determining includes identifying the ratio of one or more tumor infiltrating lymphocytes (TILs) to one or more tumor infiltrating B cells (TIBs) or one or more tumor infiltrating T cells to one or more tumor infiltrating B cells (TIBs).

In some embodiments, the further includes administering a therapeutic treatment (e.g., to a subject), wherein the therapeutic treatment includes surgery, chemotherapeutic agents, growth inhibitory agents, cytotoxic agents, agents used in radiation therapy, anti-angiogenesis agents, cancer immunotherapeutic agents, apoptotic agents, antitubulin agents, or a combination thereof.

In some embodiments, the biological sample is obtained from a biopsy (e.g., from a subject). In some embodiments, the biological sample is obtained from a surgical excision (e.g., from a subject). In some embodiments, the biological sample is collected during an endoscopy or colonoscopy (e.g., from a subject). In some embodiments, the biological sample is a tissue section. In some embodiments, the biological sample is a tissue section on a slide. In some embodiments, the biological sample is a formalin-fixed, paraffin-embedded (FFPE) sample, a frozen sample, or a fresh sample. In some embodiments, the biological sample is an FFPE sample.

In some embodiments, the immune cells are selected from a B cell, a T cell, an NK cell, a monocyte, a macrophage, a neutrophil, a granulocyte, an innate lymphoid cell, or a dendritic cell, or a combination thereof.

In some embodiments, the analyte associated with the cancerous region is selected from an analyte from the AKT pathway, an analyte from the JAK-STAT pathway, and an analyte from the Notch pathway, or a combination thereof.

In some embodiments, the analyte associated with the cancerous region is selected from SCGB2A1, MK167, BRCA1, BRCA2, PIKCD, CALML6, MYC, TP53, PALB2, RAD51, and MSH2, or a combination thereof. In some instances, the analyte associated with the cancerous region is selected from SCGB2A1, MKI67, BRCA1, BRCA2, PIK3CD, and CALML6, or a combination thereof. In some instances, the analyte associated with the cancerous region is selected from PRKCI, VTCN1, MECOM, TOP2A, SHDH, XPO1, TFRC, FUT8, SOX17, PBX1, EIF42, and WTT, or a combination thereof. In some instances, the analyte associated with the cancerous region is selected from VTCN1, MECOM, TOP2A, XPO1, FUT8, SOX17, PBX1, EIF42, and WTT, or a combination thereof. In some instances, the analyte associated with the cancerous region is TOP2A. In some instances, the analyte associated with the cancerous region is XPO1. Non-limiting examples of analytes disclosed in this paragraph can also include byproducts, precursors, and degradation products of such analytes thereof, and any combination of such analytes and byproducts, precursors, and degradation products thereof.

In some embodiments, the analyte associated with the stromal region is selected from VIM, EPCAM, FAP, and CDH1. In some embodiments, the analyte associated with the stromal region is selected from FAP, VCAN, ACTA2, and PDGFRB. In some embodiments, the analyte associated with an immune cell is selected from BLK, CD19, FCRL2, MS4A1, KIAA0125, TNFRSF17, TCL1A, SPIB, PNOC, PTRPC, PRF1, GZMA, GZMB, NKG7, GZMH, KLRK1, KLRB1, KLRD1, CTSW, GNLY, CCL13, CD209, HSD11B1, LAG3, CD244, EOMES, PTGER4, CD68, CD84, CD163, MS4A4A, TPSB2, TPSAB1, CPA3, MS4A2, HDC, FPR1, SIGLEC5, CSF3R, FCAR, FCGR3B, CEACAM3, S100A12, KIR2DL3, KIR3DL1, KIR3DL2, IL21R, XCL1, XCL2, NCR1, CD6, CD3D, CD3E, SH2D1A, TRAT1, CD3G, TBX21, FOXP3, CD8A, CD8B, CD79A, CD79B, CD4, IGHA1, IGHG2, JCHAIN, IGKC, CD27, CD38, CD16, IL17RB, FANK1, CTLA4, MSR1, MRC1, NKG7, FCN1, TIGIT/LAG3. Non-limiting examples of analytes described in this paragraph can also include byproducts, precursors, and degradation products of such analytes thereof, and any combination of such analytes and byproducts, precursors, and degradation products thereof.

In some embodiments, the one or more immune cells is selected from: (i) a CD3⁺ and CD4⁺ T cell; (ii) a CD3⁺ and CD8⁺ T cell; (iii) a regulatory T cell including one or more of: CD4, Foxp3, IL17RB, CTLA4, FANK1, HAVCR1, CD25, CTLA-4, GITR, LAG-3, and CD127; (iv) a TH1 cell including one or more of: CD4, CD3D, S100A4, IL7R, and IFNG; (v) a TH2 cell including one or more of: CD4, IL7R, ICOS, CTLA4, TNFRSF4, and TNFRS18; (vi) a TH17 cell including one or more of: CD4, CD3D, IL17A, GZMA, and S100A4; (vii) a cytotoxic T cell including one or more of: CD8, CD3D, S100A4, IFNG, GZMB, GZMA, and IL2RB; (viii) a plasma cell including: one or more JCHAIN, MZB1, IGHA1, IGHG1, and IGKC; (ix) a monocyte including CD14⁺ CD16⁻; (x) a monocyte including CD14⁻ CD16⁺; and (xi) a natural killer cell including NKG7.

In some embodiments, the immune infiltrating cells is a tumor infiltrating B cell (TIB). In some embodiments, the TIB is selected from: (i) a plasma cell including one or more of: MZB1, IGLL5, IGHA1, IGHG1, JCHAIN, IGKC, IGHA2, IGLC2, IGLV3-1, and IGLV2-14; (ii) an Ig⁺ B cells including one or more of: IGHV3-74, SOCS3, JCHAIN, and SPARC; (iii) an activated B cell including: CD79B, HMGB2, HMGB1, HMGN1, and RGS13; (iv) a B cell including one or more of: MEF2B, RGS13, and MS4A1; and (v) a B cell including CD79A and CD79B. In some embodiments, the immune infiltrating cells is a plasma cell including one or more of: MZB1, IGLL5, IGHA1, IGHG1, JCHAIN, IGKC, IGHA2, IGLC2, IGLV3-1, and IGLV2-14.

In another aspect, this disclosure features methods of determining immune cell infiltration in a biological sample including one or more cancerous regions and one or more stromal regions in a subject including: (a) generating a dataset from the biological sample obtained from the subject, wherein the dataset includes: (i) analyte data for a plurality of analytes captured from a plurality of spatial locations of the biological sample, wherein an analyte in the plurality of analytes is an analyte associated with the cancerous region, an analyte associated with the stromal region, and/or an analyte associated with an immune cell; (b) providing the dataset to a trained machine learning module, wherein the trained machine learning module includes reference analyte datasets from one or more reference samples, wherein the one or more reference samples includes (i) a cancerous region from one or more cancerous regions, (2) a stromal region from one or more stromal regions, and (3) an immune cells from one or more immune cells; and (c) determining, via the trained machine learning module, the immune cell infiltration in the biological sample obtained from the subject.

In another aspect, this disclosure features methods of determining immune cell infiltration in a biological sample including one or more cancerous regions and one or more stromal regions including: (a) generating a dataset from the biological sample obtained from a subject, wherein the dataset includes: (i) analyte data for a plurality of analytes captured from a plurality of spatial locations of the biological sample, wherein an analyte in the plurality of analytes is an analyte associated with the cancerous region, an analyte associated with the stromal region, and/or an analyte associated with an immune cell; (ii) image data including images of the plurality of spatial locations of the biological sample; and (iii) registration data linking the analyte data to the image data; (b) providing the dataset to a trained machine learning module, wherein the trained machine learning module includes reference analyte datasets from one or more reference samples, wherein the one or more reference samples includes (i) a cancerous region from one or more cancerous regions, (2) a stromal region from one or more stromal regions, and (3) an immune cells from one or more immune cells; and (c) determining, via the trained machine learning module, the immune cell infiltration in the biological sample.

In some embodiments, the trained machine learning module is at least one of a supervised learning module, a semisupervised learning module, an unsupervised learning module, a regression analysis module, a reinforcement learning module, a self-learning module, a feature learning module, a sparse dictionary learning module, an anomaly detection module, a generative adversarial network, a convolutional neural network, or an association rules module.

In some embodiments, generating the dataset includes: contacting a biological sample (e.g., from the subject having cancer) with a substrate including a plurality of capture probes, wherein the biological sample includes (1) one or more cancerous regions, (2) one or more stromal regions, and (3) one or more tumor infiltrating immune cells, and wherein a capture probe of the plurality of capture probes includes a spatial barcode and a capture domain; attaching an analyte from the biological sample to the capture probe; determining (i) all or a part of a sequence corresponding to the analyte, or a complement thereof, and (ii) all or a part of a sequence corresponding to the spatial barcode, or a complement thereof, and using the determined sequence of (i) and (ii) to identify the spatial location and abundance of the analyte in the biological sample; and identifying a spatial location as being part of a cluster based on the determined sequences corresponding to the analytes at the spatial location and using the clusters to analyze immune cell infiltration in the cancer stroma of the subject having cancer.

In some embodiments, a cluster one or more immune cells is identified using one of the methods selected from: nonlinear dimensionality reduction, t-distributed stochastic neighbor embedding (t-SNE), global t-distributed stochastic neighbor embedding (g-SNE), and uniform manifold approximation and projection (UMAP).

In some embodiments, generating the dataset includes: attaching the biological sample with a plurality of analyte capture agents, wherein an analyte capture agent of the plurality of analyte capture agents includes: (i) an analyte binding moiety that binds specifically to the analyte associated with the cancerous region, the analyte associated with the stromal region, and/or the analyte associated with an immune cell; (ii) an analyte binding moiety barcode; and (iii) an analyte capture sequence, wherein the analyte capture sequence binds specifically to a capture domain; contacting the biological sample with a substrate, wherein the substrate includes a plurality of capture probes, wherein a capture probe of the plurality of capture probes includes (i) the capture domain and (ii) a spatial barcode; hybridizing the analyte associated with the cancerous region, the analyte associated with the stromal region, and/or the analyte associated with an immune cell to the capture probe; and determining (i) all or a part of a sequence corresponding to the analyte associated with the cancerous region, the analyte associated with the stromal region, and/or the analyte associated with an immune cell, or a complement thereof, and (ii) all or a part of a sequence corresponding to the spatial barcode, or a complement thereof, and using the determined sequence of (i) and (ii) to identify the abundance and/or spatial location of the analyte associated with the cancerous region, the analyte associated with the stromal region, and/or the analyte associated with an immune cell, or a complement thereof biological sample.

In some embodiments, the analyte data is generated using in situ sequencing.

In another aspect, this disclosure features a kit including: (a) a histology stain; (b) a substrate including a plurality of capture probe, wherein an capture probe of the plurality of capture probes includes a capture domain; and (c) instructions for performing any of the methods described herein.

In another aspect, this disclosure features a kit including: (a) an antibody that specifically binds to an antigen on an infiltrating immune cell; (b) a substrate including a plurality of capture probe, wherein an capture probe of the plurality of capture probes includes a capture domain; and (f) instructions for performing any of the methods described herein.

In another aspect, this disclosure features a kit including: (a) an antibody that specifically binds to an antigen on an infiltrating immune cell; (b) a second antibody that specifically binds to an antigen on a stromal cell; (c) a substrate including a plurality of capture probe, wherein an capture probe of the plurality of capture probes includes a capture domain; and (d) instructions for performing any of the methods described herein.

In another aspect, this disclosure features computer implemented methods, where the methods include: (a) generating a dataset of a plurality of biological samples, wherein the dataset includes, for each biological sample of the plurality of biological samples: (i) analyte data for a plurality of analytes captured at a plurality of spatial locations of a reference biological sample; (ii) image data of the reference biological sample; and (iii) registration data of the imaged data linking to the analyte data according to the spatial locations of the reference biological sample; wherein the reference biological sample includes (1) one or more cancerous regions in the reference biological sample, (2) one or more stromal regions within the one or more cancerous regions, and (3) a plurality of tumor infiltrating lymphocytes (TILs); (b) training a machine learning module with the dataset, thereby generating a trained machine learning module; and (c) determining immune cell infiltration in a biological sample via the trained machine learning module.

In another aspect, this disclosure features systems, where the systems include: (a) a storage element operable to store a dataset of a plurality of biological samples, wherein the dataset includes, for each biological sample: analyte data for a plurality of analytes captured at a plurality of spatial locations of a reference biological sample; image data of the biological sample; and registration data of the imaged data linking to the analyte data according to the spatial locations of the reference biological sample; wherein the biological sample includes (1) one or more cancerous regions in the reference biological sample, (2) one or more stromal regions within the one or more cancerous regions, and (3) the a plurality of tumor infiltrating lymphocytes (TILs); and (b) a processor operable to process the dataset through a machine learning module to train the machine learning module, to determine immune cell infiltration in a biological sample.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, including mixtures thereof “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe.

FIG. 2 is a schematic diagram of an exemplary analyte capture agent.

FIG. 3 is a schematic diagram depicting an exemplary interaction between a feature-immobilized capture probe 324 and an analyte capture agent 326.

FIGS. 4A-4C are schematics illustrating how streptavidin cell tags can be utilized in an array-based system to produce a spatially-barcoded cell or cellular contents.

FIG. 5 is a block diagram of an exemplary system for machine learning patterns in a biological sample.

FIG. 6 is a block diagram illustrating registration of image data to analyte data obtained from a capture area.

FIG. 7 is a flowchart of an exemplary process of the system of FIG. 5.

FIG. 8 shows immunofluorescence staining of a tissue section of an ovarian adenocarcinoma showing (i) merged image, (ii) pan-cytokeratin (Pan-CK), and (iii) CD45 (top panels) and a gene expression heat map of (i) all genes, (ii) MKi67, and (iii) PTPRC in the tissue section (bottom panels).

FIG. 9 shows an immunofluorescence stain for a Pan-CK antibody (left panel) and a gene expression heat map of a subset of cancer markers (right panel).

FIGS. 10A-10D show gene expression heat maps and correlation plots for targeted panels. FIGS. 10B-10D further provide correlation plots for the targeted panels.

FIG. 11A shows a violin plot of gene expression in each of eight different clusters for B cell markers CD19, CD79A, and CD79B.

FIG. 11B shows a gene expression heat map for the B cell markers in FIG. 11A (left panel) and an overlay of the gene expression heat map (left panel) and immunofluorescence staining for CD45 and Pan-CK (right panel).

FIG. 11C shows a violin plot of gene expression in each of eight different clusters for T cell markers CD3D, CD3E, CD4, and CD8A.

FIG. 11D shows a gene expression heat map for the T cell markers in FIG. 11C.

FIG. 12A shows an overlay of a gene expression heat map for T cell markers CD4, CD3E, and CD3D and immunofluorescence staining for CD45 and Pan-CK.

FIG. 12B shows an overlay of a gene expression heat map for T cell markers CD4 and CD14, and immunofluorescence staining for CD45 and Pan-CK.

FIG. 13 shows an overlay of a gene expression heat map for monocyte marker CD14.

FIG. 14 shows a gene expression heat map for CD4 (upper left panel), a gene expression heat map for all genes detected in the sample (upper right panel), and a violin plot of gene expression (Log 2 Expression) in each of eight different clusters for CD4 (lower panel).

FIG. 15 shows a gene expression heat map for CD8A (upper left panel), a gene expression heat map for all genes detected in the sample (upper right panel), and a violin plot of gene expression in each of eight different clusters for CD8 (lower panel).

FIG. 16A shows a gene expression heat map for plasma B cell markers: CD79A, CD79B, CD38, CD27, MZB1, IGHA1, IGHG1, JCHAIN, and IGKC.

FIG. 16B shows a gene expression heat map for JCHAIN.

FIG. 16C shows an immunofluorescence stain for CD45.

FIG. 17A shows a gene expression heat map for monocyte marker CD14.

FIG. 17B shows a gene expression heat map for monocyte marker CD16 (FCGR3A).

FIG. 17C shows an overlay of a gene expression heat map and immunofluorescence staining for CD45, DAPI, and Pan-CK.

FIG. 18 shows a gene expression heat map for T regulatory (Treg) cell markers FOXP3, IL17RB, CTLA4, FANK1, and CD4 (left panel) and a gene expression heat map for tumor-associated macrophage markers CD163, MSR1, and MRC1 (right panel).

FIG. 19 shows a gene expression heat map for Natural Killer (NK) marker NKG7 in a ovarian tumor sample (left panel), an overlay of a gene expression heat map for NKG7 and immunofluorescence staining for CD45 and Pan-CK in the ovarian tumor sample (center panel), and a gene expression heat map for Natural Killer (NK) marker NKG7 in a breast tumor IDC sample (right panel).

FIG. 20 shows an overlay of a gene expression heat map for CD4 and immunofluorescence staining for CD45 (left panel), an overlay of a gene expression heat map for CD8A and immunofluorescence staining for CD45 (center panel), and an overlay of a gene expression heat map for TIGIT/LAG3 and immunofluorescence staining for CD45 (right panel).

FIG. 21 shows a gene expression heat map for CD3E and CD4 (left panel) and a gene expression heat map for CD4 and CD14 (right panel).

FIG. 22A shows a violin plot of gene expression in each of eight different clusters for fibroblast activation protein alpha (FAP).

FIG. 22B shows a gene expression heat map for FAP.

FIG. 22C shows a violin plot of gene expression in each of eight different clusters for cadherin 1 (CDH1).

FIG. 22D shows an overlay of a gene expression heat map for the CDH1 and immunofluorescence stain for CD45.

FIG. 23A shows a violin plot of gene expression in each of eight different clusters for vimentin (VIM).

FIG. 23B shows an overlay of the gene expression heat map for VIM and immunofluorescence staining for CD45.

FIG. 23C shows a violin plot of gene expression in each of eight different clusters for epithelial cell adhesion molecule (EPCAM).

FIG. 23D shows an overlay of the gene expression heat map for EPCAM and immunofluorescence staining for CD45.

FIG. 24A shows a violin plot of gene expression in each of eight different clusters for ovarian cancer genes BRCA1, BRCA2, MYC, TP53, PALB2, RAD51, and MSH2.

FIG. 24B shows an overlay of the gene expression heat map for ovarian cancer genes from FIG. 24A and immunofluorescence staining for CD45.

FIG. 24C shows a violin plot of gene expression in each of eight different clusters for mutS homolog 2 (MSH2).

FIG. 24D shows an overlay of the gene expression heat map for MSH2 and immunofluorescence staining for CD45 (left panel) and an overlay of the gene expression heat map for MSH2 and immunofluorescence staining for Pan-CK (right panel).

FIG. 25A shows a violin plot of gene expression in each of eight different clusters for BRCA1.

FIG. 25B shows an overlay of the gene expression heat map for BRCA1 and immunofluorescence staining for CD45.

FIG. 25C shows a violin plot of gene expression in each of eight different clusters for BRCA2.

FIG. 25D shows an overlay of the gene expression heat map for BRCA2 and immunofluorescence staining for CD45.

FIG. 26 shows gene-expression heat maps for PI3K-AKT signaling components, Jak-STAT signaling components, and Notch signaling components and immunofluorescence staining for Pan-CK.

FIG. 27 shows gene-expression heat maps for nucleus components, phosphoproteins, polymorphisms components, and cellular process and an immunofluorescence staining for Pan-CK.

FIGS. 28A and 28B show overlapping tissue plot with spots using k-means unsupervised clustering (FIG. 28A) and immunofluorescence staining of Pan-CK and CD45 (FIG. 28B).

FIG. 28C shows a heat map of most dysregulated genes in the tumor (co-localized with Pan-CK) and stromal clusters (co-localized with CD45).

FIG. 28D shows a tissue plot providing colocalized detection of Pan-CK and CD45 with 9 clusters.

FIG. 28E shows a heat map of the most dysregulated genes in 9 clusters.

FIG. 29A shows tissue gene expression of a subset of cancer marker genes (SCGB2A1, MKI67, BRCA1, BRCA2, PIK3CD, and CALML6) with the tumor (Pan-CK-expressing) compartment.

FIG. 29B shows a violin plot of expression of a subset of cancer marker genes (SCGB2A1, MKI67, BRCA1, BRCA2, PIK3CD, and CALML6) with the tumor or stromal compartment.

FIG. 30A shows tissue gene expression of a subset of stromal marker genes (FAP, VCAN, ACTA2, and PDGFRB) with the stromal (CD45-expressing) compartment.

FIG. 30B shows a violin plot of expression of a subset of stromal marker genes (FAP, VCAN, ACTA2, and PDGFRB) with the tumor or stromal compartment.

FIG. 31A shows Pan-CK and CD45 expression in a tissue sample.

FIGS. 31B-31K shows tissue co-localized expression of Pan-CK and CD45 with expression of T cells CD3D, CD3E, CD4, CD8A, and CD247 (FIG. 31B), CD4 T cells (FIG. 31C), CD8A T Cells (FIG. 31D), Treg cells (FIG. 31E), B cells (FIG. 31F), plasma B cells (FIG. 31G), NK cells (FIG. 31H), CD14 monocytes (FIG. 31I), CD16 monocytes (FIG. 31J), and TAMs (FIG. 31K).

FIG. 32A shows immunofluorescence staining of Pan-CK, CD45, and DAPI in an ovarian tissue sample.

FIG. 32B shows tissue gene expression of clusters of cancer and stromal compartments in the tissue sample of FIG. 32A. Cluster 1 overlaps predominantly with Pan-CK tumor sections while Cluster 4 overlaps predominantly with CD45 stromal tissue sections. In Cluster 1, PRKCI, VTCN1, MECOM, TOP2A, SHDH, XPO1, TFRC, FUT8, SOX17, PBX1, E1F42, and WT1 were upregulated.

FIG. 32C shows gene expression for TOP2A in the tissue sample of FIG. 32A.

FIG. 32D shows gene expression for XPO1 in the tissue sample of FIG. 32A.

### DETAILED DESCRIPTION

### I. Introduction

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the analyte. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or 0-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

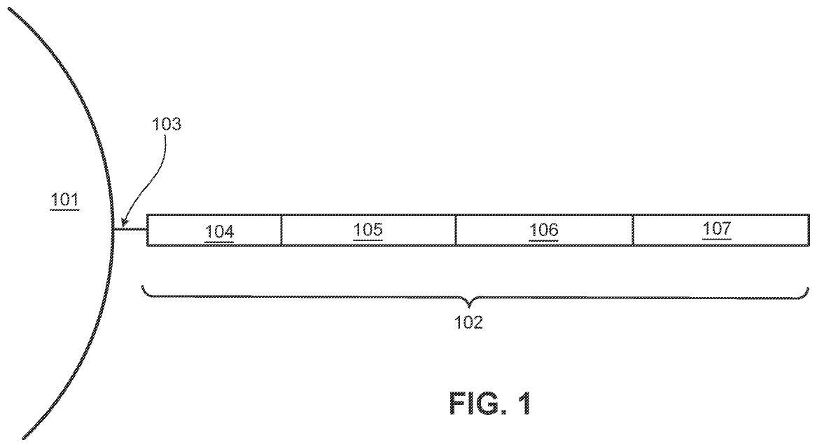

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises an additional functional sequence that can be located, e.g., between spatial barcode 105 and UMI sequence 106, between UMI sequence 106 and capture domain 107, or following capture domain 107.

The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

In some instances, the capture probe is a cleavable capture probe, wherein the cleaved capture probe can enter into a non-permeabilized cell and bind to analytes within the sample. The capture probe contains a cleavage domain, a cell penetrating peptide, a reporter molecule, and a disulfide bond (—S—S—).

In some instances, the disclosure provides a multiplexed spatially-barcoded feature. For instance, a feature can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode. One type of capture probe associated with the feature includes the spatial barcode in combination with a poly(T) capture domain, designed to capture mRNA target analytes. A second type of capture probe associated with the feature includes the spatial barcode in combination with a random N-mer capture domain for gDNA analysis. A third type of capture probe associated with the feature includes the spatial barcode in combination with a capture domain complementary to a capture handle sequence of an analyte capture agent of interest. A fourth type of capture probe associated with the feature includes the spatial barcode in combination with a capture domain that can specifically bind a nucleic acid molecule that can function in a CRISPR assay (e.g., CRISPR/Cas9). The disclosure can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof, (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

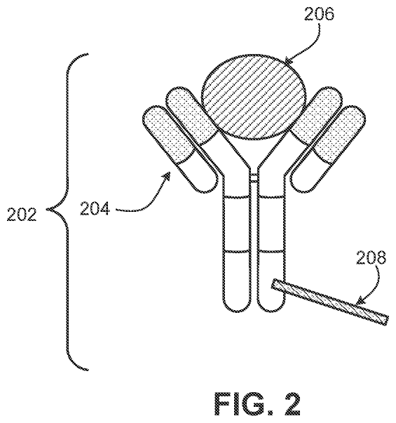

FIG. 2 is a schematic diagram of an exemplary analyte capture agent 202 comprised of an analyte-binding moiety 204 and an analyte-binding moiety barcode domain 208. The exemplary analyte-binding moiety 204 is a molecule capable of binding to an analyte 206 and the analyte capture agent is capable of interacting with a spatially-barcoded capture probe. The analyte-binding moiety can bind to the analyte 206 with high affinity and/or with high specificity. The analyte capture agent can include an analyte-binding moiety barcode domain 208, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 204 can include a polypeptide and/or an aptamer. The analyte-binding moiety 204 can include an antibody or antibody fragment (e.g., an antigen-binding fragment).

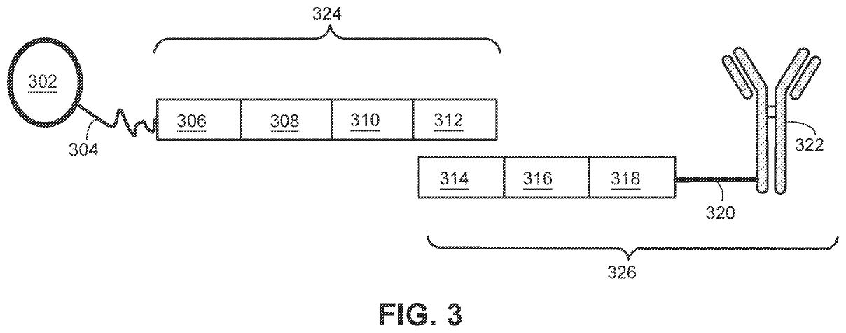

FIG. 3 is a schematic diagram depicting an exemplary interaction between a feature-immobilized capture probe 324 and an analyte capture agent 326. The feature-immobilized capture probe 324 can include a spatial barcode 308 as well as functional sequences 306 and UMI 310, as described elsewhere herein. The capture probe can also include a capture domain 312 that is capable of binding to an analyte capture agent 326. The analyte capture agent 326 can include a functional sequence 318, analyte binding moiety barcode 516, and a capture handle sequence 314 that is capable of binding to the capture domain 312 of the capture probe 324. The analyte capture agent can also include a linker 320 that allows the capture agent barcode domain 316 to couple to the analyte binding moiety 322.

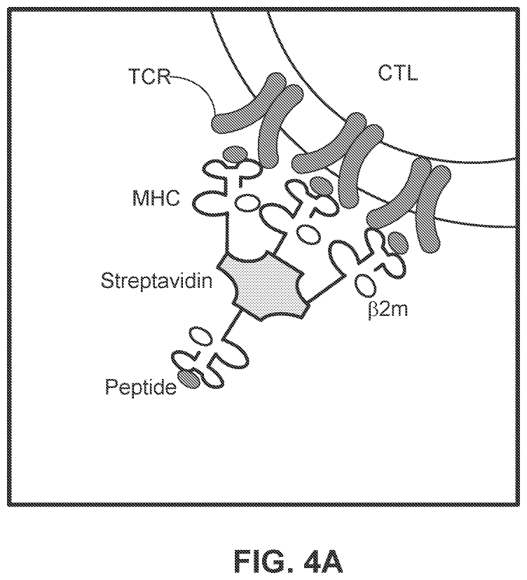

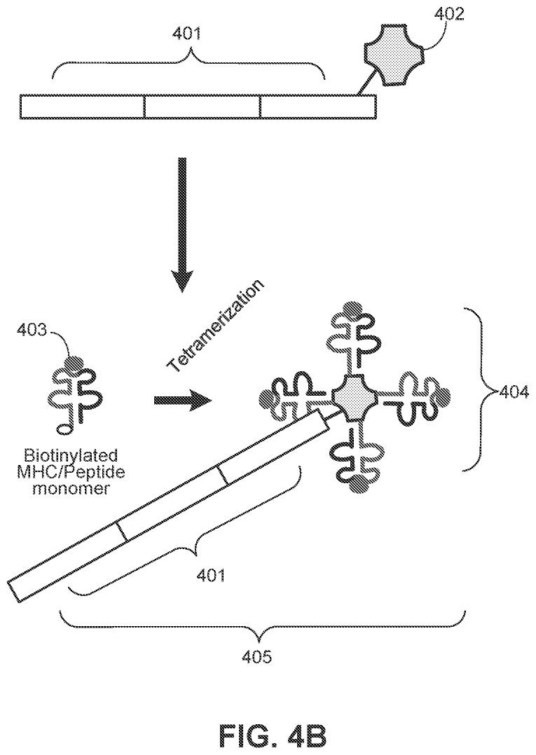

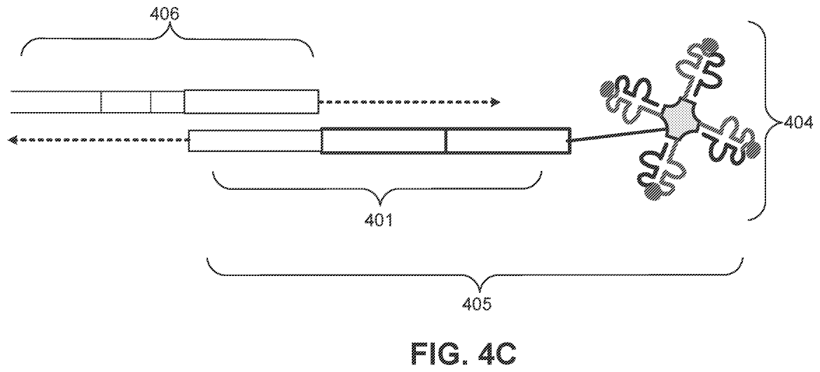

FIGS. 4A, 4B, and 4C are schematics illustrating how streptavidin cell tags can be utilized in an array-based system to produce a spatially-barcoded cell or cellular contents. For example, as shown in FIG. 4A, peptide-bound major histocompatibility complex (MHC) can be individually associated with biotin (β2m) and bound to a streptavidin moiety such that the streptavidin moiety comprises multiple pMHC moieties. Each of these moieties can bind to a TCR such that the streptavidin binds to a target T-cell via multiple MCH/TCR binding interactions. Multiple interactions synergize and can substantially improve binding affinity. Such improved affinity can improve labelling of T-cells and also reduce the likelihood that labels will dissociate from T-cell surfaces. As shown in FIG. 4B, a capture agent barcode domain 401 can be modified with streptavidin 402 and contacted with multiple molecules of biotinylated MHC 403 such that the biotinylated MHC 403 molecules are coupled with the streptavidin conjugated capture agent barcode domain 401. The result is a barcoded MHC multimer complex 405. As shown in FIG. 4B, the capture agent barcode domain sequence 401 can identify the MHC as its associated label and also includes optional functional sequences such as sequences for hybridization with other oligonucleotides. As shown in FIG. 4C, one example oligonucleotide is capture probe 406 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 406 may at first be associated with a feature (e.g., a gel bead) and released from the feature. In other embodiments, capture probe 406 can hybridize with a capture agent barcode domain 401 of the MHC-oligonucleotide complex 405. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of these corresponding sequences may be a complement of the original sequence in capture probe 406 or capture agent barcode domain 401. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 406 spatial barcode sequence may be used to identify a feature and the sequence derived from spatial barcode sequence on the capture agent barcode domain 401 may be used to identify the particular peptide MHC complex 404 bound on the surface of the cell (e.g., when using MHC-peptide libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product or an analyte capture agent, or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease (e.g., RNAse H). The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

As used herein, “immune cell infiltration” refers to presence, abundance and/or distribution of immune cells in one or more locations in a biological sample. For example, “immune cell infiltration” may refer to presence, abundance and/or distribution of tumor-infiltrating immune cells (e.g., tumor infiltrating lymphocytes (TILs) in one or more locations in a biological sample, such as a tumor tissue sample. The one or more locations in a biological sample can be a cancerous region (e.g., a tumor) in a biological sample. For example, immune cell infiltration may refer to presence, abundance and/or distribution of immune cells in a cancerous region in a biological sample, such as in a tumor. Additionally or in alternative, the one or more location in a biological sample can be a region surrounding a cancerous region (e.g., a stromal region) in a biological sample. For example, immune cell infiltration may refer to presence, abundance and/or distribution of immune cells in a region surrounding a cancerous region, such as in a stromal region. The one or more location in a biological sample can also be a cancer stromal region. For example, immune cell infiltration may refer to presence, abundance and/or distribution of immune cells in a cancer stromal region of a biological sample. In particular, methods and compositions of the present disclosure can be used for analyzing presence, abundance and/or distribution of infiltrating immune cells in one or more locations in a biological sample, such as in a cancer stromal region of a biological sample. For example, methods and compositions of the present disclosure can be used for analyzing presence, abundance and/or distribution of tumor infiltrating immune cells (e.g., TILs) in one or more locations in a biological sample, such as in a cancer stromal region of a biological sample.

As used herein, “immune cells” may refer to one or more cells associated with the immune system. In particular, the immune cells can be “infiltrating immune cells”, such as one or more immune cells infiltrating (i.e., present in) one or more locations in a biological sample, such as a cancerous region, a stromal region, and/or a cancer stromal region of a biological sample. Immune cells or infiltrating immune cells can include, without limitation, adaptive immune cells (e.g., a T cell or a B cell) and innate immune cells (e.g., Natural Killer (NK) cells, macrophages (e.g., tumor-associated macrophages (TAMs)), monocytes and dendritic cells (DCs). Non-limiting examples of infiltrating cells are as described, for example, in Zhang et al. (Cellul. Mol. Immuno., 17: 808-821 (2020)), which is herein incorporated by reference in its entirety. In some instances, the immune cell or infiltrating immune cell is an NK cell. NK cells are innate lymphoid cells that play a role in host immune response against tumor growth. NK cells can include the attributes as described in Melaiu et al., Front. Immunol., 10:1-18 (2020) and Zhang et al., Front. Immunol. 11: 1242 (2020), the entire contents of each are incorporated herein by reference. Presence of tumor-infiltrating NK cells has been linked with a good prognosis in multiple human solid tumors. In some embodiments, the NK cell is associated with an NKG7 analyte. Non-limiting examples of immune cell or infiltrating cells can include naïve B cells, memory B cells, plasma cells (a marker for a plasma cells includes, without limitation, CD79A, CD79B, CD38, CD27, MZB1, IGHA1, IGHG1, JCHAIN, and IGKC), CD8 T cells, CD4 naïve T cells, CD4 memory-resting T cells, CD4 memory-activated T cells, follicular helper T cells, regulatory T cells (Tregs) (a marker for a Treg includes, without limitation, FOXP3, IL17RB, CTLA4, FANK1, and CD4), gamma-delta T cells, resting NK cells, activated NK cells, monocytes, M0 macrophages, M1 macrophages, M2 macrophages, tissue associated macrophages (TAMs) (a marker for TAM includes, without limitation, CD163, MSR1, and MRC1), resting dendritic cells, activated dendritic cells, resting mast cells, activated mast cells, eosinophils, neutrophils, and any combinations thereof. In particular, an infiltrating immune cell can be a tumor infiltrating immune cell. A tumor infiltrating immune cell can be a tumor infiltrating lymphocyte (TIL), for example a T cell, and/or a B cell (TIB) (e.g., any of the exemplary B cells described herein, including plasma cells). Non-limiting examples of TILs are as described in Guo et al., (J. Oncol., doi: 10.1155/2019/2592419 (2019), the entire contents of which are incorporated herein by reference. In some instances, the TIL is selected from: (i) a CD3⁺ and CD4⁺ T cell; (ii) a CD3⁺ and CD8⁺ T cell; (iii) a regulatory T cell comprising one or more of: CD4, Foxp3, IL17RB, CTLA4, FANK1, HAVCR1, CD25, CTLA-4, GITR, LAG-3, and CD127; (iv) a TH1 cell comprising one or more of: CD4, CD3D, S100A4, IL7R, and IFNG; (v) a TH2 cell comprising one or more of: CD4, IL7R, ICOS, CTLA4, TNFRSF4, and TNFRS18; (vi) a TH17 cell comprising one or more of: CD4, CD3D, IL17A, GZMA, and S100A4; and (vii) a cytotoxic T cell comprising one or more of: CD8, CD3D, S100A4, IFNG, GZMB, GZMA, and IL2RB. In some instances, the tumor infiltrating B cell (TIB) is selected from: (i) a plasma cell comprising one or more of: MZB1, IGLL5, IGHA1, IGHG1, JCHAIN, IGKC, IGHA2, IGLC2, IGLV3-1, and IGLV2-14; (ii) an Ig⁺ B cells comprising one or more of: IGHV3-74, SOCS3, JCHAIN, and SPARC; (iii) an activated B cell comprising: CD79B, HMGB2, HMGB1, HMGN1, and RGS13; and (iv) a B cells comprising one or more of: MEF2B, RGS13, and MS4A1.

As used herein, a “cancerous region” of a biological sample may refer to one or more location of a biological sample that includes cancerous tissue. A cancerous region of a biological sample can be one or more locations in a tumor (e.g., pre-metastatic tumor, metastatic tumor, malignant tumor, etc.). In some instances where the biological sample has previously been identifying as including cancerous tissue, the cancerous region of the biological sample can represent a certain stage of the cancer. For example, a lung cancer sample can include cancerous region corresponding to different lung cancer stages, including tumor size T1, T2, T3, or T4. A cancerous region in a biological sample can be identified by one or more markers (e.g., biomarkers), such as Pan-CK. Other non-limiting examples of markers associated with a cancerous region include SCGB2A1, MKI67, BRCA1, BRCA2, PIKCD, CALML6, MYC, TP53, PALB2, RAD51, and/or MSH2.

As used herein, a “stromal region” of a biological sample may refer to one or more locations of a biological sample that is not a cancerous region. For example, a “stromal region” of a biological sample may refer to one or more locations that is outside the cancerous region of the biological sample. Additionally or in alternative, a stromal region of a biological sample can be a part of a tissue or organ with a structural or connective role. A stromal region of a biological sample can include one or more of connective tissue, blood vessels, and inflammatory cells. A stromal region in a biological sample can be identified by one or more markers (e.g., biomarkers), such as CD45.

### II. Detection of Immune Cell Infiltration Using Unbiased Approaches

This disclosure is based on using unbiased approaches to determine immune cell infiltration in a biological sample. In some instances, the spatial methods disclosed herein are combined with machine learning modules and gene clustering to identify areas of a sample that include tumor infiltrating immune cells.

This disclosure features methods of determining immune cell infiltration in a biological sample including one or more cancerous regions and one or more stromal regions in a subject where the method includes: (a) identifying a cancerous region or an analyte associated with the cancerous region from the one or more cancerous regions and/or identifying a stromal region or an analyte associated with the stromal region from the one or more stromal regions in the biological sample; (b) identifying one or more immune cells or an analyte associated with an immune cell in the cancerous region and/or the stromal region; and (c) determining the abundance of the one or more immune cells or the analyte associated with an immune cell in the biological sample; thereby determining immune cell infiltration in the biological sample. In some embodiments, the identifying the cancerous region, the identifying the stromal region, and/or the identifying immune cells includes: (a) generating a dataset from the biological sample, wherein the dataset includes one or more of (i) analyte data for a plurality of analytes captured from a plurality of spatial locations in the biological sample; (ii) image data comprising images of the plurality of spatial locations of the biological sample; and (iii) registration data linking the analyte data to the image data; and (b) using the dataset to identify the cancerous region, the stromal region, and/or the immune cells in the biological sample. In some embodiments, the identifying the cancerous region, the identifying the stromal region, and/or the identifying immune cells includes: (a) generating a dataset from the biological sample, wherein the dataset includes: (i) analyte data for a plurality of analytes captured from a plurality of spatial locations in the biological sample; (ii) image data comprising images of the plurality of spatial locations of the biological sample; and (iii) registration data linking the analyte data to the image data; and (b) using the dataset to identify the cancerous region, the stromal region, and/or the immune cells in the biological sample.

This disclosure features methods of determining immune cell infiltration in a biological sample comprising one or more cancerous regions and one or more stromal regions in a subject comprising: (a) generating a dataset from the biological sample obtained from the subject, wherein the dataset comprises: (i) analyte data for a plurality of analytes captured from a plurality of spatial locations of the biological sample, wherein an analyte in the plurality of analytes is an analyte associated with the cancerous region, an analyte associated with the stromal region, and/or an analyte associated with an immune cell; (ii) image data comprising images of the plurality of spatial locations of the biological sample; and (iii) registration data linking the analyte data to the image data; (b) providing the dataset to a trained machine learning module, wherein the trained machine learning module comprises reference analyte datasets from one or more reference samples, wherein the one or more reference samples comprises (i) a cancerous region from one or more cancerous regions, (2) a stromal region from one or more stromal regions, and (3) an immune cells from one or more immune cells; and (c) determining, via the trained machine learning module, the immune cell infiltration in the biological sample.

In some instances, the cancerous region comprises one or more of a benign tumor, a pre-metastatic tumor, a malignant tumor, and one or more inflammatory cells. In some instances, the stromal region comprises one or more of connective tissue, blood vessels, and inflammatory cells. Additional examples of cancerous and stromal regions will be apparent to one skilled in the art based on this disclosure.

(a) Determining Immune Cell Infiltration in a Biological Sample Using a Machine Learning Module

This disclosure features methods for determining immune cell infiltration in a biological sample using a machine learning module. In a non-limiting example, the disclosure features methods for determining immune cell infiltration in a biological sample comprising one or more cancerous regions and one or more stromal regions in a subject comprising: (a) generating a dataset from the biological sample obtained from the subject, wherein the dataset comprises: (i) analyte data for a plurality of analytes captured from a plurality of spatial locations of the biological sample, wherein an analyte in the plurality of analytes is an analyte associated with the cancerous region, an analyte associated with the stromal region, and/or an analyte associated with an immune cell; (ii) image data comprising images of the plurality of spatial locations of the biological sample; and (iii) registration data linking the analyte data to the image data; (b) providing the dataset to a trained machine learning module, wherein the trained machine learning module comprises reference analyte datasets from one or more reference samples, wherein the one or more reference samples comprises (i) a cancerous region from one or more cancerous regions, (2) a stromal region from one or more stromal regions, and (3) an immune cell from one or more immune cells; and (c) determining, via the trained machine learning module, the immune cell infiltration in the biological sample.

In some embodiments, a method for determining immune cell infiltration in a biological sample uses a machine learning module where the method includes: (a) generating a dataset of a plurality of biological samples, wherein the dataset includes, for each biological sample of the plurality of biological samples (e.g., including one or more reference sampled): (i) analyte data for a plurality of analytes captured from a plurality of spatial locations in the biological sample; (ii) image data comprising images of the plurality of spatial locations of the biological sample; and (iii) registration data linking the analyte data to the image data; wherein the reference biological sample includes (1) one or more cancerous regions in the reference biological sample, (2) one or more stromal regions within the one or more cancerous regions, and (3) a plurality of tumor infiltrating immune cells; (b) training a machine learning module with the dataset, thereby generating a trained machine learning module; and (c) using the trained machine learning module to determine immune cell infiltration in a test biological sample. In some embodiments, a dataset from a biological sample including (i) analyte data for a plurality of analytes captured from a plurality of spatial locations in the biological sample; (ii) image data comprising images of the plurality of spatial locations of the biological sample; and (iii) registration data linking the analyte data to the image data is provided to a trained machine learning module, wherein the trained machine learning module is trained at least in part from training data including one or more reference analyte datasets from one or more reference samples, wherein the one or more reference samples comprise (1) one or more reference cancerous regions, (2) one or more reference stromal regions, and (3) one or more reference immune cells.

In some embodiments, a method for determining immune cell infiltration in a biological sample includes: (a) accessing a dataset of a biological sample obtained from the subject, wherein the dataset includes (i) nucleic acid sequence data for a plurality of analytes captured from a plurality of spatial locations of the biological sample; (ii) image data comprising images of the plurality of spatial locations of the biological sample; and (iii) registration data linking the nucleic acid sequence data to the image data; (b) providing the dataset of the biological sample to a trained machine learning module; the trained machine learning module trained at least in part from training data comprising nucleic acid sequence datasets from one or more reference samples, the one or more reference samples comprising (1) one or more cancerous regions, (2) one or more stromal regions, and (3) one or more tumor infiltrating immune cells; (c) providing, via the trained machine learning module, an analysis of immune cell infiltration in cancer stroma of the subject.

In some embodiments, a computer implemented method can be used to train the machine learning module and determine, using the machine learning module, immune cell infiltration in a biological sample. In such cases, a computer implemented method includes: generating a dataset of a plurality of biological samples (e.g., one or more reference samples), wherein the dataset comprises, for each biological sample of the plurality of biological samples: (i) analyte data for a plurality of analytes captured at a plurality of spatial locations of a reference biological sample; (ii) image data of the reference biological sample; and (iii) registration data of the imaged data linking to the analyte data according to the spatial locations of the reference biological sample; wherein the reference biological sample comprises (1) one or more cancerous regions in the reference biological sample, (2) one or more stromal regions within the one or more cancerous regions, and (3) one or more immune cells; (b) training a machine learning module with the dataset, thereby generating a trained machine learning module; and (c) determining immune cell infiltration in a biological sample via the trained machine learning module.

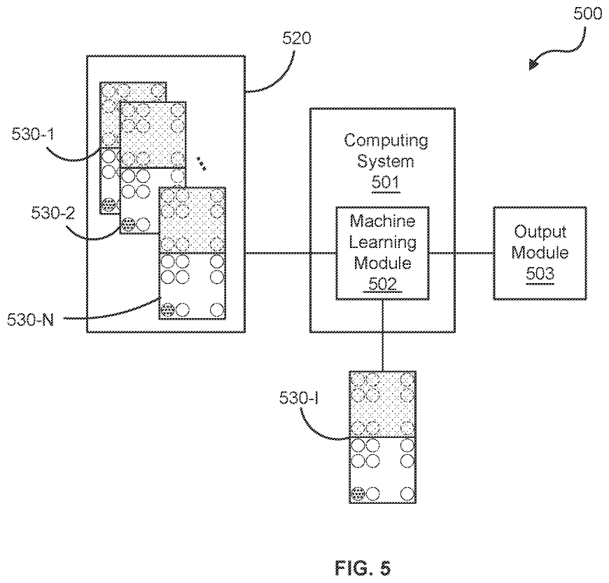

In some embodiments, an exemplary systems includes the components as described in the exemplary diagram as shown in FIG. 5. FIG. 5 shows a block diagram of an exemplary system 500 operable to identify a region of interest in a biological sample (e.g., a region of interest including a TIL). In this embodiment, the system 500 is implemented with a computing system 501. For example, the computing system 501 may include one or more processors, storage devices (e.g., persistent and volatile storage devices including computer memory, solid-state drives, hard disk drives, etc.), network interfaces, graphics cards, etc. The computing system 501 may be operable to implement a machine learning module 502. In this regard, the machine learning module 502 may be implemented as a combination of computer hardware, software, and/or firmware configured with the computing system 501.

In some embodiments, the computing system 501 may be operable to process a dataset of a plurality of data elements 530-1, 530-2 to 530-N (where the reference “N” is an integer greater than “1” and not necessarily equal to any other “N” reference designated herein). In some embodiments, each data element 530 includes data pertaining to captured and barcoded analytes of a biological sample. Each data element 530 may also include image data of the biological sample that is registered to the barcoded analytes. Imaging can be performed using any technique described herein.

For example, the biological sample may be interrogated with a plurality of capture probes at a plurality of capture areas, such as the capture spot (e.g., a spatially-barcoded feature) 101 of FIG. 1 as described herein. A capture area, as described herein, includes capture probes at particular locations on a substrate. Analytes (e.g., mRNA) released from the overlying cells of the biological sample can be captured by capture probes within the capture area on the substrate.

In some embodiments, the substrate including the capture probes also includes fiducial markers (e.g., any of the fiducial markers described herein or known in the art). For example, an image of the biological sample may be obtained with the fiducial markers. The fiducial markers of the image may be used to align the image of the biological sample with the data of the barcoded analytes at their known locations.

In some embodiments, the data elements 530 may each include a two-dimensional set of information pertaining to the biological sample. For example, the image may comprise a two-dimensional set of pixel data that includes pixel location, intensity, contrast, brightness, color (e.g., hue), etc. for each pixel in the image. This pixel data may be linked to the known locations of the capture areas (e.g., a spatially-barcoded feature) where the capture probes interrogate the biological sample. The data of the capture probes provides the third dimensional aspect of data of the data element 530.

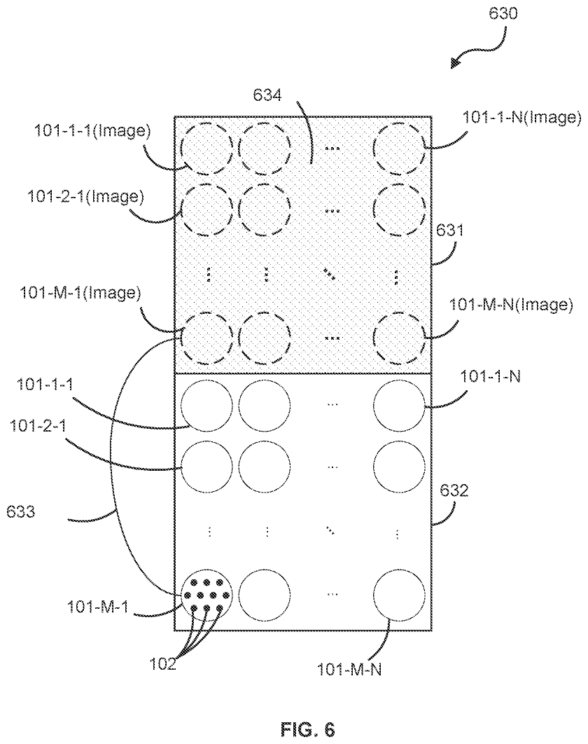

In some embodiments, an example data element is as shown and described in FIG. 6. In this embodiment, the data element 630 comprises an image 631 of a biological sample (not shown for simplicity) made up of a two-dimensional array of pixels 634. The image 631 in this embodiment is shown as an array of pixels for the purposes of illustration only as a display of the data pertaining to each of the pixels in the array would likely denigrate the understanding of the registration process.

In some embodiments, the data element 630 also comprises data from a substrate 632 (e.g., an M×N array) that includes capture areas (e.g., spatially-barcoded features) 101 where capture probes are used to interrogate the biological sample (wherein the references “M” and “N” are integers greater than “1” and not necessarily equal to any other “M” and “N” reference is designated herein). The data from these capture areas (e.g., spatially-barcoded features) 101 (e.g., the data of the barcoded analytes obtained therefrom) is linked to the image 631 to register the data of the barcoded analytes to the data of the pixels 634 of the image 631. For example, the capture area 101-M-1 of the biological sample comprises data from a plurality of barcoded analytes 102. This capture area (e.g., spatially-barcoded feature) 101-M-1 is linked (633) to a corresponding location 101-M-1 (Image) in the image 631 of the biological sample, thereby registering the data of the barcoded analytes to the pixel data of the image 631. In some embodiments, with the barcoded analytes 102 linked to the pixel locations of the image, various gene or proteins can be located such that gene or protein expressions (e.g., disease tissue, healthy tissue, the boundary of disease and healthy tissue, etc.) can be visualized or otherwise identified. In some embodiments, with the barcoded analytes 102 linked to the pixel locations of the image, various analytes can be located such that TIL-specific analytes or TIL-specific analyte signatures can be visualized or otherwise identified.

In some embodiments, obtaining data elements 630 from a plurality of samples may lend itself to machine learning (e.g., artificial intelligence processing). Machine learning generally regards algorithms and statistical models that computer systems, such as the computing system 501, use to perform a specific task without using explicit instructions, relying on patterns and inference instead. For example, machine learning algorithms may build a mathematical model based on sample data, known as “training data”, in order to make predictions or decisions without being explicitly programmed to perform the task. Thus, returning now to FIG. 5, when a plurality of biological samples is obtained from similar specimens (e.g., humans), a data element 630 from each biological sample may be generated to provide a dataset 520 that may be used to train the machine learning module 502 of the computing system 501.

In some embodiments, the machine learning module 502 may detect tumor infiltrating immune cells and/or identify various regions of interest in the biological samples that include tumor infiltrating immune cells. In one embodiment, the machine learning module 502 may operate on the dataset 520 to learn patterns in each of the data elements 530 to determine whether a similar pattern exists in a data element 530-I. For example, the dataset 520 may comprise data elements 530 obtained from biological samples of a diseased tissue of one specimen type. For example, the diseased tissue includes a cancerous region that includes TILs. The machine learning module 502 may be trained with each of the data elements 530 of the dataset 520 to learn patterns in image data and gene or protein expressions that may occur in such a diseased tissue. Thereafter, the machine learning module 502 may compare the learned patterns to any patterns in the data element 530-I such that an output module 503 may determine whether the biological sample yielding the data element 530-I has diseased tissue (e.g., has TILs present in the tissue specimen). In some embodiments, the machine learning module 502 may be operable to detect patterns within biological samples through the use of supervised learning. For example, an operator of the computing system 501 may identify patterns in an image of a sample that correspond to patterns in gene expressions. The operator may then use these identified patterns to train the machine learning module 502 such that the machine learning module 502 may detect similar patterns in subsequent data elements 530 input to the machine learning module 502. In another example, an operator of the computing system 501 may identify patterns in an image of a sample that correspond to patterns of one or more stains (e.g., any of the exemplary stains described herein). The operator may then use these identified patterns to train the machine learning module 502 such that the machine learning module 502 may detect similar patterns in subsequent data elements 530 input to the machine learning module 502.

In some embodiments, the training data may even be, or at least include, simulated data. For example, the physics and biology regarding biological processes of, e.g., disease tissue, healthy tissue, the boundary of disease and healthy tissue, etc. may be used as rules to generate data that can be formatted in a manner that would appear as the actual data (e.g., with barcode data registered to image data). This simulated data can be used either alone or in conjunction with the actual data to train the machine learning module 502.

In some embodiments, the machine learning module 502 includes one or more of a variety of machine learning algorithms. Non-limiting examples of machine learning algorithms that can be implemented by the machine learning module 502 include: a supervised learning algorithm, a semisupervised learning algorithm, an unsupervised learning algorithm, a regression analysis algorithm, a reinforcement learning algorithm, a self-learning algorithm, a feature learning algorithm, a sparse dictionary learning algorithm, an anomaly detection algorithm, a generative adversarial network algorithm, a transfer learning algorithm, and an association rules algorithm. In some embodiments, the machine learning module 502 is not intended to be limited to a particular machine learning algorithm. In some embodiments, non-limiting examples of machine learning algorithms that can be implemented by the machine learning module are as described in: Svensson et al., Nature Methods, 15: 343-346 (2018); Edsgard et al., Nature Methods, 15: 339-324 (2018); Sun et al., Nature Methods, 17(2): 193-200 (2020); J. N. R. Jeffers, J. Royal Stat. Society, Series D, 22(4) (1973), doi:10.2307/2986827; Hongfei et al., Geographical Analysis, 39(4): 357-275 (2007); Solomon Kullback, Information Theory and Statistics, ISBN 0-8446-5625-9 (Wiley 1978), the entire contents of each of which are incorporated herein by reference.

In some embodiments that include a transfer learning algorithm, knowledge gained while solving one problem could be applied to a different but related problem. For example, the machine learning module 502 can be trained using an initial type of data (e.g., image data, barcode data, etc.) to identify a relationship between a gene expression and an image pattern. The relationship between image data and the gene expression can be used in training the machine learning module 502 to identify a relationship between barcode data and the image data. In some embodiments, the machine learning module is not intended to be limited to any particular type or source of data, as data from a variety of sources and types may be used to train the machine learning module 502.

In some embodiments, the image data may be used to train the machine learning module 502 to identify locations in a sample that may include variations in the amount of a material in the sample. For example, a portion of an imaged sample may include a higher intensity, for example fluorescence, light or color intensity, than other portions of the image. This may indicate that there is more analyte (e.g., DNA, RNA, protein) at that location. This relationship may then be used to train the machine learning module 502 to identify DNA densities in other images. In another example, a portion of an imaged sample may include a higher intensity than other portions of the image, thereby indicating that there is more mRNA at that location. This relationship may then be used to train the machine learning module 502 to identify mRNA densities in other images. In yet another example, a portion of an imaged sample may include a higher intensity than other portions of the image, thereby indicating that there is more protein at that location. This relationship may then be used to train the machine learning module 502 to identify protein densities in other images.

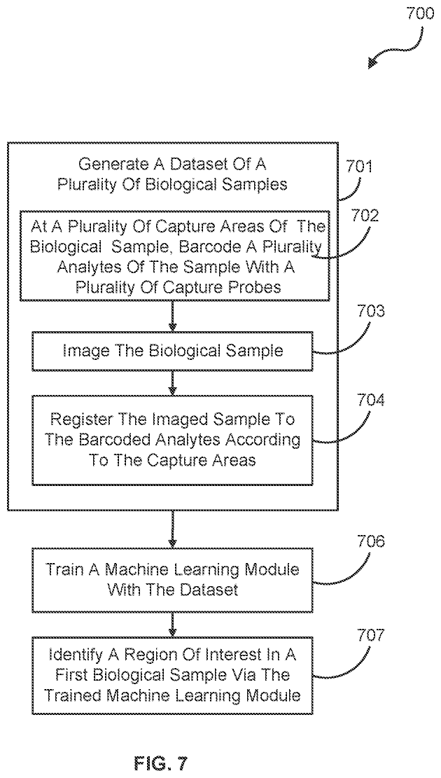

FIG. 5 and FIG. 7 show an exemplary process 700 of the computing system 500. In this embodiment, the process 700 initiates with the generation of a dataset 520 of a plurality of biological samples, in the process element 701. For example, a plurality of biological samples may be obtained from a particular specimen type, as described herein. In some embodiments, at a plurality of different locations in the biological sample (e.g., capture areas (e.g., spatially-barcoded features)), an analyte from the biological sample binds to a capture probe, the analyte is processed (e.g., capture probe extension and second strand synthesis) thereby creating a barcoded analyte (e.g., a sequence that includes a sequence of the analyte or a complement thereof, and a sequence of the barcode or a complement thereof) in the process element 702. The sample is imaged, in the process element 703, to produce a two-dimensional array of pixels from which the pixel data may be extracted. In some embodiments, the data pertaining to the barcoded analytes is registered to the image sample according to the capture areas (e.g., spatially-barcoded feature), in the process element 704.

In some embodiments, with the dataset 520 in hand, the computing system 501 trains the machine learning module 502 with the dataset 520, and in process element 706. Once trained, the machine learning module 502 may be operable to identify a region of interest in a first biological sample (e.g., the biological sample yielding the data element 530-I), in the process element 707. For example, the machine learning module 502 may be trained with data elements 530 pertaining to healthy tissue samples of a specimen so as to compare and contrast the data element 530-I with the data elements 530 of the dataset 520.

In some embodiments, a biological sample of the plurality of biological samples is a sample having previously been identified as having immune cell infiltration present in the biological sample. In some embodiments, a biological sample of the plurality of biological samples is a sample having not previously been identified as having immune cell infiltration present in the biological sample.

In some embodiments, a data set is generated for the biological sample. In some embodiments, the data set includes, without limitation, (i) analyte data for a plurality of analytes captured at a plurality of spatial locations (e.g., spatially-barcoded features) of the biological sample (e.g., where the biological sample is a test biological sample or one or more reference biological samples); (ii) image data comprising images of the plurality of spatial locations of the biological sample; and (iii) registration data linking the analyte data to the image data. In some embodiments, the data set is provided to a trained machine learning module, wherein the trained machine learning module is trained at least in part from training data comprising reference analyte datasets from one or more reference samples, wherein the one or more reference samples comprise (1) one or more reference cancerous regions, (2) one or more reference stromal regions, and (3) one or more reference immune cells. In some embodiments, the data set is used to train a machine learning module.

As used herein, “analyte data” can refer to data generated from detecting one or more analytes in the biological sample (e.g., a test biological sample or one or more reference biological samples), where detecting includes: attaching the one or more analytes from the test biological sample to a capture probe, wherein the capture probe includes a capture domain and a spatial barcode; and determining (i) all or a part of a sequence corresponding to the analyte, or a complement thereof, and (ii) all or a part of a sequence corresponding to the spatial barcode, or a complement thereof, and using the determined sequence of (i) and (ii) to identify the abundance and/or spatial location of the analyte in the test biological sample. In some embodiments, the analyte data may be used to train the machine learning module.

As used herein, “image data” can refer to data generated from obtaining an image of the biological sample; and registering the image data to a spatial location. In some embodiments, the image data includes obtaining images after the biological sample is stained with one or more stains. For example, the one or more stains can include hematoxylin and eosin. In some embodiments, the one or more stains comprise one or more optical labels. Non-limiting examples of optical labels includes: fluorescent, radioactive, chemiluminescent, calorimetric, or colorimetric labels.

In some embodiments, the image data can be used to identify one or more cancerous regions in the biological sample using the one or more stains of the biological sample. For example, image data can include obtaining an image of a biological sample stained with hematoxylin and eosin where the stain is used to identify one or more cancerous regions in the biological sample.

In some embodiments, the image data can be used to identify one or more stromal regions within the one or more cancerous regions using the one or more stains of the biological sample. For example, image data can include obtaining an image of a biological sample stained with hematoxylin and eosin where the stain is used to identify one or more stromal regions in one or more cancerous regions in the biological sample.

In some embodiments, the image data is registered to the analyte data. As used herein, “registration data” is data that links or compiles analyte data and image data in a data set as disclosed herein. For example, the imaged data is linked to the analyte data according to the spatial locations of the image data and the analyte data. In some embodiments, the image data may be used to train a machine learning module.

(b) Generating Analyte Data in a Biological Sample

This disclosure features methods for determining immune cell infiltration in a biological sample comprising one or more cancerous regions and one or more stromal regions in a subject, where the method includes generating analyte data. In some embodiments, the analyte data is from a cancerous region or an analyte associated with the cancerous region from the one or more cancerous regions; a stromal region or an analyte associated with the stromal region from the one or more stromal regions in the biological sample; and/or one or more immune cells or an analyte associated with an immune cell in the cancerous region and/or the stromal region. In some embodiments where the method includes generating analyte data, the method includes determining the abundance of one or more cancer regions or an analyte associated with the cancerous regions; one or more stromal regions or an analyte associated with the stromal region; and one or more immune cells or the analyte associated with an immune cell; thereby determining immune cell infiltration in the biological sample.

(i) Generating Analyte Data when the Analyte is a Nucleic Acid

This disclosure features methods for determining immune cell infiltration in a biological sample where the method includes capturing nucleic acids (e.g., mRNA and gDNA) on a substrate to identify immune cell infiltration. In some embodiments, the analyte associated with the In some embodiments, the method for determining immune cell infiltration in a biological sample includes generating a dataset of the biological sample including: contacting a biological sample from the subject having cancer with a substrate comprising a plurality of capture probes, wherein the biological sample comprises (1) one or more cancerous regions, (2) one or more stromal regions, and (3) one or more tumor infiltrating immune cells, and wherein a capture probe of the plurality of capture probes comprises a spatial barcode and a capture domain; attaching a nucleic acid molecule from the biological sample to the capture probe; determining (i) all or a part of a sequence corresponding to the nucleic acid molecule, or a complement thereof, and (ii) all or a part of a sequence corresponding to the spatial barcode, or a complement thereof, and using the determined sequence of (i) and (ii) to identify the spatial location and abundance of the nucleic acid molecule in the biological sample; and identifying a spatial location as being part of a cluster based on the determined sequences corresponding to the analytes at the spatial location and using the clusters to analyze immune cell infiltration in the cancer stroma of the subject having cancer.

In some embodiments, where the method for determining immune cell infiltration in a biological sample includes capture of nucleic acid molecules on a substrate, the method includes contacting the biological sample with a substrate including a plurality of capture probes, wherein a capture probe of the plurality of capture probes includes a spatial barcode and a capture domain; hybridizing the analyte associated with the cancerous region, the analyte associated with the stromal region, and/or the analyte associated with an immune cell to the capture probe; and determining (i) all or a part of a sequence corresponding to the analyte associated with the cancerous region, the analyte associated with the stromal region, and/or the analyte associated with an immune cell, or a complement thereof, and (ii) all or a part of a sequence corresponding to the spatial barcode, or a complement thereof, and using the determined sequence of (i) and (ii) to identify the abundance and/or spatial location of the analyte associated with the cancerous region, the analyte associated with the stromal region, and/or the analyte associated with an immune cell, or a complement thereof in the biological sample.

In some embodiments where the method for determining the location includes capture of nucleic acid molecules on a substrate, the determining step of the method includes sequencing (i) all or a part of a sequence corresponding to the nucleic acid molecule associated with the cancerous region, the nucleic acid molecule associated with the stromal region, and/or the nucleic acid molecule associated with an immune cell, or a complement thereof, and (ii) all or a part of a sequence corresponding to the spatial barcode, or a complement thereof, and using the determined sequence of (i) and (ii) to identify the abundance and/or spatial location of the nucleic acid molecule associated with the cancerous region, the nucleic acid molecule associated with the stromal region, and/or the nucleic acid molecule associated with an immune cell, or a complement thereof in the biological sample. In some embodiments, the sequencing includes in situ sequencing.

In some embodiments, the methods for determining immune cell infiltration in a biological sample where the method includes identifying a subset of nucleic acids based on the amount of analyte at the spatial location and the amount of the analyte at a plurality of different spatial locations in the biological sample; and sorting the subset of the analytes of (d) into a cluster based on the amount of the analytes at the plurality of different spatial locations in the biological sample, wherein one or more of the clusters includes analytes associated with a tumor infiltrating lymphocyte phenotype, and using the cluster(s) to identify the spatial location of the tumor infiltrating lymphocytes in the biological sample.

In some embodiments, the method for determining immune cell infiltration in a biological sample includes identifying analytes based on the amount of the analyte at the spatial location; and assigning the spatial location into a cluster based on the amount of the analyte at a given spatial location in the biological sample. In some embodiments, a cluster includes spatial locations wherein the analytes are associated with a tumor infiltrating immune cell phenotype. In some embodiments, a cluster includes spatial locations wherein the analytes are associated with a cancer cell phenotype. In some embodiments, a cluster includes spatial locations wherein the analytes are associated with a stromal cell phenotype. In some embodiments, spatial locations are grouped into a cluster based on the presence of one or more cancer analytes, one or more stromal region analytes, and/or immune cell analytes. In some embodiments, a cluster is used to identify immune cell infiltration in a biological sample.

Many methods can be used to help identify a cluster. Non-limiting examples of such methods include nonlinear dimensionality reduction methods such as t-distributed stochastic neighbor embedding (t-SNE), global t-distributed stochastic neighbor embedding (g-SNE), and uniform manifold approximation and projection (UMAP).

Any number of clusters can be identified. In some embodiments, 2 to 500 clusters can be identified using the methods as described herein. For example, 2 to 10, 2 to 20, 2 to 50, 2 to 75, to 100, 2 to 150, 2 to 200, 2 to 300, 2 to 400, 400 to 500, 300 to 500, 200 to 500, 100 to 500, 75 to 500, 50 to 500, or 25 to 200 clusters can be identified. In some embodiments, 25 to 75, 50 to 100, 50 to 150, 75 to 150, or 100 to 200 clusters can be identified. In some embodiments, 2 to 200 clusters are identified. In some embodiments, 2 to 10 clusters are identified.

In some embodiments, one or more analytes are detected using in situ sequencing. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the labeled primer identities (i.e., nucleotide sequence) the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363, the entire contents of each of which are incorporated herein by reference.

In addition, examples of methods and systems for performing in situ sequencing are described in PCT Patent Application Publication Nos. WO2014/163886, WO2018/045181, WO2018/045186, and in U.S. Pat. Nos. 10,138,509 and 10,179,932, the entire contents of each of which are incorporated herein by reference. Exemplary techniques for in situ sequencing include, but are not limited to, STARmap (described for example in Wang et al., (2018) Science, 361(6499) 5691), MERFISH (described for example in 2017/0220733 and in Moffitt, (2016) Methods in Enzymology, 572, 1-49), SeqFISH (described for example in U.S. Pat. No. 10,457,980), hybridization chain reaction amplification (described in U.S. Pat. No. 8,507,204) and FISSEQ (described for example in U.S. Patent Application Publication No. 2019/0032121). The entire contents of each of the foregoing references are incorporated herein by reference.

(ii) Generating Analyte Data when the Analyte is a Protein

This disclosure features methods for determining immune cell infiltration in a biological sample where the method includes using an analyte capture agent that includes an analyte binding moiety and an analyte binding moiety barcode to identify immune cell infiltration. In some embodiments, the method for determining immune cell infiltration in a biological sample includes generating a dataset of the biological sample including: attaching the biological sample with a plurality of analyte capture agents, wherein an analyte capture agent of the plurality of analyte capture agents includes: (i) an analyte binding moiety that binds specifically to the analyte associated with the cancerous region, the analyte associated with the stromal region, and/or the analyte associated with an immune cell; (ii) an analyte binding moiety barcode; and (iii) an analyte capture sequence, wherein the analyte capture sequence binds specifically to a capture domain; contacting the biological sample with a substrate, wherein the substrate includes a plurality of capture probes, wherein a capture probe of the plurality of capture probes includes (i) the capture domain and (ii) a spatial barcode; hybridizing the analyte associated with the cancerous region, the analyte associated with the stromal region, and/or the analyte associated with an immune cell to the capture probe; and determining (i) all or a part of a sequence corresponding to the analyte associated with the cancerous region, the analyte associated with the stromal region, and/or the analyte associated with an immune cell, or a complement thereof, and (ii) all or a part of a sequence corresponding to the spatial barcode, or a complement thereof, and using the determined sequence of (i) and (ii) to identify the abundance and/or spatial location of the analyte associated with the cancerous region, the analyte associated with the stromal region, and/or the analyte associated with an immune cell, or a complement thereof in the biological sample.

In some embodiments, where the method for determining immune cell infiltration in a biological sample includes using an analyte capture agent, the method includes: attaching the biological sample with a plurality of analyte capture agents, wherein an analyte capture agent of the plurality of analyte capture agents includes: (i) an analyte binding moiety that binds specifically to the analyte associated with the cancerous region, the analyte associated with the stromal region, and/or the analyte associated with an immune cell; (ii) an analyte binding moiety barcode; and (iii) an analyte capture sequence, wherein the analyte capture sequence binds specifically to a capture domain; contacting the biological sample with a substrate, wherein the substrate includes a plurality of capture probes, wherein a capture probe of the plurality of capture probes includes (i) the capture domain and (ii) a spatial barcode; hybridizing the analyte associated with the cancerous region, the analyte associated with the stromal region, and/or the analyte associated with an immune cell to the capture probe; and determining (i) all or a part of a sequence corresponding to the analyte associated with the cancerous region, the analyte associated with the stromal region, and/or the analyte associated with an immune cell, or a complement thereof, and (ii) all or a part of a sequence corresponding to the spatial barcode, or a complement thereof, and using the determined sequence of (i) and (ii) to identify the abundance and/or spatial location of the analyte associated with the cancerous region, the analyte associated with the stromal region, and/or the analyte associated with an immune cell, or a complement thereof in the biological sample.

In some embodiments where the method for determining the location includes using an analyte capture agent, the determining step of the method includes sequencing (i) all or a part of a sequence corresponding to the analyte associated with the cancerous region, the analyte associated with the stromal region, and/or the analyte associated with an immune cell, or a complement thereof, and (ii) all or a part of a sequence corresponding to the spatial barcode, or a complement thereof, and using the determined sequence of (i) and (ii) to identify the abundance and/or spatial location of the analyte associated with the cancerous region, the analyte associated with the stromal region, and/or the analyte associated with an immune cell, or a complement thereof in the biological sample. In some embodiments, the sequencing includes in situ sequencing.

An “analyte capture agent” refers to a molecule that interacts with a target analyte and with a capture probe to identify the analyte. In some embodiments, an analyte capture agent includes a label (e.g., fluorescent label). In some embodiments, the analyte capture agent can include an analyte binding moiety and a capture agent barcode domain. An analyte binding moiety is a molecule capable of binding to a specific analyte. In some embodiments, the analyte binding moiety includes an antibody or antibody fragment. In some embodiments, the analyte binding moiety includes a polypeptide and/or an aptamer. In some embodiments, the analyte binding moiety includes a DNA aptamer. In some embodiments, the analyte binding moiety includes a RNA aptamer. In some embodiments, the analyte binding moiety includes an aptamer of mixed natural or unnatural occurring nucleotides (e.g., LNA, PNA). In some embodiments, the analyte is a protein (e.g., a protein on a surface of a cell or an intracellular protein). In some embodiments, the analyte binding moiety is an antibody or antigen-binding fragment thereof, a cell surface receptor binding molecule, a receptor ligand, a small molecule, a T-cell receptor engager, a B-cell receptor engager, a pro-body, an aptamer, a monobody, an affimer, or a darpin. In some embodiments, the method includes: contacting the biological sample with a fluorescently-labeled antibody.

A capture agent barcode domain can include an analyte capture sequence which can hybridize to at least a portion or an entirety of a capture domain of a capture probe. In some embodiments, the analyte capture sequence includes a poly (A) tail. In some embodiments, the analyte capture sequence includes a sequence capable of binding a poly (T) domain. In some embodiments, the analyte capture sequence can have a GC content between 1-100%, e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 70%, 80%, etc.). In some embodiments, the analyte capture sequence has a GC content of at least 30%. In some embodiments, one or more pluralities of analyte capture agents can be provided to a biological sample, wherein one plurality of analyte capture agent differs from another plurality of analyte capture agent by the analyte capture sequence. For example, analyte capture sequence A can be correlated with analyte binding moiety A, and analyte capture sequence B can be correlated with analyte binding moiety B. In some embodiments, the two pluralities of analyte capture agents can have the same analyte binding moiety barcode sequence.

In some embodiments, the capture domain includes a poly (T) tail. In some embodiments, the capture domain includes a sequence capable of binding a poly (A) domain. In some embodiments, the capture domain can have a GC content between 1%-100%, e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 70%, 80%, etc. In some embodiments, the capture domain has a GC content of at least 30%.

In some embodiments, the capture agent barcode domain includes an analyte binding moiety barcode. The analyte binding moiety barcode refers to a barcode that is associated with or otherwise identifies the analyte binding moiety. In some embodiments, the analyte binding moiety barcode is correlated with the type of analyte binding moiety, such that more than one plurality of analyte capture agents can be provided to a biological sample at one time. For example, analyte binding moiety barcode A can be correlated with analyte binding moiety A, and analyte binding moiety barcode B is correlated with analyte binding moiety B. The two pluralities of analyte capture agents can have the same analyte capture sequence (e.g., poly(A)). In some embodiments, one analyte binding moiety barcode plurality is correlated with one analyte capture sequence plurality. In other embodiments, an analyte binding moiety barcode plurality is not necessarily correlated with an analyte capture sequence plurality.

In some embodiments, a capture agent barcode domain includes optional sequences, such as, without limitation, a PCR handle, a sequencing priming site, a domain for hybridizing to another nucleic acid molecule, and combinations thereof. In some embodiments, the PCR handle is identical on all capture analyte barcode domains. In some embodiments, the PCR handle is included for PCR amplification. In some embodiments, an analyte capture agent includes one or more optional sequences and one or more barcode sequences (e.g., one or more analyte binding moiety barcodes and/or one or more UMIs). In some embodiments, the capture probe capture domain and/or the analyte capture agent include a cleavage domain. In some embodiments, a capture agent barcode domain can be dissociated from the analyte binding moiety by cleaving the analyte binding moiety from the capture agent barcode domain via a cleavage domain in the capture agent barcode domain. Other embodiments of an analyte capture agent useful in spatial protein detection are described herein.

Provided herein are methods for spatially profiling a biological analyte, e.g., any of the analytes as described herein, in a biological sample that use a spatially-tagged analyte capture agent. A biological analyte can be bound by an analyte capture agent at a distinct spatial position on a substrate and detected. The bound biological analyte can then be correlated with a barcode of the capture probe at a distinct spatial position of the substrate. In some embodiments, these methods can include spatially profiling the biological analyte from one or more of: an intracellular region of a cell in a biological sample, a cell surface region of a cell in a biological sample, a particular type of cell in a biological sample, and a region of interest of a biological sample.

(a) Blocking Probes

In some embodiments, an analyte capture sequence of a capture agent barcode domain is blocked prior to adding the analyte capture agent to a biological sample. In some embodiments, an analyte capture sequence of a capture agent barcode domain is blocked prior to adding the analyte capture agent to a capture probe array. In some embodiments, blocking probes are added to blocking buffer or other solutions applied in an IHC and/or IF protocol. In some embodiments, a blocking probe is used to block or modify the free 3′ end of the capture agent barcode domain. In some embodiments, a blocking probe is used to block or modify the free 3′ end of the analyte capture sequence of the capture agent barcode domain. In some embodiments, a blocking probe can be hybridized to the analyte capture sequence of a capture agent barcode domain to mask the free 3′ end of the capture agent barcode domain. In some embodiments, a blocking probe can be a hairpin probe or partially double stranded probe. In some embodiments, the free 3′ end of the analyte capture sequence of the capture agent barcode domain can be blocked by chemical modification, e.g., addition of an azidomethyl group as a chemically reversible capping moiety such that the capture probes do not include a free 3′ end. Blocking or modifying the capture agent barcode domains, particularly at the free 3′ end of the capture agent barcode domain, prior to contacting the analyte capture agents with the substrate, prevents binding of the analyte capture sequence to capture probe capture domain (e.g., prevents the binding of an analyte capture sequence poly(A) tail to a poly(T) capture domain).

In some embodiments, a blocking probe is used to block or modify the free 3′ end of a capture probe. In some embodiments, a blocking probe is used to block or modify the free 3′ end of a capture probe capture domain. In some embodiments, the analyte capture sequence is blocked prior to adding the analyte capture agent to a capture probe array. In some embodiments, blocking probes are added to blocking buffer or other solutions applied in an IHC and/or IF protocol. In some embodiments, a blocking probe can be hybridized to the capture domain to mask the free 3′ end of the capture domain. In some embodiments, a blocking probe can be a hairpin probe or partially double stranded probe. In some embodiments, the free 3′ end of the capture domain can be blocked by chemical modification, e.g., addition of an azidomethyl group as a chemically reversible capping moiety such that the capture probes do not include a free 3′ end. Blocking or modifying the capture domains, particularly at the free 3′ end of the capture domain, prior to contacting the analyte capture agents with the capture probe array, prevents binding of the analyte capture sequence to capture probe capture domain (e.g., prevents the binding of an analyte capture sequence poly(A) tail to a poly(T) capture domain).

In some embodiments, the blocking probes can be reversibly removed. For example, blocking probes can be applied to block the free 3′ end of either or both the capture agent barcode domain and/or the capture probes. Blocking interaction between the analyte capture agent and the capture probe array can reduce non-specific background staining in IHC and/or IF applications. After the analyte binding agents are bound to the target analyte, the blocking probes can be removed from the 3′ end of the capture agent barcode domain and/or the capture probe, and the analyte-bound analyte binding agents can migrate to and become bound by the capture probe array. In some embodiments, the removal includes denaturing the blocking probe from the analyte binding moiety barcode and/or capture probe. In some embodiments, the removal includes removing a chemically reversible capping moiety. In some embodiments, the removal includes digesting the blocking probe with an RNAse (e.g., RNAse H).

In some embodiments, the blocking probes are oligo (dT) blocking probes. In some embodiments, the oligo (dT) blocking probes can have a length of 15-30 nucleotides. In some embodiments, the oligo (dT) blocking probes can have a length of 10-50 nucleotides, e.g., 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50 nucleotides. In some embodiments, the analyte capture agents can be blocked at different temperatures (e.g., 4° C. and 37° C.). In some embodiments, the analyte capture agents can be blocked from binding to the capture probes more effectively at lower temperatures when using shorter blocking probes.

(b) Spatially-Tagged Capture Agents

A “spatially-tagged analyte capture agent” can be a molecule that interacts with an analyte (e.g., an analyte in a sample) and with a capture probe to identify the spatial location of the analyte. In some embodiments, a spatially-tagged analyte capture agent can be an analyte capture agent with an extended capture agent barcode domain that includes a sequence complementary to a spatial barcode of a capture probe. In some embodiments, an analyte capture agent is introduced to an analyte and a capture probe at the same time. In some embodiments, an analyte capture agent is introduced to an analyte and a capture probe at different times. In some embodiments, the spatially-tagged analyte capture agent is denatured from the capture probe before the biological sample is introduced. In some embodiments, the spatially-tagged analyte capture agent binds to a biological analyte within a biological sample before the spatially-tagged analyte capture agent is denatured from the capture probe. In some embodiments, the capture probe is cleaved from the substrate while attached to the spatially-tagged analyte capture agent. In some embodiments, once the capture domain of the capture probe is bound to the analyte binding moiety barcode, the analyte capture sequence is extended towards the 3′ tail to include a sequence that is complementary to the sequence of the capture probe spatial barcode (e.g., producing a spatially-tagged analyte capture agent).

For example, an analyte capture agent can be introduced to a biological sample, wherein the analyte binding moiety binds to a target analyte, and then the biological sample can be treated to release the analyte-bound analyte capture agent from the sample. The analyte-bound analyte capture agent can then migrate and bind to a capture probe capture domain, and the analyte-bound capture agent barcode domain can be extended to generate a spatial barcode complement at the end of the capture agent barcode domain. The analyte-bound spatially-tagged analyte capture agent can be denatured from the capture probe, and analyzed using methods described herein.

In another example, an analyte capture agent can be hybridized to a capture probe capture domain on a capture probe array, wherein the capture agent barcode domain is extended to include a sequence complementary to the spatial barcode of the capture probe. A biological sample can be contacted with the analyte capture agent modified capture probe array. Analytes from the biological sample can be released from the sample, migrated to the analyte capture agent modified capture probe array, and captured by an analyte binding moiety. The capture agent barcode domain of the analyte-bound analyte capture agents can be denatured from the capture probe, and the biological sample can be dissociated and spatially processed according to methods described herein.

In some embodiments, a spatially-tagged analyte capture agent can attach to a surface of a cell through a combination of lipophilic and covalent attachment. For example, a spatially-tagged analyte capture agent can include an oligonucleotide attached to a lipid to target the oligonucleotide to a cell membrane, and an amine group that can be covalently linked to a cell surface protein(s) via any number of chemistries described herein. In these embodiments, the lipid can increase the surface concentration of the oligonucleotide and can promote the covalent reaction.

(c) Generating Image Data in a Biological Sample

This disclosure features methods for determining immune cell infiltration in a biological sample comprising one or more cancerous regions and one or more stromal regions in a subject, where the method includes generating image data. In some embodiments, the image data is from a cancerous region or an analyte associated with the cancerous region from the one or more cancerous regions; a stromal region or an analyte associated with the stromal region from the one or more stromal regions in the biological sample; and/or one or more immune cells or an analyte associated with an immune cell in the cancerous region and/or the stromal region. In some embodiments where the method includes generating image data, the method includes determining the abundance of one or more cancer regions or an analyte associated with the cancerous regions; one or more stromal regions or an analyte associated with the stromal region; and one or more immune cells or the analyte associated with an immune cell; thereby determining immune cell infiltration in the biological sample.

In some embodiments, the image data is generated using a method comprising obtaining an image of the biological sample; and registering the image data to a spatial location. In some embodiments, the method includes identifying (1) the one or more cancerous regions; and/or (2) the one or more stromal regions based on the image data. In some embodiments, the method also includes identifying the one or more immune cells based on the image data. In some embodiments, obtaining an image of the biological sample; and registering the image data to a spatial location. In some embodiments, further comprising identifying the one or more cancerous regions and the one or more stromal regions via the trained machine learning module. In some embodiments, the method also includes identifying the one or more immune cells via the trained machine learning module.

In some embodiments, the determining the abundance of immune cells in the biological sample includes: identifying the one or more cancer regions including: obtaining an image and registering the image data to the spatial location, using the spatial location of the determined sequences, or obtaining an image and registering the image data to the spatial location, and using the spatial location of the determined sequences; identifying the one or more stromal regions including: obtaining an image and registering the image data to the spatial location, using the spatial location of the determined sequences, or obtaining an image and registering the image data to the spatial location, and using the spatial location of the determined sequences; and identifying the abundance of one or more immune cell infiltrates including: obtaining an image and registering the image data to the spatial location, using the spatial location of the determined sequences, or obtaining an image and registering the image data to the spatial location, and using the spatial location of the determined sequences.

In some embodiments, the method of determining immune cell infiltration includes determining the abundance of immune cells in the biological sample. In some embodiments, the abundance of immune cells in the biological sample includes about 5%, about 10%, about 15%, about 20%, about 25%, about 30%, about 35%, about 40%, about 45%, or about 50% of the cells in the biological sample. In some embodiments, the abundance of immune cells in the biological sample includes is about 5%, about 10%, about 15%, about 20%, about 25%, about 30%, about 35%, about 40%, about 45%, or about 50% of the cells in the cancer region. In some embodiments, the abundance of immune cells in the biological sample includes is about 5%, about 10%, about 15%, about 20%, about 25%, about 30%, about 35%, about 40%, about 45%, or about 50% of the cells in the stromal region.

In some instances, biomarkers of the cancerous and/or the stromal region could be used to determine the cancerous and/or stromal regions. In some instances, immunohistochemistry or immunofluorescence can be used to detect these regions of interest. In some instances, Pan-CK can be used to detect cancerous regions. In some instances, CD45 can be used to detect stromal regions. Any method of biomarker (e.g., protein) detection can be used to determine the regions of interest, including but not limited to, immunofluorescence (i.e., using primary and optionally secondary antibodies to visualize the biomarker). In some instances, provided herein are methods of detecting overlap of expression of Pan-CK or CD45 with cancerous markers or stromal biomarkers, respectively. In some instances, the cancerous markers that overlap with Pan-CK expression include PRKCI, VTCN1, MECOM, TOP2A, SHDH, XPO1, TFRC, FUT8, SOX17, PBX1, EIF42, and WTT. Non-limiting examples of analytes associated with an immune infiltrating cell can also include byproducts, precursors, and degradation products of such analytes thereof, and any combination of such analytes and byproducts, precursors, and degradation products thereof. In some instances, the cancerous markers that overlap with Pan-CK expression include VTCN1, MECOM, TOP2A, XPO1, FUT8, SOX17, PBX1, EIF42, and WTT. Non-limiting examples of analytes associated with an immune infiltrating cell can also include byproducts, precursors, and degradation products of such analytes thereof, and any combination of such analytes and byproducts, precursors, and degradation products thereof.

In some embodiments, the determining comprises identifying the amount of genes associated with immune infiltrating cells compared to known housekeepers normalized by number of cells per spatial location. In some embodiments, the determining comprises identifying the ratio of one or more tumor infiltrating lymphocytes (TILs) to one or more tumor infiltrating B cells (TIBs). In some embodiments, the determining comprises calculating the abundance of tumor infiltrating immune cells in the biological sample based on the percentage of spatial locations comprising analytes associated with an immune infiltrating cells.

In some embodiments, the identification of the one or more cancerous regions includes segmenting the cancerous regions from the image data. In some embodiments, the identification of the one or more stromal regions includes segmenting the stromal regions from the image data. In some embodiments, the identification of the one or more immune cells includes segmenting immune cells from the image data. In some embodiments, the abundance of immune cells in the cancer stromal region is determined using segmenting and (i) obtaining an image and registering the image data to the spatial location, (ii) using the spatial location of the determined sequences, or (iii) obtaining an image and registering the image data to the spatial location, and using the spatial location of the determined sequences.

As used herein, the term “segmenting” can refer to the process of partitioning a biological sample into multiple segments (e.g., without limitation, portions, partitions, regions of interest, and single cells). “Segmenting” and segmentation” can be used interchangeably. In some embodiments, segmenting includes determining the boundaries of one or more biological segments (e.g., one or more cancerous regions, one or more stromal regions, and one or more immune cells). In some cases, segmentation can be done manually (e.g., visual inspection by a pathologist), with gene or protein expression data, and/or using a trained machine learning module.

(d) Slides, Biological Samples, and Analytes

This disclosure features a method for determining immune cell infiltration in a biological sample using a substrate (e.g., a first substrate) that includes a plurality of capture probes, where a capture probe of the plurality of capture probes include a capture domain but no spatial barcode. In some embodiments, the capture probe is affixed to the substrate at a 5′ end. In some embodiments, the plurality of capture probes are uniformly distributed on a surface of the substrate. In some embodiments, the plurality of capture probes are located on a surface of the substrate but are not distributed on the substrate according to a pattern. In some embodiments, the substrate (e.g., a second substrate) includes a plurality of capture probes, where a capture probe of the plurality of capture probes includes a capture domain and a spatial barcode.

In some embodiments, the capture domain includes a sequence that is at least partially complementary to the analyte or the analyte derived molecule. In some embodiments, the capture domain of the capture probe includes a poly(T) sequence. In some embodiments, the capture domain includes a functional domain. In some embodiments, the functional domain includes a primer sequence. In some embodiments, the capture probe includes a cleavage domain. In some embodiments, the cleavage domain includes a cleavable linker from the group consisting of a photocleavable linker, a UV-cleavable linker, an enzyme-cleavable linker, or a pH-sensitive cleavable linker.

In some embodiments, the biological sample includes a FFPE sample. In some embodiments, the biological sample includes a tissue section. In some embodiments, the biological sample includes a fresh frozen sample. In some embodiments, the biological sample includes live cells.

In some embodiments, the biological sample comprises brain tissue, a spinal cord tissue, a skin tissue, an adipose tissue, an intestinal tissue, a colon tissue, a cervical tissue, a vaginal tissue, a muscle tissue, a cardiac tissue, a liver tissue, a pancreatic tissue, a kidney tissue, a spleen tissue, a lymph node tissue, a bone marrow tissue, a cartilage tissue, a retinal tissue, a corneal tissue, a breast tissue, a prostate tissue, a bladder tissue, a tracheal tissue, a lung tissue, a uterine tissue, a stomach tissue, a thyroid tissue, a thymus tissue, or a combination thereof. In some embodiments, the biological sample is obtained from a biopsy. Non-limiting examples of biopsy samples include: core needle biopsies and fine needle aspiration. In some embodiments, the biological sample is obtained from a surgical excision. In some embodiments, the biological sample was collected during an endoscopy or colposcopy. In some embodiments, the biological sample is collected during an endoscopy or colonoscopy. In some embodiments, the biological sample or comprises cerebrospinal fluid, whole blood, plasma, and/or serum.

In some embodiments, the biological sample (e.g., a reference biological sample, or a test biological sample) is a sample that has previously been identified as including cancerous tissue. In some embodiments where the biological sample has previously been identifying as including cancerous tissue, the biological sample represents a certain stage of the cancer (e.g., lung cancer stages including tumor size T1, T2, T3, or T4).

The methods provided herein can be applied to analyte or analyte derived molecules including, without limitation, a second strand cDNA molecule (“second strand”). In some embodiments, the analyte or analyte derived molecules include RNA and/or DNA. In some embodiments, the analyte is a protein.

This disclosure features methods for determining immune cell infiltration in a biological sample where the methods include determining the abundance and/or spatial location of analyte associated with an immune infiltrating cell. Non-limiting examples of analytes associated with an immune infiltrating cell include: BLK, CD19, FCRL2, MS4A1, KIAA0125, TNFRSF17, TCL1A, SPIB, PNOC, PTRPC, PRF1, GZMA, GZMB, NKG7, GZMH, KLRK1, KLRB1, KLRD1, CTSW, GNLY, CCL13, CD209, HSD11B1, LAG3, CD244, EOMES, PTGER4, CD68, CD84, CD163, MS4A4A, TPSB2, TPSAB1, CPA3, MS4A2, HDC, FPR1, SIGLEC5, CSF3R, FCAR, FCGR3B, CEACAM3, S100A12, KIR2DL3, KIR3DL1, KIR3DL2, IL21R, XCL1, XCL2, NCR1, CD6, CD3D, CD3E, SH2D1A, TRAT1, CD3G, TBX21, FOXP3, CD8A, CD8B, CD79A, CD79B, CD4, IGHA1, IGHG2, JCHAIN, IGKC, CD27, CD38, CD16, IL17RB, FANK1, CTLA4, MSR1, MRC1, NKG7, FCN1, and TIGIT/LAG3. Non-limiting examples of analytes associated with an immune infiltrating cell can also include byproducts, precursors, and degradation products of such analytes thereof, and any combination of such analytes and byproducts, precursors, and degradation products thereof. In some embodiments, the methods of determining immune cell infiltration in the biological sample includes identifying abundance and/or spatial location of an analyte associated with an immune infiltrating cell in a biological sample includes determining the abundance and/or spatial location of a housekeeping analyte. Non-limiting examples of housekeeping analytes that can be used in the methods described herein are as described in Eisenberg et al., Trends in Genetics, 29(10): 569-574 (2013) and Waxman et al., BMC Genomics, 8:243 (2007), the entire contents of each are incorporated herein by reference. In some embodiments, a housekeeping analyte can include, without limitations, glyceraldehyde-3-phosphate dehydrogenase (GAPDH), TATA-binding protein (TBP), and ribosomal proteins (RP). In some embodiments, the method includes identifying the ratio of one or more analyte associated with an immune infiltrating cell to a housekeeping analyte in the biological sample (e.g., in one or more cancerous regions).

This disclosure features methods for determining immune cell infiltration in the cancer stroma of a patient having cancer where the immune cell is a tumor infiltrating lymphocyte (TIL), for example a T cell, and/or a B cell (TIB) (e.g., any of the exemplary B cells described herein, including plasma cells). Non-limiting examples of TILs are as described in Guo et al., (J. Oncol., doi: 10.1155/2019/2592419 (2019), the entire contents of which are incorporated herein by reference.

In some embodiments, the TIL is selected from: (i) a CD3⁺ and CD4⁺ T cell; (ii) a CD3⁺ and CD8⁺ T cell; (iii) a regulatory T cell comprising one or more of: CD4, Foxp3, IL17RB, CTLA4, FANK1, HAVCR1, CD25, CTLA-4, GITR, LAG-3, and CD127; (iv) a TH1 cell comprising one or more of: CD4, CD3D, S100A4, IL7R, and IFNG; (v) a TH2 cell comprising one or more of: CD4, IL7R, ICOS, CTLA4, TNFRSF4, and TNFRS18; (vi) a TH17 cell comprising one or more of: CD4, CD3D, IL17A, GZMA, and S100A4; and (vii) a cytotoxic T cell comprising one or more of: CD8, CD3D, S100A4, IFNG, GZMB, GZMA, and IL2RB.

In some embodiments, the tumor infiltrating B cell (TIB) is selected from: (i) a plasma cell comprising one or more of: MZB1, IGLL5, IGHA1, IGHG1, JCHAIN, IGKC, IGHA2, IGLC2, IGLV3-1, and IGLV2-14; (ii) an Ig⁺ B cells comprising one or more of: IGHV3-74, SOCS3, JCHAIN, and SPARC; (iii) an activated B cell comprising: CD79B, HMGB2, HMGB1, HMGN1, and RGS13; and (iv) a B cells comprising one or more of: MEF2B, RGS13, and MS4A1.

This disclosure features methods of identifying abundance and/or spatial location of an infiltrating immune cell, where an infiltrating immune cell includes, without limitation, adaptive immune cells (e.g., a T cell or a B cell) and innate immune cells (e.g., Natural Killer (NK) cells, macrophages (e.g., tumor-associated macrophages (TAMs)), monocytes and dendritic cells (DCs). Non-limiting examples of infiltrating cells are as described in Zhang et al. (Cellul. Mol. Immuno., 17: 808-821 (2020)), which is herein incorporated by reference in its entirety.

In some embodiments, the immune infiltrating cell is an NK cell. NK cells are innate lymphoid cells that play a role in host immune response against tumor growth. NK cells can include the attributes as described in Melaiu et al., Front. Immunol., 10:1-18 (2020) and Zhang et al., Front. Immunol. 11: 1242 (2020), the entire contents of each are incorporated herein by reference. Presence of tumor-infiltrating NK cells has been linked with a good prognosis in multiple human solid tumors. In some embodiments, the NK cell is associated with an NKG7 analyte.

In some embodiments, the infiltrating immune cells identified using the methods disclosed herein include, but are not limited to, naïve B cells, memory B cells, plasma cells (e.g., a marker for a plasma cells includes, without limitation, CD79A, CD79B, CD38, CD27, MZB1, IGHA1, IGHG1, JCHAIN, and IGKC) CD8 T cells, CD4 naïve T cells, CD4 memory-resting T cells, CD4 memory-activated T cells, follicular helper T cells, regulatory T cells (Tregs) (e.g., a marker for a Treg includes, without limitation, FOXP3, IL17RB, CTLA4, FANK1, and CD4), gamma-delta T cells, resting NK cells, activated NK cells, monocytes, M0 macrophages, M1 macrophages, M2 macrophages, tissue associated macrophages (TAMs) (e.g., a marker for TAM includes, without limitation, CD163, MSR1, and MRC1), resting dendritic cells, activated dendritic cells, resting mast cells, activated mast cells, eosinophils, neutrophils and any combinations thereof.

In some embodiments, a monocyte marker can include, without limitation, CD14, CD16, and FCN1 or any combination thereof. In some embodiments, a T cell marker includes, without limitation, CD3D, CD3E, and CD4 or any combination thereof. In some embodiments, individual T cell markers include, without limitation, CD4, CD8, TIGIT, and LAG3. In some embodiments, a B cell marker includes, without limitation, CD19, CD79A, and CD79B or any combination thereof. In some embodiments, a cancer marker can include, without limitation, BRCA1 and BRCA2 or any combination thereof.

In some embodiments, the method also includes identifying the ratio of one or more TILs to one or more TIBs in the biological sample. One skilled in the art would appreciate the ratio to cover the inverse ratio of TIB to TIL. The ratio of TILs to TIBs can include a ratio for a region of interest within the biological sample. In some cases, the region of interest can encompass the biological sample. One or more ratios of TILs to TIBs can be calculated for a biological sample. For example, each of two or more regions of interest each include a ratio of TILs to TIBs. In some embodiments, the ratio of TILs to TIBs can linked to a prognostic outcome.

In some embodiments, the method also includes identifying the ratio of one or more tumor infiltrating T cells to one or more TIBs in the biological sample. One skilled in the art would appreciate the ratio to cover the inverse ratio of TIB to tumor infiltrating T cells. The ratio of tumor infiltrating T cells to TIBs can include a ratio for a region of interest within the biological sample. In some cases, the region of interest can encompass the biological sample. One or more ratios of tumor infiltrating T cells to TIBs can be calculated for a biological sample. For example, each of two or more regions of interest each include a ratio of tumor infiltrating T cells to TIBs. In some embodiments, the ratio of tumor infiltrating T cells to TIBs can linked to a prognostic outcome.

In some embodiments, the method also includes identifying the ratio of one or more TILs and/or one or more TIBs to one or more stromal regions and/or one cancerous regions in the biological sample. One skilled in the art would appreciate the ratio to cover the inverse ratio of stromal region and/or cancerous region to TIL and/or TIB. The ratio of TILs and/or TIBs to stromal region and/or cancerous region can include a ratio for a region of interest within the biological sample. In some cases, the region of interest can encompass the biological sample. In some cases, one or more ratios of TILs and/or TIBs to stromal regions and/or cancerous regions can be calculated for a biological sample. For example, each of two or more regions of interest each include a ratio of TILs and/or TIBs to stromal regions and/or cancerous regions. In some embodiments, the ratio of TILs and/or TIBs to stromal regions and/or cancerous regions can be linked to a prognostic outcome.

In some embodiments, the method for determining immune cell infiltration includes identifying the abundance and/or spatial location of an analyte associated with the cancerous region. Non-limiting examples of analytes associated with a cancerous region include: SCGB2A1, MK167, BRCA1, BRCA2, PIKCD, CALML6, MYC, TP53, PALB2, RAD51, and/or MSH2. Non-limiting examples of analytes associated with an immune infiltrating cell can also include byproducts, precursors, and degradation products of such analytes thereof, and any combination of such analytes and byproducts, precursors, and degradation products thereof. Additional non-limiting examples of analytes associated with a cancerous region include (in addition to/in combination with the previously listed markers in this paragraph) SCGB2A1, MKI67, BRCA1, BRCA2, PIK3CD, and/or CALML6. Non-limiting examples of analytes associated with an immune infiltrating cell can also include byproducts, precursors, and degradation products of such analytes thereof, and any combination of such analytes and byproducts, precursors, and degradation products thereof. In some instances, additional non-limiting examples of analytes associated with a cancerous region include (in addition to/in combination with the previously listed markers in this paragraph) PRKCI, VTCN1, MECOM, TOP2A, SHDH, XPO1, TFRC, FUT8, SOX17, PBX1, EIF42, and/or WT1. Non-limiting examples of analytes associated with an immune infiltrating cell can also include byproducts, precursors, and degradation products of such analytes thereof, and any combination of such analytes and byproducts, precursors, and degradation products thereof. In some instances, additional non-limiting examples of analytes associated with a cancerous region include (in addition to/in combination with the previously listed markers in this paragraph) VTCN1, MECOM, TOP2A, XPO1, FUT8, SOX17, PBX1, EIF42, and WT1. Non-limiting examples of analytes associated with an immune infiltrating cell can also include byproducts, precursors, and degradation products of such analytes thereof, and any combination of such analytes and byproducts, precursors, and degradation products thereof.

Other non-limiting examples of such analytes are described in https://www.cancer.gov/about-cancer/diagnosis-staging/diagnosis/tumor-markers-list, which is hereby incorporated by reference in its entirety. In some embodiments, the analyte associated with the cancerous region is selected from the group comprising an analyte from the AKT pathway, an analyte from the JAK-STAT pathway, and an analyte from the Notch pathway.

In some embodiments, the method for determining immune cell infiltration includes the identifying abundance and/or spatial location of an analyte associated with the stromal region. Non-limiting examples of analytes associated with a stromal region include: VIM, EPCAM, FAP, and CDH1. Non-limiting examples of analytes associated with an immune infiltrating cell can also include byproducts, precursors, and degradation products of such analytes thereof, and any combination of such analytes and byproducts, precursors, and degradation products thereof. Additional non-limiting examples of analytes associated with a stromal region include: FAP, VCAN, ACTA2, and PDGFRB. Non-limiting examples of analytes associated with an immune infiltrating cell can also include byproducts, precursors, and degradation products of such analytes thereof, and any combination of such analytes and byproducts, precursors, and degradation products thereof.

In some embodiments, the method includes identifying expression of epithelial cell adhesion molecule (EPCAM; NCBI Gene ID: 4072) and vimentin (VIM; NCBI Gene ID: 7431). In some embodiments, the method includes identifying up-regulation (e.g., over expression) of EPCAM and down-regulation (e.g., under expression) of VIM compared to expression of the same genes in other areas of the same biological sample. In some embodiments, the method includes identifying up-regulation (e.g., over expression) of VIM and down-regulation (e.g., under expression) of EPCAM compared to expression of the same genes in other areas of the same biological sample. In some instances, any one or combination or cancerous or stromal biomarkers disclosed herein can be determined using spatial methods disclosed herein at locations where EPCAM or VIM is expressed.

In some embodiments, the method includes identifying expression of epithelial cell adhesion molecule (EPCAM; NCBI Gene ID: 4072) and fibroblast activation protein (FAP; NCBI Gene ID: 2191). In some embodiments, the method includes identifying up-regulation (e.g., over expression) of EPCAM and down-regulation (i.e., under expression) of FAP compared to expression of the same genes in other areas of the same biological sample. In some embodiments, the method includes identifying up-regulation (e.g., over expression) of FAP and down-regulation (e.g., under expression) of EPCAM compared to expression of the same genes in other areas of the same biological sample. In some instances, any one or combination or cancerous or stromal biomarkers disclosed herein can be determined using spatial methods disclosed herein at locations where EPCAM or FAP is expressed.

In some embodiments, the method includes identifying expression of VIM, CDH1, and FAP. In some instances, any one or combination or cancerous or stromal biomarkers disclosed herein can be determined using spatial methods disclosed herein at locations where EPCAM, CDH1, or VIM is expressed.

In some embodiments, the method includes identifying expression of protein tyrosine phosphatase receptor type C (CD45; NCBI Gene ID 5788). In some embodiments, the method includes up-regulation (e.g., over expression) of CD45 polypeptide. In some instances, the method includes down-regulation (e.g., under expression) of CD45 polypeptide. In some embodiments, the method includes identifying human keratin proteins (e.g., using a pan cytokeratin antibody or antigen-binding fragment). In some cases, detecting keratins using a pan cytokeratin antibody or antigen-binding fragment can be used to differentiate epithelial tumors from non-epithelial tumors. Non-limiting examples of keratin proteins that can be recognized by include: Type I or LMW cytokeratin, basic (Type II or HMW) cytokeratin (e.g., CK1, CK3, CK4, CK5, CK6, CK8, CK10, CK14, CK15, CK16, and CK19). CD45 is a pan leukocyte marker that resides in stroma of tumor sections, and can be used as a marker for tumor stroma. In some embodiments, the method for determining immune cell infiltration includes identifying abundance and/or spatial location of an analyte associated with a tumor stromal region. In some embodiments, the analyte is CD45.

In some embodiments, the method further includes contacting the biological sample with one or more stains. In some embodiments, the one or more stains comprise a histology stain (e.g., any of the histology stains described herein or known in the art). In some embodiments, the one or more stains comprises hematoxylin and eosin. In some embodiments, the one or more stains comprise one or more optical labels (e.g., any of the optical labels described herein). In some embodiments, the one or more optical labels are selected from the group consisting of: fluorescent, radioactive, chemiluminescent, calorimetric, or colorimetric labels.

In some embodiments, the method further includes identifying one or more cancerous regions in the biological sample using the one or more stains of the biological sample. In some embodiments, the method further includes identifying one or more stromal regions within the one or more cancerous regions using the one or more stains of the biological sample.

In some embodiments, the method further comprises determining a prognosis of the cancer in a subject based on the abundance and/or location of the TIL in the biological sample.

In some embodiments, the method further includes scoring or determining the severity of the cancer in the subject based on the abundance and/or location of the TIL in the biological sample.

(e) Therapeutic Methods

In some embodiments, the methods can further include selecting a treatment for the subject. In some embodiments, the methods can further include administering a treatment of cancer to the subject. In some embodiments, a treatment of cancer can be a treatment that reduces the rate of progression of cancer. In some embodiments, a treatment of cancer can include surgery, radiation therapy, chemotherapy, targeted drug therapy, and tumor treating fields (TTF) therapy.

In some instances, the methods disclosed herein include treating a subject having cancer with one or more therapeutic agents. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents, apoptotic agents, anti-tubulin agents, and other-agents (e.g., antibodies) to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva®), platelet derived growth factor inhibitors (e.g., Gleevec® (Imatinib Mesylate)), a COX-2 inhibitor (e.g., celecoxib), interferons, CTLA-4 inhibitors (e.g., anti-CTLA antibody ipilimumab (YERVOY®)), PD-1 inhibitors (e.g., anti-PD-1 antibodies, BMS-936558), PD-L1 inhibitors (e.g., anti-PD-L1 antibodies, MPDL3280A), PD-L2 inhibitors (e.g., anti-PD-L2 antibodies), TIM3 inhibitors (e.g., anti-TIM3 antibodies), cytokines, antagonists (e.g., neutralizing antibodies) that bind to one or more of the following targets ErbB2, ErbB3, ErbB4, PDGFR-beta, BlyS, APRIL, BCMA, PD-1, PD-L1, PD-L2, CTLA-4, TIM3, or VEGF receptor(s), TRAIL/Apo2, and other bioactive and organic chemical agents, etc. In some instances, the therapy or treatment includes surgery, chemotherapeutic agents, growth inhibitory agents, cytotoxic agents, agents used in radiation therapy, anti-angiogenesis agents, cancer immunotherapeutic agents, apoptotic agents, anti-tubulin agents, or a combination thereof.

In some instances, chemotherapeutic agents are provided as a therapy to a subject having cancer. Nonlimiting exemplary chemotherapeutic agents include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

In some embodiments, radiation therapy is administered locally to a tumor lesion to enhance the local immunogenicity of a subject's tumor (e.g., adjuvinating radiation) and/or to kill tumor cells (e.g., ablative radiation). In some instances, radiation therapy is administered systemically to a subject. In some instances, the radiation therapy is tomotherapy, stereotactic radiation, intensity-modulated radiation therapy (IMRT), hypofractionated radiotherapy, hypoxia-guided radiotherapy, and/or proton therapy. In some instances, radiation is followed by administration of a second therapy (e.g., chemotherapy, immunotherapy). In some instances, radiation is provided concurrently with administration of a second therapy (e.g., chemotherapy, immunotherapy).

In some instances, any of the above therapeutic agents are provided before, substantially contemporaneous with, or after other modes of treatment, for example, surgery, chemotherapy, radiation therapy, or the administration of a biologic, such as another therapeutic antibody. In some embodiments, the cancer has recurred or progressed following a therapy selected from surgery, chemotherapy, and radiation therapy, or a combination thereof.

In some instances, for treatment of cancer, as discussed herein, the antibodies are administered in conjunction with one or more additional anti-cancer agents, such as the chemotherapeutic agent, growth inhibitory agent, anti-angiogenesis agent and/or anti-neoplastic composition. Nonlimiting examples of chemotherapeutic agent, growth inhibitory agent, anti-angiogenesis agent, anti-cancer agent and anti-neoplastic composition.

In some embodiments, the methods can further include updating the subject's clinical record with the diagnosis of cancer. In some embodiments, the methods can further include enrolling the subject in a clinical trial. In some embodiments, the methods can further include informing the subject's family of the diagnosis. In some embodiments, the methods can further include assessing or referring the subject for enrollment in a supportive care plan or care facility. In some embodiments, the methods can further include monitoring the subject more frequently.

In some embodiments, the methods can further comprise monitoring the identified subject for the development of symptoms of cancer. In some embodiments, the methods can further include recording in the identified subject's clinical record that the subject has an increased likelihood of developing cancer. In some embodiments, the methods can further include notifying the subject's family that the subject has an increased likelihood or susceptibility of developing cancer.

In some embodiments, the methods can further include administering to the subject a treatment for decreasing the rate of progression or decreasing the likelihood of developing cancer. In some embodiments, a treatment of cancer can include surgery, radiation therapy, chemotherapy, surgery, radiation therapy, chemotherapy, targeted drug therapy, and tumor treating fields (TTF) therapy. In some embodiments, the subject can be tested for the presence of genetic mutations known to be associated with risk for cancer.

In some embodiments, the methods can further include performing one or more tests to further determine the subject's risk of developing cancer. Non-limiting examples of more tests to further determine the subject's risk of developing cancer include, detecting a genetic mutation associated with cancer (e.g., a mutation associated with neurofibromatosis type 1, Turcot syndrome, or Li Fraumeni syndrome), and determining the levels of other biomarkers (e.g., in brain tissue, cerebrospinal fluid, or in blood or a component thereof) indicative an increased risk of developing cancer are indicative of an increased risk of developing cancer.

In some embodiments, the methods can further include updating the subject's clinical record to indicate an increased risk of developing cancer. In some embodiments, the methods can further include enrolling the subject in a clinical trial (e.g., for the early treatment and/or prevention of cancer). In some embodiments, the methods can further include informing the subject's family of the subject's likelihood of developing cancer. In some embodiments, the methods can further include monitoring the subject more frequently.

In certain embodiments, the cancer treated in accordance with the methods described herein includes but is not limited to prostate cancer, breast cancer, lung cancer, colorectal cancer, melanoma, bronchial cancer, bladder cancer, brain or central nervous system cancer, peripheral nervous system cancer, uterine or endometrial cancer, cancer of the oral cavity or pharynx, non-Hodgkin's lymphoma, thyroid cancer, kidney cancer, biliary tract cancer, small bowel or appendix cancer, salivary gland cancer, thyroid gland cancer, adrenal gland cancer, squamous cell cancer, mesothelioma, osteocarcinoma, thyoma/thymic carcinoma, glioblastoma, myelodysplastic syndrome, soft tissue sarcoma, DIPG, adenocarcinoma, osteosarcoma, chondrosarcoma, leukemia, or pancreatic cancer. In some embodiments, the cancer treated in accordance with the methods described herein includes a carcinoma (e.g., an adenocarcinoma), lymphoma, blastoma, melanoma, sarcoma or leukemia. In certain embodiments, the cancer treated in accordance with the methods described herein includes squamous cell cancer, small-cell lung cancer, non-small cell lung cancer, gastrointestinal cancer, Hodgkin's lymphoma, non-Hodgkin's lymphoma, pancreatic cancer, glioblastoma, glioma, cervical cancer, ovarian cancer, liver cancer (e.g., hepatic carcinoma and hepatoma), bladder cancer, breast cancer, inflammatory breast cancer, Merkel cell carcinoma, colon cancer, colorectal cancer, stomach cancer, urinary bladder cancer, endometrial carcinoma, myeloma (e.g., multiple myeloma), salivary gland, carcinoma, kidney cancer (e.g., renal cell carcinoma and Wilms' tumors), basal cell carcinoma, melanoma, prostate cancer, vulval cancer, thyroid cancer, testicular cancer, esophageal cancer, serous adenocarcinoma or various types of head and neck cancer. In certain embodiments, the cancer treated in accordance with the methods described herein includes desmoplastic melanoma, inflammatory breast cancer, thymoma, rectal cancer, anal cancer, or surgically treatable or non-surgically treatable brain stem glioma.

(f) Kits and Systems

In some embodiments, also provided herein are kits that include one or more reagents to detect a level of one or more of any of the cells and/or biomarkers associated with cancerous regions and one or more stromal regions as described herein. In some embodiments, also provided herein are kits that include one or more reagents to detect a level of one or more of any of the cells and/or biomarkers associated with cancerous regions and one or more stromal regions as described herein.

In some embodiments, reagents can include one or more antibodies (and/or antigen-binding antibody fragments), labeled hybridization probes, and primers. For example, in some embodiments, an antibody (and/or antigen-binding antibody fragment) can be used for visualizing one or more features of a tissue sample (e.g., by using immunofluorescence or immunohistochemistry). In some embodiments, an antibody (and/or antigen-binding antibody fragment) can be an analyte binding moiety, for example, as part of an analyte capture agent. For example, in some embodiments, a kit can include an anti-PMCH antibody, such as Product No. HPA046055 (Atlas Antibodies), Cat. Nos. PA5-25442, PA5-84521, PA5-83802 (ThermoFisher Scientific), or Product No. AV13054 (MilliporeSigma). Other useful commercially available antibodies will be apparent to one skilled in the art.

In some embodiments, labeled hybridization probes can be used for in situ sequencing of one or more biomarkers and/or candidate biomarkers. In some embodiments, primers can be used for amplification (e.g., clonal amplification) of a captured oligonucleotide analyte.

In some embodiments, a kit can further include instructions for performing any of the methods or steps provided herein. In some embodiments, a kit can include a substrate with one or more capture probes comprising a spatial barcode and a capture domain that binds to a biological analyte from a tissue sample, and reagents to detect a biological analyte, wherein the biological analyte is any of the biomarkers of this disclosure. In some embodiments, the kit further includes but is not limited to one or more antibodies (and/or antigen-binding antibody fragments), labeled hybridization probes, primers, or any combination thereof for visualizing one or more features of a tissue sample.

Also described herein are systems that include one or more storage elements (e.g., one or more storage devices) and one or more processors. The storage element can store a dataset of multiple biological samples. For each biological sample, the dataset can include analyte data for multiple analytes that are captured at multiple spatial locations of a reference biological sample. The dataset can further include image data of the biological sample. Additionally, the dataset can include registration data of the imaged data that link to the analyte data according to the spatial locations of the reference biological sample. The biological sample can include one or more cancerous regions in the reference biological sample, one or more stromal regions within the one or more cancerous regions, and/or one or more tumor infiltrating lymphocytes (TILs). The processor can process the dataset through a machine learning module to train the machine learning module, so as to determine immune cell infiltration in a biological sample.

### EXAMPLES

### Example 1—Detection of Tumor Infiltrating Lymphocytes in a Biological Sample

This example provides an exemplary method of determining immune cell infiltration in cancer stroma of a test biological sample. In a non-limiting example, a test biological sample is contacted with a substrate including a plurality of capture probes, wherein a capture probe of the plurality of capture probes includes a spatial barcode. The biological sample is permeabilized and analytes from the test biological sample are hybridized to the capture probe. The capture probe is extended, and a second strand is generated that includes a sequence of the analyte or a complement thereof.

All or a part of a sequence corresponding to the analyte, or a complement thereof, and (ii) all or a part of a sequence corresponding to the spatial barcode, or a complement thereof, determined, and the determined sequence of (i) and (ii) is used to identify the abundance and/or spatial location of the analyte in the test biological sample.

A machine learning module is trained on a dataset that includes a plurality of biological samples. The machine learning module is trained on data where a biological sample includes the following data: (i) analyte data for a plurality of analytes captured from a plurality of spatial locations in the biological sample; (ii) image data comprising images of the plurality of spatial locations of the biological sample; and (iii) registration data linking the analyte data to the image data. The plurality of biological samples includes reference biological samples, where a reference biological sample includes: (1) one or more cancerous regions in the reference biological sample, (2) zero or one or more stromal regions within the one or more cancerous regions, and (3) zero or one or more immune infiltrating cells. The machine learning module is trained with the dataset, according to the process shown in FIG. 7, resulting in a trained machine learning module. The trained machine learning module is then used to determine immune cell infiltration in a biological sample based at least in part on the abundance and/or location of an analyte in the test biological sample.

### Example 2—Determination of Infiltrating Immune Cells Using Gene Clusters

This example provides an exemplary method of determining immune cell infiltration in cancer stroma of a test biological sample. Cancerous regions within the biological sample are identified using a tissue detection machine learning module as described in Example 1. Cancerous regions can also be identified by eye by a pathologist or by determining cancer gene expression signatures (e.g., using any of the methods described herein or known in the art).

Next, stromal regions are identified within the cancer regions using a tissue detection machine learning module, by eye by a pathologist, or by determining stromal gene expression signatures (e.g., using any of the methods described herein or known in the art).

The test biological sample is contacted with a substrate including a plurality of capture probes, wherein a capture probe of the plurality of capture probes includes a spatial barcode. The biological sample is permeabilized and an analytes from the test biological sample are hybridized to the capture probes. The capture probe is extended, and a second strand is generated that includes a sequence of the analyte or a complement thereof.

All or a part of a sequence corresponding to the analyte, or a complement thereof, and (ii) all or a part of a sequence corresponding to the spatial barcode, or a complement thereof, is determined, and the determined sequence identifies a gene cluster associated with an immune infiltrating cell. An abundance of infiltrating immune cells in stromal cancer regions is calculated as a percentage (0-100%) of the area biological sample. The abundance of immune infiltrating cells in stromal cancer regions is predictive of clinical outcome.

### Example 3—Determining Location of Immune Cell Infiltrates, Cancer Biomarkers, and Stromal Compartment Biomarkers in Ovarian Adenocarcinoma

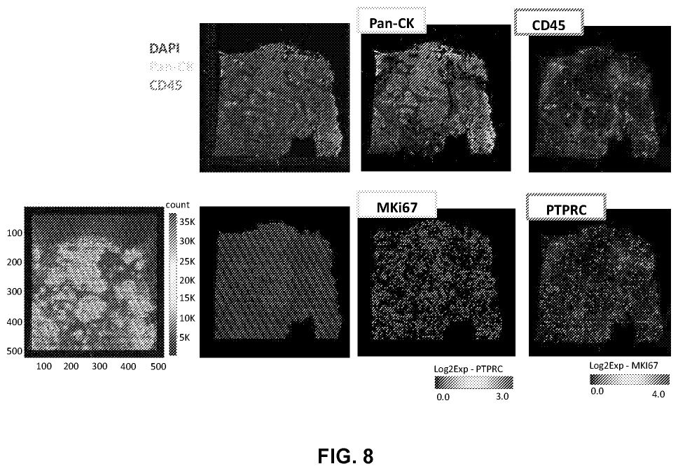

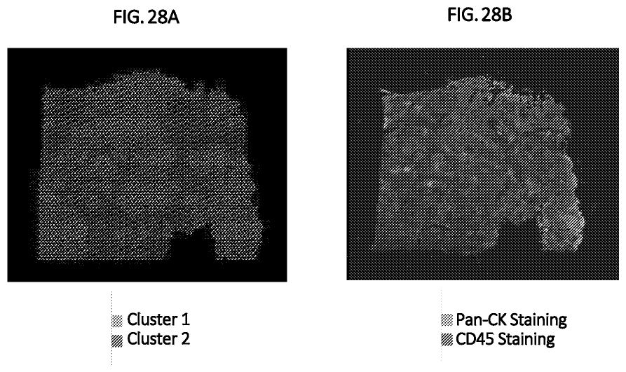

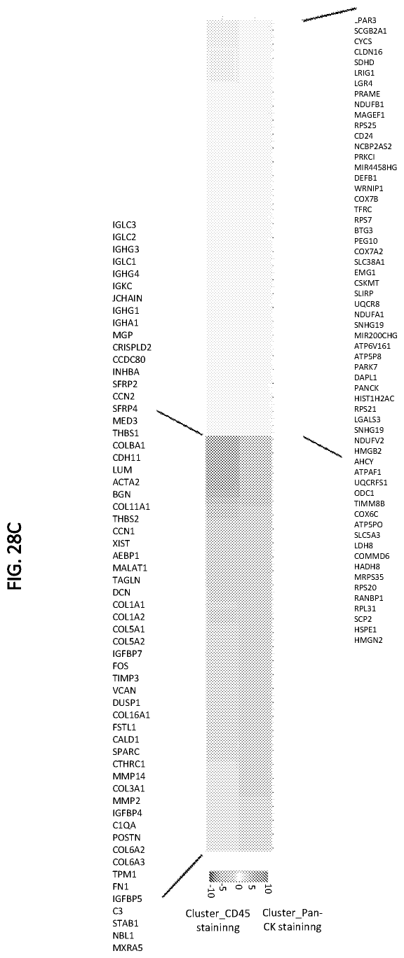

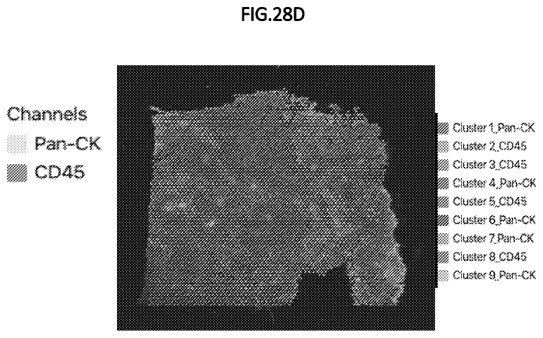

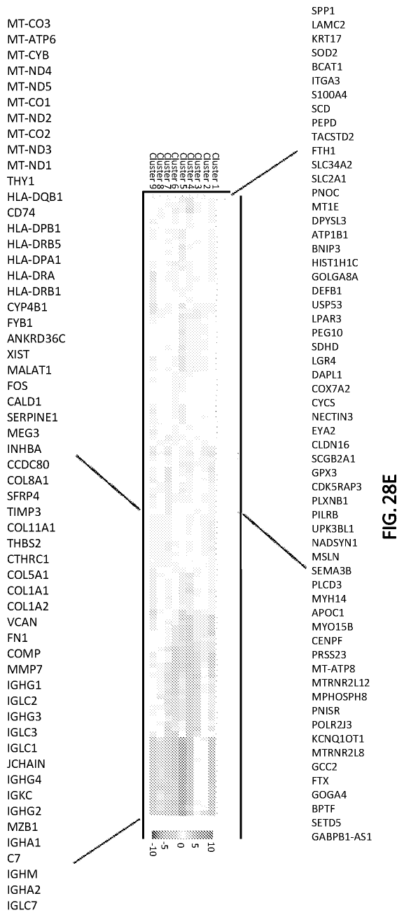

This example provides an exemplary method for determining immune cell infiltration in cancer stroma of a patient having cancer using immunofluorescence and spatial profiling. The biological sample was an endometrial adenocarcinoma of the ovary. Based on the AJCC/UICC staging, the tumor was T1NOMO (https://www.cancer.gov/about-cancer/diagnosis-staging/staging) with a AJCC/UICC Stage group of I. Ovarian tissue sections were stained with a pancytokeratin (Pan-CK) antibody (Biolegend) and/or with an antibody against CD45 (Biolegend), and DAPI (FIG. 8, top panel; see also FIG. 28B). Pan—CK was used to identify tumor compartments and CD45 was used to identify tumor stromal compartments in the tissue section. Tissue sections were also profiled for gene expression using the 10× Genomics Visium Spatial Gene Expression platform (FIG. 8, bottom panel). Spatial gene expression data was subjected to unsupervised k-means clustering into two clusters. Cluster 1 correlated strongly with the Pan-CK immunostained (tumor) compartment, while Cluster 2 correlated strongly with the CD45 immunostained (stromal) compartment. See FIG. 28A and FIG. 28B. Gene expression was analyzed. FIG. 28C shows a heatmap of differentially expressed genes in Cluster 1 (correlating with the tumor compartments positive for Pan-CK immunostaining) (top row of heat map) and Cluster 2 (stromal compartments positive for CD45 immunostaining) (bottom row of heat map). Tables 1-4 lists the top 20 up-regulated and top 20 down-regulated genes from Cluster 1 and Cluster 2.

TABLE 1
Top 20 Up-regulated genes for cluster 1

| | | Cluster 1 | Cluster 1 Log2 | Cluster 1 |
|---|---|---|---|---|
| FeatureID | FeatureName | Average | Fold Change | P-Value |
| ENSG00000124939 | SCGB2A1 | 3.08382728 | 0.967697372 | 0.001362318 |
| ENSG00000171517 | LPAR3 | 1.159438958 | 0.946785953 | 0.00281696 |
| ENSG00000113946 | CLDN16 | 3.08783225 | 0.921830862 | 0.002504996 |
| ENSG00000172115 | CYCS | 2.062559804 | 0.906346555 | 0.003244383 |
| ENSG00000144749 | LRIG1 | 2.439694525 | 0.838234921 | 0.007818152 |
| ENSG00000204370 | SDHD | 1.854301338 | 0.833447998 | 0.008615292 |
| ENSG00000185686 | PRAME | 1.527228748 | 0.828519803 | 0.011646171 |
| ENSG00000205213 | LGR4 | 1.090686964 | 0.826501656 | 0.013107306 |
| ENSG00000183648 | NDUFB1 | 1.641036659 | 0.805396715 | 0.014919809 |
| ENSG00000177383 | MAGEF1 | 1.770196958 | 0.802940144 | 0.015113184 |
| ENSG00000270170 | NCBP2AS2 | 1.080007043 | 0.788679898 | 0.020099007 |
| ENSG00000272398 | CD24 | 6.859846959 | 0.787412653 | 0.013302791 |
| ENSG00000118181 | RPS25 | 25.48062352 | 0.786877382 | 0.012953038 |
| ENSG00000163558 | PRKCI | 3.009401578 | 0.785314727 | 0.014492846 |
| ENSG00000247516 | MIR4458HG | 1.144420318 | 0.783849451 | 0.020789037 |
| ENSG00000164825 | DEFB1 | 2.780450765 | 0.770572511 | 0.018021273 |
| ENSG00000124535 | WRNIP1 | 1.1400816 | 0.766175825 | 0.025310118 |
| ENSG00000171863 | RPS7 | 14.29674342 | 0.760533065 | 0.017714594 |
| ENSG00000072274 | TFRC | 2.271485765 | 0.75682611 | 0.020679768 |
| ENSG00000131174 | COX7B | 2.049877397 | 0.756587137 | 0.021104042 |

TABLE 2
Top 20 Up-regulated genes for Cluster 2

| | | Cluster 2 | Cluster 2 Log2 | Cluster 2 |
|---|---|---|---|---|
| FeatureID | FeatureName | Average | Fold Change | P-Value |
| ENSG00000211679 | IGLC3 | 2.181536186 | 5.708114681 | 4.85E−83 |
| ENSG00000211675 | IGLC1 | 8.506974354 | 5.543888175 | 6.21E−91 |
| ENSG00000211897 | IGHG3 | 24.65892818 | 5.456807905 | 6.38E−105 |
| ENSG00000211677 | IGLC2 | 6.752481503 | 5.436188028 | 8.43E−84 |
| ENSG00000211892 | IGHG4 | 103.5275054 | 5.300646075 | 4.89E−104 |
| ENSG00000132465 | JCHAIN | 2.349868082 | 5.241922025 | 2.43E−88 |
| ENSG00000211592 | IGKC | 126.1495047 | 5.223886818 | 1.80E−103 |
| ENSG00000211896 | IGHG1 | 9.031175696 | 5.100850085 | 2.65E−94 |
| ENSG00000211895 | IGHA1 | 14.04611051 | 5.013508717 | 7.67E−82 |
| ENSG00000111341 | MGP | 1.622312772 | 4.008459193 | 2.50E−60 |
| ENSG00000091986 | CCDC80 | 2.940724335 | 3.458874887 | 9.37E−56 |
| ENSG00000103196 | CRISPLD2 | 1.272092048 | 3.376655134 | 2.00E−48 |
| ENSG00000145423 | SFRP2 | 2.256099308 | 3.179328634 | 2.22E−44 |
| ENSG00000122641 | INHBA | 1.921695004 | 3.173622294 | 6.85E−46 |
| ENSG00000118523 | CCN2 | 3.337264574 | 3.135945463 | 2.02E−47 |
| ENSG00000214548 | MEG3 | 1.380547498 | 3.125485026 | 8.63E−43 |
| ENSG00000106483 | SFRP4 | 1.008861633 | 3.115067381 | 8.23E−37 |
| ENSG00000144810 | COL8A1 | 1.855040092 | 3.028625363 | 1.31E−41 |
| ENSG00000137801 | THBS1 | 3.693134019 | 3.026889501 | 9.93E−44 |
| ENSG00000140937 | CDH11 | 1.095851942 | 2.893444512 | 1.06E−35 |

TABLE 3
Top 20 Down-regulated genes for Cluster 1

| | | Cluster 1 | Cluster 1 Log2 | Cluster 1 |
|---|---|---|---|---|
| FeatureID | FeatureName | Average | Fold Change | P-Value |
| ENSG00000211679 | IGLC3 | 0.041384695 | −5.708114681 | 4.85E−83 |
| ENSG00000211675 | IGLC1 | 0.18189241 | −5.543888175 | 6.21E−91 |
| ENSG00000211897 | IGHG3 | 0.560695869 | −5.456807905 | 6.38E−105 |
| ENSG00000211677 | IGLC2 | 0.155526354 | −5.436188028 | 8.43E−84 |
| ENSG00000211892 | IGHG4 | 2.624256915 | −5.300646075 | 4.89E−104 |
| ENSG00000132465 | JCHAIN | 0.061743295 | −5.241922025 | 2.43E−88 |
| ENSG00000211592 | IGKC | 3.372518903 | −5.223886818 | 1.80E−103 |
| ENSG00000211896 | IGHG1 | 0.262659315 | −5.100850085 | 2.65E−94 |
| ENSG00000211895 | IGHA1 | 0.434205551 | −5.013508717 | 7.67E−82 |
| ENSG00000111341 | MGP | 0.10045801 | −4.008459193 | 2.50E−60 |
| ENSG00000091986 | CCDC80 | 0.266998033 | −3.458874887 | 9.37E−56 |
| ENSG00000103196 | CRISPLD2 | 0.1221516 | −3.376655134 | 2.00E−48 |
| ENSG00000145423 | SFRP2 | 0.248641918 | −3.179328634 | 2.22E−44 |
| ENSG00000122641 | INHBA | 0.212597184 | −3.173622294 | 6.85E−46 |
| ENSG00000118523 | CCN2 | 0.379137207 | −3.135945463 | 2.02E−47 |
| ENSG00000214548 | MEG3 | 0.157862587 | −3.125485026 | 8.63E−43 |
| ENSG00000106483 | SFRP4 | 0.116144144 | −3.115067381 | 8.23E−37 |
| ENSG00000144810 | COL8A1 | 0.226948328 | −3.028625363 | 1.31E−41 |
| ENSG00000137801 | THBS1 | 0.452561666 | −3.026889501 | 9.93E−44 |
| ENSG00000140937 | CDH11 | 0.147182666 | −2.893444512 | 1.06E−35 |

TABLE 4
Top 20 Down-regulated genes for Cluster 2

| | | Cluster 2 | Cluster 2 Log2 | Cluster 2 |
|---|---|---|---|---|
| FeatureID | FeatureName | Average | Fold Change | P-Value |
| ENSG00000124939 | SCGB2A1 | 1.577123001 | −0.967697372 | 0.001362318 |
| ENSG00000171517 | LPAR3 | 0.601023952 | −0.946785953 | 0.00281696 |
| ENSG00000113946 | CLDN16 | 1.630220982 | −0.921830862 | 0.002504996 |
| ENSG00000172115 | CYCS | 1.100370919 | −0.906346555 | 0.003244383 |
| ENSG00000144749 | LRIG1 | 1.364731078 | −0.838234921 | 0.007818152 |
| ENSG00000204370 | SDHD | 1.040494473 | −0.833447998 | 0.008615292 |
| ENSG00000185686 | PRAME | 0.85973539 | −0.828519803 | 0.011646171 |
| ENSG00000205213 | LGR4 | 0.614580883 | −0.826501656 | 0.013107306 |
| ENSG00000183648 | NDUFB1 | 0.938817489 | −0.805396715 | 0.014919809 |
| ENSG00000177383 | MAGEF1 | 1.014510355 | −0.802940144 | 0.015113184 |
| ENSG00000270170 | NCBP2AS2 | 0.624748581 | −0.788679898 | 0.020099007 |
| ENSG00000272398 | CD24 | 3.976699831 | −0.787412653 | 0.013302791 |
| ENSG00000118181 | RPS25 | 14.77931454 | −0.786877382 | 0.012953038 |
| ENSG00000163558 | PRKCI | 1.746584642 | −0.785314727 | 0.014492846 |
| ENSG00000247516 | MIR4458HG | 0.664289631 | −0.783849451 | 0.020789037 |
| ENSG00000164825 | DEFB1 | 1.630220982 | −0.770572511 | 0.018021273 |
| ENSG00000124535 | WRNIP1 | 0.669938352 | −0.766175825 | 0.025310118 |
| ENSG00000171863 | RPS7 | 8.444838419 | −0.760533065 | 0.017714594 |
| ENSG00000072274 | TFRC | 1.344395681 | −0.75682611 | 0.020679768 |
| ENSG00000131174 | COX7B | 1.213345346 | −0.756587137 | 0.021104042 |

Spatial gene expression data was further subjected to unsupervised graph-based clustering into nine clusters. As shown in FIG. 28D, clusters 1, 4, 6, 7, and 9 were correlated with tumor compartments expressing Pan-CK, and clusters 2, 3, 5, and 8 were correlated with stromal compartments expressing CD45. FIG. 28E is a heatmap that shows relative gene dysregulation of various genes in each cluster. Tables 5 and 6 list the top 20 up-regulated and top 20 down-regulated genes for each cluster (1-9).

TABLE 5
Top 20 Up-regulated genes for each cluster (1-9)

| | | Cluster 1 | Cluster 1 Log2 | Cluster 1 |
|---|---|---|---|---|
| FeatureID | FeatureName | Average | Fold Change | P-Value |
| ENSG00000099194 | SCD | 2.893387183 | 0.771453892 | 0.014491557 |
| ENSG00000118785 | SPP1 | 5.22763427 | 0.74990077 | 0.027675295 |
| ENSG00000112096 | SOD2 | 2.077470604 | 0.749765561 | 0.024344716 |
| ENSG00000164825 | DEFB1 | 3.554865732 | 0.744292769 | 0.021892389 |
| ENSG00000196154 | S100A4 | 12.49924656 | 0.688116761 | 0.043957922 |
| ENSG00000184292 | TACSTD2 | 2.423560647 | 0.659983218 | 0.070839409 |
| ENSG00000157765 | SLC34A2 | 4.182851696 | 0.636119379 | 0.090621735 |
| ENSG00000167996 | FTH1 | 55.62467083 | 0.635150426 | 0.083867563 |
| ENSG00000168081 | PNOC | 1.498793168 | 0.628008889 | 0.112747926 |
| ENSG00000058085 | LAMC2 | 0.946165519 | 0.62129369 | 0.140036923 |
| ENSG00000128422 | KRT17 | 0.788936441 | 0.620590335 | 0.134252195 |
| ENSG00000117394 | SLC2A1 | 1.772315943 | 0.585363461 | 0.181644019 |
| ENSG00000124299 | PEPD | 1.038270127 | 0.583683243 | 0.190707901 |
| ENSG00000060982 | BCAT1 | 1.130374735 | 0.564317365 | 0.226846455 |
| ENSG00000123975 | CKS2 | 2.04211732 | 0.561159065 | 0.21270338 |
| ENSG00000196776 | CD47 | 2.596605668 | 0.553620038 | 0.22228105 |
| ENSG00000160213 | CSTB | 4.491727755 | 0.550541151 | 0.221048975 |
| ENSG00000102144 | PGK1 | 2.825471664 | 0.543604656 | 0.287115274 |
| ENSG00000227507 | LTB | 1.025245233 | 0.541944132 | 0.299171967 |
| ENSG00000124107 | SLPI | 4.958763243 | 0.536549149 | 0.247230884 |

| | | Cluster 2 | Cluster 2 Log2 | Cluster 2 |
|---|---|---|---|---|
| FeatureID | FeatureName | Average | Fold Change | P-Value |
| ENSG00000251562 | MALAT1 | 90.87636347 | 1.512578453 | 0.091563805 |
| ENSG00000269028 | MTRNR2L12 | 13.20530127 | 1.477322987 | 0.114587799 |
| ENSG00000082074 | FYB1 | 0.619066616 | 1.397033279 | 0.442212124 |
| ENSG00000122641 | INHBA | 1.281729473 | 1.193717386 | 0.953935932 |
| ENSG00000228253 | MT-ATP8 | 2.620134058 | 1.133519745 | 1 |
| ENSG00000229807 | XIST | 1.159659999 | 1.122914798 | 1 |
| ENSG00000106366 | SERPINE1 | 0.614706992 | 1.091018828 | 1 |
| ENSG00000122786 | CALD1 | 3.483339621 | 1.085440378 | 1 |
| ENSG00000211896 | IGHG1 | 4.490412779 | 1.076507131 | 1 |
| ENSG00000214548 | MEG3 | 0.850126691 | 1.050032952 | 1 |
| ENSG00000174501 | ANKRD36C | 0.640864736 | 1.048008153 | 1 |
| ENSG00000170345 | FOS | 1.586903157 | 1.016984592 | 1 |
| ENSG00000132424 | PNISR | 2.079540675 | 1.005637248 | 1 |
| ENSG00000196199 | MPHOSPH8 | 1.024511653 | 0.959031688 | 1 |
| ENSG00000134884 | ARGLU1 | 2.4283106 | 0.948132042 | 1 |
| ENSG00000118523 | CCN2 | 1.878997969 | 0.904290938 | 1 |
| ENSG00000101745 | ANKRD12 | 1.281729473 | 0.890106319 | 1 |
| ENSG00000198899 | MT-ATP6 | 73.98717987 | 0.860276412 | 1 |
| ENSG00000170776 | AKAP13 | 0.571110751 | 0.809838368 | 1 |
| ENSG00000084636 | COL16A1 | 0.90244218 | 0.804064101 | 1 |

| | | Cluster 3 | Cluster 3 Log2 | Cluster 3 |
|---|---|---|---|---|
| FeatureID | FeatureName | Average | Fold Change | P-Value |
| ENSG00000106483 | SFRP4 | 1.551568745 | 3.198562525 | 1.57E−27 |
| ENSG00000105664 | COMP | 1.480828529 | 2.809415982 | 1.89E−22 |
| ENSG00000060718 | COL11A1 | 2.242464858 | 2.809239538 | 1.05E−23 |
| ENSG00000100234 | TIMP3 | 3.765737517 | 2.656475505 | 9.39E−22 |
| ENSG00000186340 | THBS2 | 4.161882729 | 2.567943367 | 1.68E−20 |
| ENSG00000164932 | CTHRC1 | 1.688333164 | 2.510081738 | 9.55E−19 |
| ENSG00000214548 | MEG3 | 1.610518926 | 2.457686485 | 1.67E−17 |
| ENSG00000091986 | CCDC80 | 3.195099772 | 2.428689623 | 9.15E−18 |
| ENSG00000115414 | FN1 | 27.04870072 | 2.412131928 | 1.50E−18 |
| ENSG00000144810 | COL8A1 | 2.155218591 | 2.401424466 | 5.81E−17 |
| ENSG00000164692 | COL1A2 | 72.87421288 | 2.385592679 | 2.32E−18 |
| ENSG00000108821 | COL1A1 | 56.85155387 | 2.381447334 | 3.43E−18 |
| ENSG00000130635 | COL5A1 | 5.232418003 | 2.372412066 | 1.52E−17 |
| ENSG00000038427 | VCAN | 3.305926111 | 2.364809557 | 2.87E−17 |
| ENSG00000122641 | INHBA | 2.136354534 | 2.364388275 | 1.45E−16 |
| ENSG00000139329 | LUM | 5.246566046 | 2.35536426 | 6.21E−17 |
| ENSG00000204262 | COL5A2 | 3.732725416 | 2.305434182 | 3.40E−16 |
| ENSG00000113140 | SPARC | 25.31556542 | 2.303012512 | 1.00E−16 |
| ENSG00000103196 | CRISPLD2 | 1.313410017 | 2.278009021 | 2.42E−14 |
| ENSG00000166147 | FBN1 | 0.884252704 | 2.275060533 | 3.42E−13 |

| | | Cluster 4 | Cluster 4 Log2 | Cluster 4 |
|---|---|---|---|---|
| FeatureID | FeatureName | Average | Fold Change | P-Value |
| ENSG00000142973 | CYP4B1 | 1.589091282 | 1.312468397 | 6.50E−07 |
| ENSG00000171517 | LPAR3 | 1.782240499 | 1.087503797 | 8.86E−05 |
| ENSG00000124939 | SCGB2A1 | 4.630564336 | 1.039123176 | 0.000182082 |
| ENSG00000145390 | USP53 | 1.471195007 | 0.988911613 | 0.000813155 |
| ENSG00000150687 | PRSS23 | 1.511329909 | 0.835932057 | 0.008600477 |
| ENSG00000204370 | SDHD | 2.446974816 | 0.736702354 | 0.02929373 |
| ENSG00000163331 | DAPL1 | 2.411856776 | 0.713947245 | 0.040483693 |
| ENSG00000242265 | PEG10 | 1.79352844 | 0.699249845 | 0.050210179 |
| ENSG00000172115 | CYCS | 2.618802366 | 0.671985271 | 0.064980205 |
| ENSG00000064655 | EYA2 | 1.244181967 | 0.668744554 | 0.078194227 |
| ENSG00000113946 | CLDN16 | 3.876780705 | 0.651896679 | 0.07963741 |
| ENSG00000205213 | LGR4 | 1.370857752 | 0.636310694 | 0.10671302 |
| ENSG00000112695 | COX7A2 | 2.717885405 | 0.615656121 | 0.119106532 |
| ENSG00000177707 | NECTIN3 | 1.047270103 | 0.611221792 | 0.126378515 |
| ENSG00000163541 | SUCLG1 | 1.025948436 | 0.605656436 | 0.134032611 |
| ENSG00000198743 | SLC5A3 | 1.464923929 | 0.593505316 | 0.159749018 |
| ENSG00000125356 | NDUFA1 | 3.49675335 | 0.59231109 | 0.146911078 |
| ENSG00000247516 | MIR4458HG | 1.397196281 | 0.569306864 | 0.201614587 |
| ENSG00000152931 | PART1 | 1.044761672 | 0.565419942 | 0.190178142 |
| ENSG00000123472 | ATPAF1 | 1.284316869 | 0.562081463 | 0.215866804 |

| | | Cluster 5 | Cluster 5 Log2 | Cluster 5 |
|---|---|---|---|---|
| FeatureID | FeatureName | Average | Fold Change | P-Value |
| ENSG00000211685 | IGLC7 | 1.583218855 | 6.593191389 | 1.30E−13 |
| ENSG00000211899 | IGHM | 3.184847231 | 6.140018761 | 9.99E−39 |
| ENSG00000211897 | IGHG3 | 80.90524491 | 5.637703801 | 2.72E−76 |
| ENSG00000211677 | IGLC2 | 22.00858303 | 5.594624842 | 9.22E−62 |
| ENSG00000211892 | IGHG4 | 338.035635 | 5.564867399 | 1.95E−76 |
| ENSG00000211679 | IGLC3 | 6.958799152 | 5.545362864 | 6.55E−58 |
| ENSG00000132465 | JCHAIN | 7.667565733 | 5.543849131 | 2.80E−70 |
| ENSG00000211592 | IGKC | 409.6118549 | 5.511163358 | 2.48E−76 |
| ENSG00000112936 | C7 | 1.504978388 | 5.4964441 | 5.62E−54 |
| ENSG00000211893 | IGHG2 | 1.923795004 | 5.495033647 | 1.60E−45 |
| ENSG00000170476 | MZB1 | 2.287383055 | 5.444668864 | 1.74E−58 |
| ENSG00000211675 | IGLC1 | 25.99884683 | 5.288706192 | 2.89E−56 |
| ENSG00000211895 | IGHA1 | 44.16444224 | 5.282930677 | 1.74E−58 |
| ENSG00000211890 | IGHA2 | 2.22755211 | 5.054832431 | 7.22E−26 |
| ENSG00000211896 | IGHG1 | 26.02646112 | 4.924632334 | 2.89E−56 |
| ENSG00000111341 | MGP | 4.491923262 | 4.428196997 | 4.35E−44 |
| ENSG00000137077 | CCL21 | 1.104571294 | 4.087701006 | 2.63E−18 |
| ENSG00000115380 | EFEMP1 | 1.343895074 | 3.686957459 | 6.19E−25 |
| ENSG00000107562 | CXCL12 | 1.261052227 | 3.489922657 | 5.98E−23 |
| ENSG00000261371 | PECAM1 | 1.399123639 | 3.436598492 | 2.38E−22 |
| | | Cluster 6 | Cluster 6 Log2 | Cluster 6 |
| FeatureID | FeatureName | Average | Fold Change | P-Value |
| ENSG00000142973 | CYP4B1 | 1.440157355 | 0.889167853 | 0.373803971 |
| ENSG00000211445 | GPX3 | 8.273496575 | 0.822282123 | 0.590973142 |
| ENSG00000130208 | APOC1 | 1.540909104 | 0.735682339 | 0.828478314 |
| ENSG00000154096 | THY1 | 3.259615413 | 0.685544085 | 1 |
| ENSG00000161714 | PLCD3 | 2.026888129 | 0.673915744 | 0.980843948 |
| ENSG00000064655 | EYA2 | 1.321625886 | 0.661452295 | 1 |
| ENSG00000102854 | MSLN | 21.495682 | 0.655281114 | 1 |
| ENSG00000266714 | MYO15B | 1.114195814 | 0.654226692 | 1 |
| ENSG00000121716 | PILRB | 1.700926588 | 0.636885094 | 1 |
| ENSG00000108465 | CDK5RAP3 | 3.188496531 | 0.630792012 | 1 |
| ENSG00000164050 | PLXNB1 | 4.092298987 | 0.626795556 | 1 |
| ENSG00000267368 | UPK3BL1 | 1.534982531 | 0.623249748 | 1 |
| ENSG00000172890 | NADSYN1 | 1.38089162 | 0.587982939 | 1 |
| ENSG00000012171 | SEMA3B | 1.638697567 | 0.585072821 | 1 |
| ENSG00000132635 | PCED1A | 1.309772739 | 0.576702944 | 1 |
| ENSG00000127586 | CHTF18 | 1.443120642 | 0.575447211 | 1 |
| ENSG00000181404 | WASHC1 | 2.089117151 | 0.57377765 | 1 |
| ENSG00000198804 | MT-CO1 | 113.8642929 | 0.556594127 | 1 |
| ENSG00000113946 | CLDN16 | 3.899685349 | 0.556370157 | 1 |
| ENSG00000198888 | MT-ND1 | 92.01301653 | 0.543330658 | 1 |

| | | Cluster 7 | Cluster 7 Log2 | Cluster 7 |
|---|---|---|---|---|
| FeatureID | FeatureName | Average | Fold Change | P-Value |
| ENSG00000269821 | KCNQ1OT1 | 4.912998915 | 2.041638562 | 8.59E−08 |
| ENSG00000285437 | POLR2J3 | 1.557532392 | 2.012532771 | 1.62E−06 |
| ENSG00000255823 | MTRNR2L8 | 4.364476638 | 1.801689671 | 8.02E−06 |
| ENSG00000269028 | MTRNR2L12 | 14.19386041 | 1.657116714 | 0.000117994 |
| ENSG00000230590 | FTX | 1.181693054 | 1.366589619 | 0.006503258 |
| ENSG00000244879 | GABPB1-AS1 | 1.113974254 | 1.345431984 | 0.008460782 |
| ENSG00000135968 | GCC2 | 1.046255455 | 1.309470187 | 0.014728657 |
| ENSG00000228253 | MT-ATP8 | 2.790014546 | 1.274518325 | 0.014728657 |
| ENSG00000117724 | CENPF | 2.126370309 | 1.263689316 | 0.014857233 |
| ENSG00000144674 | GOLGA4 | 2.329526708 | 1.220111572 | 0.022099397 |
| ENSG00000198763 | MT-ND2 | 109.1220738 | 1.189348254 | 0.03099953 |
| ENSG00000171634 | BPTF | 1.845337291 | 1.182953288 | 0.033118606 |
| ENSG00000168137 | SETD5 | 1.395007273 | 1.150502131 | 0.045913317 |
| ENSG00000198804 | MT-CO1 | 161.1436557 | 1.123212493 | 0.053772173 |
| ENSG00000118058 | KMT2A | 1.642180892 | 1.116634443 | 0.068575354 |
| ENSG00000244754 | N4BP2L2 | 1.97400301 | 1.092091539 | 0.064364926 |
| ENSG00000112739 | PRPF4B | 2.390473628 | 1.08757192 | 0.064364926 |
| ENSG00000174501 | ANKRD36C | 0.643328597 | 1.082166959 | 0.123692251 |
| ENSG00000162599 | NFIA | 2.07219527 | 1.068520203 | 0.071895646 |
| ENSG00000133226 | SRRM1 | 1.350990053 | 1.055856537 | 0.085398589 |

| | | Cluster 8 | Cluster 8 Log2 | Cluster 8 |
|---|---|---|---|---|
| FeatureID | FeatureName | Average | Fold Change | P-Value |
| ENSG00000198899 | MT-ATP6 | 70.44286039 | 0.769837269 | 1 |
| ENSG00000198763 | MT-ND2 | 86.64966255 | 0.769278304 | 1 |
| ENSG00000198938 | MT-CO3 | 73.69046622 | 0.70936264 | 1 |
| ENSG00000198886 | MT-ND4 | 125.9175888 | 0.663928841 | 1 |
| ENSG00000198727 | MT-CYB | 46.9601721 | 0.640994288 | 1 |
| ENSG00000198804 | MT-CO1 | 122.2432143 | 0.637120149 | 1 |
| ENSG00000198888 | MT-ND1 | 98.85420689 | 0.625654489 | 1 |
| ENSG00000198786 | MT-ND5 | 11.00751014 | 0.579040721 | 1 |
| ENSG00000198712 | MT-CO2 | 148.6872611 | 0.563569105 | 1 |
| ENSG00000128422 | KRT17 | 0.843128436 | 0.550128107 | 1 |
| ENSG00000198840 | MT-ND3 | 78.0518343 | 0.524650385 | 1 |
| ENSG00000269028 | MTRNR2L12 | 7.31231761 | 0.508814006 | 1 |
| ENSG00000005884 | ITGA3 | 1.363578088 | 0.464408053 | 1 |
| ENSG00000105357 | MYH14 | 1.217852186 | 0.456564185 | 1 |
| ENSG00000119707 | RBM25 | 2.128639077 | 0.45620934 | 1 |
| ENSG00000225663 | MCRIP1 | 1.576962445 | 0.450005903 | 1 |
| ENSG00000136235 | GPNMB | 0.811901457 | 0.433631342 | 1 |
| ENSG00000228253 | MT-ATP8 | 1.691461369 | 0.428871042 | 1 |
| ENSG00000025708 | TYMP | 0.681789044 | 0.42772351 | 1 |
| ENSG00000175567 | UCP2 | 1.946481698 | 0.427372689 | 1 |

| | | Cluster 9 | Cluster 9 Log2 | Cluster 9 |
|---|---|---|---|---|
| FeatureID | FeatureName | Average | Fold Change | P-Value |
| ENSG00000169715 | MT1E | 2.331217707 | 1.686555808 | 7.48E−06 |
| ENSG00000113657 | DPYSL3 | 3.728034883 | 1.555793862 | 0.000115888 |
| ENSG00000204287 | HLA-DRA | 7.60914562 | 1.35648771 | 0.002784973 |
| ENSG00000143153 | ATP1B1 | 1.591351075 | 1.354101653 | 0.002700035 |
| ENSG00000179344 | HLA-DQB1 | 1.482922344 | 1.353479535 | 0.003720681 |
| ENSG00000231389 | HLA-DPA1 | 2.238734378 | 1.351290136 | 0.003099599 |
| ENSG00000176171 | BNIP3 | 1.020505699 | 1.321655645 | 0.00609679 |
| ENSG00000019582 | CD74 | 15.91669983 | 1.287949513 | 0.006225755 |
| ENSG00000198502 | HLA-DRB5 | 1.046018342 | 1.234778038 | 0.019994873 |
| ENSG00000196126 | HLA-DRB1 | 3.39318145 | 1.191597028 | 0.026217647 |
| ENSG00000223865 | HLA-DPB1 | 1.444653381 | 1.174476731 | 0.030512352 |
| ENSG00000175265 | GOLGA8A | 2.682016541 | 1.166405308 | 0.024606648 |
| ENSG00000187837 | HIST1H1C | 1.457409702 | 1.13498075 | 0.041149498 |
| ENSG00000137673 | MMP7 | 0.889753407 | 1.071051913 | 0.20101047 |
| ENSG00000158406 | HIST1H4H | 1.074720065 | 1.063326409 | 0.090647005 |
| ENSG00000134419 | RPS15A | 14.51669357 | 1.040696112 | 0.096360729 |
| ENSG00000113719 | ERGIC1 | 1.4414643 | 1.031609626 | 0.101946736 |
| ENSG00000204628 | RACK1 | 14.23286542 | 0.968248201 | 0.175234943 |
| ENSG00000171314 | PGAM1 | 2.15900737 | 0.967371541 | 0.160414145 |
| ENSG00000162366 | PDZK1IP1 | 2.796823432 | 0.951731733 | 0.175234943 |

TABLE 6
Top 20 Down-regulated genes for each cluster (1-9)

| | | Cluster 1 | Cluster 1 Log2 | Cluster 1 |
|---|---|---|---|---|
| FeatureID | FeatureName | Average | Fold Change | P-Value |
| ENSG00000211679 | IGLC3 | 0.034422934 | −4.34767347 | 6.20E−24 |
| ENSG00000211899 | IGHM | 0.014885593 | −4.244674937 | 2.38E−11 |
| ENSG00000132465 | JCHAIN | 0.045587129 | −4.086712653 | 8.58E−27 |
| ENSG00000211685 | IGLC7 | 0.007442797 | −4.075607407 | 0.00870123 |
| ENSG00000211893 | IGHG2 | 0.011164195 | −4.05016239 | 5.62E−15 |
| ENSG00000211677 | IGLC2 | 0.142343485 | −3.968462894 | 1.56E−23 |
| ENSG00000211675 | IGLC1 | 0.182348517 | −3.938103883 | 1.83E−24 |
| ENSG00000211897 | IGHG3 | 0.534951007 | −3.931388261 | 7.76E−29 |
| ENSG00000211892 | IGHG4 | 2.453331833 | −3.814634793 | 2.84E−29 |
| ENSG00000211896 | IGHG1 | 0.237239142 | −3.673998329 | 1.55E−26 |
| ENSG00000211592 | IGKC | 3.520442797 | −3.578329904 | 2.63E−27 |
| ENSG00000211890 | IGHA2 | 0.02139804 | −3.494737661 | 5.59E−09 |
| ENSG00000170476 | MZB1 | 0.020467691 | −3.474156783 | 2.68E−16 |
| ENSG00000211895 | IGHA1 | 0.502388771 | −3.224394735 | 7.50E−19 |
| ENSG00000112936 | C7 | 0.018606992 | −2.968692203 | 3.85E−11 |
| ENSG00000137077 | CCL21 | 0.024189089 | −2.681467248 | 6.58E−07 |
| ENSG00000111341 | MGP | 0.099547405 | −2.532829353 | 7.20E−14 |
| ENSG00000107562 | CXCL12 | 0.048378178 | −2.194317023 | 1.46E−09 |
| ENSG00000214548 | MEG3 | 0.123736494 | −2.160188661 | 4.36E−12 |
| ENSG00000115380 | EFEMP1 | 0.049308528 | −2.139737744 | 1.25E−08 |

| | | Cluster 2 | Cluster 2 Log2 | Cluster 2 |
|---|---|---|---|---|
| FeatureID | FeatureName | Average | Fold Change | P-Value |
| ENSG00000211685 | IGLC7 | 0.026157744 | −1.842238329 | 1 |
| ENSG00000142973 | CYP4B1 | 0.496997143 | −0.777580643 | 1 |
| ENSG00000131174 | COX7B | 1.177098495 | −0.692993971 | 1 |
| ENSG00000185686 | PRAME | 0.871924811 | −0.688908114 | 1 |
| ENSG00000116171 | SCP2 | 0.627785864 | −0.660402245 | 1 |
| ENSG00000172115 | CYCS | 1.20325624 | −0.647209605 | 1 |
| ENSG00000126749 | EMG1 | 0.845767067 | −0.616918264 | 1 |
| ENSG00000150687 | PRSS23 | 0.658303233 | −0.6046386 | 1 |
| ENSG00000241685 | ARPC1A | 0.954757669 | −0.594100723 | 1 |
| ENSG00000112308 | C6orf62 | 1.582543533 | −0.579649991 | 1 |
| ENSG00000143977 | SNRPG | 1.068107894 | −0.573037339 | 1 |
| ENSG00000132646 | PCNA | 0.605987744 | −0.567557482 | 1 |
| ENSG00000265681 | RPL17 | 9.368832098 | −0.561347902 | 1 |
| ENSG00000134644 | PUM1 | 0.771653458 | −0.557173356 | 1 |
| ENSG00000131981 | LGALS3 | 0.749855338 | −0.556335772 | 1 |
| ENSG00000138029 | HADHB | 0.723697593 | −0.552963221 | 1 |
| ENSG00000124939 | SCGB2A1 | 1.909515337 | −0.551031585 | 1 |
| ENSG00000006625 | GGCT | 0.588549248 | −0.542973255 | 1 |
| ENSG00000172586 | CHCHD1 | 0.802170827 | −0.537886185 | 1 |
| ENSG00000112118 | MCM3 | 0.675741729 | −0.53290724 | 1 |

| | | Cluster 3 | Cluster 3 Log2 | Cluster 3 |
|---|---|---|---|---|
| FeatureID | FeatureName | Average | Fold Change | P-Value |
| ENSG00000142973 | CYP4B1 | 0.429157313 | −1.038420424 | 0.121760124 |
| ENSG00000145390 | USP53 | 0.464527421 | −1.017932018 | 0.138387688 |
| ENSG00000171517 | LPAR3 | 0.561205716 | −0.954846843 | 0.187559563 |
| ENSG00000124939 | SCGB2A1 | 1.681259142 | −0.772673327 | 0.452172035 |
| ENSG00000113946 | CLDN16 | 1.709555228 | −0.755444319 | 0.479363692 |
| ENSG00000242265 | PEG10 | 0.775784373 | −0.747377091 | 0.504739077 |
| ENSG00000131747 | TOP2A | 0.639019954 | −0.714038563 | 0.59466643 |
| ENSG00000111371 | SLC38A1 | 1.016301108 | −0.708448915 | 0.585712939 |
| ENSG00000204370 | SDHD | 1.072893281 | −0.6991241 | 0.610706656 |
| ENSG00000163558 | PRKCI | 1.756715373 | −0.694076138 | 0.611296909 |
| ENSG00000134258 | VTCN1 | 0.570637745 | −0.678012908 | 0.686277041 |
| ENSG00000169021 | UQCRFS1 | 0.782858394 | −0.674520012 | 0.667931309 |
| ENSG00000072274 | TFRC | 1.348780125 | −0.671531553 | 0.661824789 |
| ENSG00000205981 | DNAJC19 | 0.584785789 | −0.657797332 | 0.707784155 |
| ENSG00000123472 | ATPAF1 | 0.646093976 | −0.617713904 | 0.819369311 |
| ENSG00000180530 | NRIP1 | 0.976214986 | −0.611935395 | 1 |
| ENSG00000172115 | CYCS | 1.254459837 | −0.611024991 | 0.810199282 |
| ENSG00000163331 | DAPL1 | 1.131843462 | −0.61081892 | 0.823834562 |
| ENSG00000154640 | BTG3 | 0.712118178 | −0.6106245 | 0.831050686 |
| ENSG00000211899 | IGHM | 0.148554454 | −0.608742768 | 1 |
| | | Cluster 4 | Cluster 4 Log2 | Cluster 4 |
| FeatureID | FeatureName | Average | Fold Change | P-Value |
| ENSG00000211685 | IGLC7 | 0.001254216 | −5.704331421 | 0.003090151 |
| ENSG00000211677 | IGLC2 | 0.048914412 | −5.372750153 | 4.94E−26 |
| ENSG00000112936 | C7 | 0.002508431 | −5.268863913 | 2.08E−15 |
| ENSG00000211893 | IGHG2 | 0.003762647 | −5.207042328 | 2.50E−14 |
| ENSG00000211897 | IGHG3 | 0.219487746 | −5.100347965 | 2.30E−31 |
| ENSG00000211675 | IGLC1 | 0.080269804 | −4.995274285 | 3.00E−25 |
| ENSG00000211679 | IGLC3 | 0.021321667 | −4.873416451 | 3.89E−20 |
| ENSG00000211892 | IGHG4 | 1.160149515 | −4.783790465 | 2.30E−31 |
| ENSG00000211899 | IGHM | 0.00877951 | −4.781014423 | 7.85E−10 |
| ENSG00000211592 | IGKC | 1.505058831 | −4.699162483 | 2.30E−31 |
| ENSG00000132465 | JCHAIN | 0.028846961 | −4.595946797 | 6.15E−23 |
| ENSG00000211895 | IGHA1 | 0.181861275 | −4.590128669 | 3.99E−24 |
| ENSG00000211896 | IGHG1 | 0.125421569 | −4.475295617 | 4.00E−26 |
| ENSG00000170476 | MZB1 | 0.012542157 | −3.997531538 | 1.75E−14 |
| ENSG00000091986 | CCDC80 | 0.08152402 | −3.710501651 | 1.15E−23 |
| ENSG00000122641 | INHBA | 0.062710785 | −3.537771798 | 5.75E−21 |
| ENSG00000111341 | MGP | 0.046405981 | −3.533935305 | 1.86E−17 |
| ENSG00000144810 | COL8A1 | 0.066473432 | −3.443284472 | 4.00E−20 |
| ENSG00000103196 | CRISPLD2 | 0.043897549 | −3.389545395 | 6.81E−18 |
| ENSG00000106366 | SERPINE1 | 0.035118039 | −3.384007212 | 9.89E−17 |
| | | Cluster 5 | Cluster 5 Log2 | Cluster 5 |
| FeatureID | FeatureName | Average | Fold Change | P-Value |
| ENSG00000185686 | PRAME | 0.358985671 | −1.987436903 | 0.006893748 |
| ENSG00000142973 | CYP4B1 | 0.253130922 | −1.760093302 | 0.027404386 |
| ENSG00000113946 | CLDN16 | 0.975704643 | −1.551562585 | 0.053027061 |
| ENSG00000144749 | LRIG1 | 0.82382609 | −1.464349139 | 0.078483728 |
| ENSG00000124939 | SCGB2A1 | 1.040137969 | −1.449643109 | 0.085246767 |
| ENSG00000172005 | MAL | 0.386599953 | −1.409342372 | 0.110409565 |
| ENSG00000272398 | CD24 | 2.448466368 | −1.395389394 | 0.099082553 |
| ENSG00000164825 | DEFB1 | 0.998716545 | −1.384679295 | 0.11186879 |
| ENSG00000205981 | DNAJC19 | 0.34978091 | −1.38126045 | 0.124592353 |
| ENSG00000247516 | MIR4458HG | 0.409611855 | −1.378463923 | 0.124063315 |
| ENSG00000270170 | NCBP2AS2 | 0.391202333 | −1.35936364 | 0.133469233 |
| ENSG00000104413 | ESRP1 | 0.575297549 | −1.344602767 | 0.13262815 |
| ENSG00000205213 | LGR4 | 0.409611855 | −1.301154829 | 0.163965853 |
| ENSG00000147676 | MAL2 | 0.81922371 | −1.282802437 | 0.161790594 |
| ENSG00000184292 | TACSTD2 | 0.759392765 | −1.274719957 | 0.172436844 |
| ENSG00000119705 | SLIRP | 0.787007047 | −1.262537254 | 0.175151105 |
| ENSG00000161179 | YDJC | 0.414214235 | −1.254674773 | 0.194403257 |
| ENSG00000172115 | CYCS | 0.800814188 | −1.248377452 | 0.185226003 |
| ENSG00000260260 | SNHG19 | 1.942204525 | −1.241429079 | 0.18184398 |
| ENSG00000064655 | EYA2 | 0.381997573 | −1.236323722 | 0.211169349 |
| | | Cluster 6 | Cluster 6 Log2 | Cluster 6 |
| FeatureID | FeatureName | Average | Fold Change | P-Value |
| ENSG00000211685 | IGLC7 | 0 | −5.269508748 | 1 |
| ENSG00000211899 | IGHM | 0.00888986 | −4.349389525 | 0.021710528 |
| ENSG00000211677 | IGLC2 | 0.103715036 | −4.087255819 | 8.68E−06 |
| ENSG00000211592 | IGKC | 2.038741276 | −4.068538147 | 4.42E−08 |
| ENSG00000211897 | IGHG3 | 0.465236018 | −3.818713231 | 7.94E−07 |
| ENSG00000211892 | IGHG4 | 2.041704563 | −3.773257951 | 5.21E−07 |
| ENSG00000132465 | JCHAIN | 0.047412588 | −3.65954371 | 1.46E−05 |
| ENSG00000211896 | IGHG1 | 0.195576925 | −3.634658556 | 2.07E−06 |
| ENSG00000211679 | IGLC3 | 0.044449301 | −3.606357732 | 0.00021597 |
| ENSG00000211675 | IGLC1 | 0.183723778 | −3.602324136 | 4.27E−05 |
| ENSG00000211890 | IGHA2 | 0.014816434 | −3.526180652 | 0.074394605 |
| ENSG00000211895 | IGHA1 | 0.411896857 | −3.212908895 | 0.0001842 |
| ENSG00000211893 | IGHG2 | 0.014816434 | −3.178619607 | 0.020577948 |
| ENSG00000112936 | C7 | 0.011853147 | −3.087030696 | 0.01134634 |
| ENSG00000106483 | SFRP4 | 0.038522728 | −3.067211544 | 0.000184253 |
| ENSG00000170476 | MZB1 | 0.020743007 | −3.024925194 | 0.003518548 |
| ENSG00000144810 | COL8A1 | 0.082972029 | −2.917594709 | 3.56E−05 |
| ENSG00000145423 | SFRP2 | 0.109641609 | −2.765315876 | 0.000115648 |
| ENSG00000115380 | EFEMP1 | 0.026669581 | −2.666579701 | 0.006509245 |
| ENSG00000111341 | MGP | 0.077045455 | −2.59787675 | 0.00134628 |
| | | Cluster 7 | Cluster 7 Log2 | Cluster 7 |
| FeatureID | FeatureName | Average | Fold Change | P-Value |
| ENSG00000211685 | IGLC7 | 0.00677188 | −3.468593919 | 1 |
| ENSG00000211892 | IGHG4 | 3.856585641 | −2.833514736 | 0.000368487 |
| ENSG00000211592 | IGKC | 5.177102234 | −2.698734181 | 0.000611466 |
| ENSG00000211675 | IGLC1 | 0.369067458 | −2.580965874 | 0.018767105 |
| ENSG00000211899 | IGHM | 0.03724534 | −2.542056081 | 0.450604052 |
| ENSG00000211896 | IGHG1 | 0.440172198 | −2.447899677 | 0.009283128 |
| ENSG00000211897 | IGHG3 | 1.225710274 | −2.397154264 | 0.014728657 |
| ENSG00000211677 | IGLC2 | 0.355523698 | −2.304623826 | 0.064364926 |
| ENSG00000211895 | IGHA1 | 0.771994316 | −2.284720597 | 0.043449691 |
| ENSG00000132465 | JCHAIN | 0.132051659 | −2.201462476 | 0.043449691 |
| ENSG00000211890 | IGHA2 | 0.04063128 | −2.187894319 | 0.613213167 |
| ENSG00000211679 | IGLC3 | 0.121893839 | −2.17319094 | 0.106006903 |
| ENSG00000137077 | CCL21 | 0.02708752 | −2.109096673 | 0.447745512 |
| ENSG00000170476 | MZB1 | 0.04063128 | −2.105077034 | 0.11488656 |
| ENSG00000107562 | CXCL12 | 0.04063128 | −2.096527705 | 0.064364926 |
| ENSG00000139329 | LUM | 0.382611218 | −2.033905188 | 0.013040877 |
| ENSG00000111341 | MGP | 0.115121959 | −2.007931332 | 0.056295035 |
| ENSG00000105664 | COMP | 0.09142038 | −1.973444662 | 0.044640001 |
| ENSG00000211893 | IGHG2 | 0.03724534 | −1.95453334 | 0.371215671 |
| ENSG00000137673 | MMP7 | 0.125279779 | −1.931010247 | 0.070535564 |

| | | Cluster 8 | Cluster 8 Log2 | Cluster 8 |
|---|---|---|---|---|
| FeatureID | FeatureName | Average | Fold Change | P-Value |
| ENSG00000170476 | MZB1 | 0.036431476 | −2.157623658 | 1 |
| ENSG00000211685 | IGLC7 | 0.020817986 | −2.065923824 | 1 |
| ENSG00000211679 | IGLC3 | 0.145725903 | −1.871856671 | 1 |
| ENSG00000211677 | IGLC2 | 0.499631666 | −1.7761693 | 1 |
| ENSG00000211897 | IGHG3 | 1.837187271 | −1.775426315 | 1 |
| ENSG00000211899 | IGHM | 0.062453958 | −1.766363542 | 1 |
| ENSG00000211893 | IGHG2 | 0.041635972 | −1.71872897 | 1 |
| ENSG00000211675 | IGLC1 | 0.676584548 | −1.666585805 | 1 |
| ENSG00000132465 | JCHAIN | 0.192566371 | −1.618249117 | 1 |
| ENSG00000211592 | IGKC | 11.00751014 | −1.565368861 | 1 |
| ENSG00000211892 | IGHG4 | 9.003778979 | −1.563739464 | 1 |
| ENSG00000211890 | IGHA2 | 0.067658455 | −1.420456584 | 1 |
| ENSG00000211895 | IGHA1 | 1.400009564 | −1.384213881 | 1 |
| ENSG00000211896 | IGHG1 | 1.020081318 | −1.189714367 | 1 |
| ENSG00000164932 | CTHRC1 | 0.208179861 | −1.117762755 | 1 |
| ENSG00000143248 | RGS5 | 0.088476441 | −1.089770566 | 1 |
| ENSG00000112936 | C7 | 0.052044965 | −1.06264124 | 1 |
| ENSG00000137077 | CCL21 | 0.062453958 | −0.907742155 | 1 |
| ENSG00000166147 | FBN1 | 0.140521406 | −0.873278746 | 1 |
| ENSG00000011465 | DCN | 0.686993541 | −0.860828226 | 1 |

| | | Cluster 9 | Cluster 9 Log2 | Cluster 9 |
|---|---|---|---|---|
| FeatureID | FeatureName | Average | Fold Change | P-Value |
| ENSG00000211899 | IGHM | 0.019134482 | −3.42164908 | 0.127912814 |
| ENSG00000170476 | MZB1 | 0.019134482 | −3.104375526 | 0.003429305 |
| ENSG00000211893 | IGHG2 | 0.015945402 | −3.063311021 | 0.030371126 |
| ENSG00000211895 | IGHA1 | 0.471983886 | −3.006759484 | 0.000666915 |
| ENSG00000211592 | IGKC | 4.337149222 | −2.963109052 | 2.26E−05 |
| ENSG00000211679 | IGLC3 | 0.076537927 | −2.840817177 | 0.007544236 |
| ENSG00000211677 | IGLC2 | 0.251937345 | −2.810005384 | 0.005209487 |
| ENSG00000211892 | IGHG4 | 4.043753833 | −2.770447151 | 0.000125981 |
| ENSG00000211675 | IGLC1 | 0.338042513 | −2.714885702 | 0.005209487 |
| ENSG00000211890 | IGHA2 | 0.028701723 | −2.665913681 | 0.356172087 |
| ENSG00000211897 | IGHG3 | 1.02688386 | −2.661564655 | 0.001422709 |
| ENSG00000112936 | C7 | 0.019134482 | −2.479796646 | 0.072831406 |
| ENSG00000132465 | JCHAIN | 0.114806891 | −2.409363309 | 0.010211026 |
| ENSG00000211896 | IGHG1 | 0.484740207 | −2.313745681 | 0.008131295 |
| ENSG00000211685 | IGLC7 | 0.025512642 | −1.955420299 | 1 |
| ENSG00000111341 | MGP | 0.143508614 | −1.697792982 | 0.152652746 |
| ENSG00000142973 | CYP4B1 | 0.277449987 | −1.659066516 | 0.045435812 |
| ENSG00000164825 | DEFB1 | 0.959913173 | −1.467341897 | 0.096360729 |
| ENSG00000137077 | CCL21 | 0.044647124 | −1.447079079 | 1 |
| ENSG00000134258 | VTCN1 | 0.341231593 | −1.428476861 | 0.150324187 |

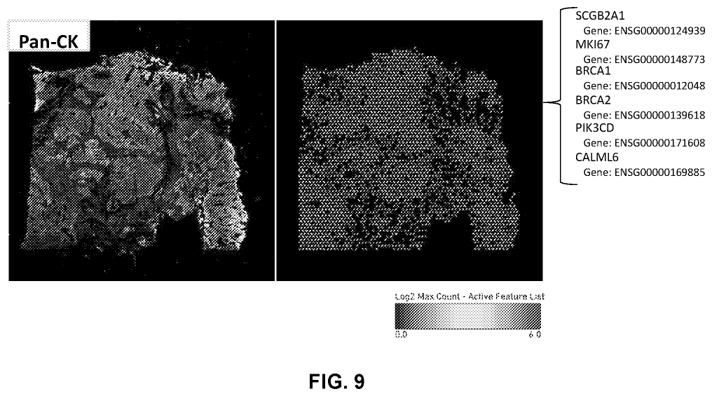

As shown in FIG. 9, Pan-CK staining (left panel) correlated with expression of cancer cell markers SCGB2A1, MKi67, BRCA1, BRCA2, PIK3CD, and CALML6 (right panel) as determined by spatial sequencing.

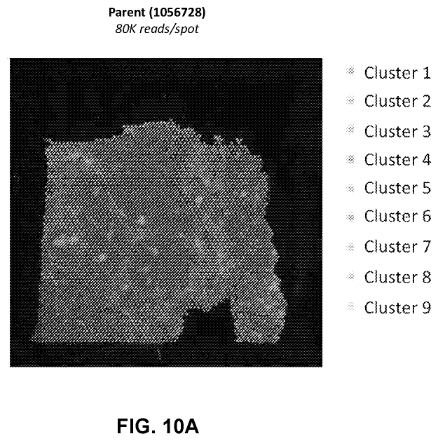

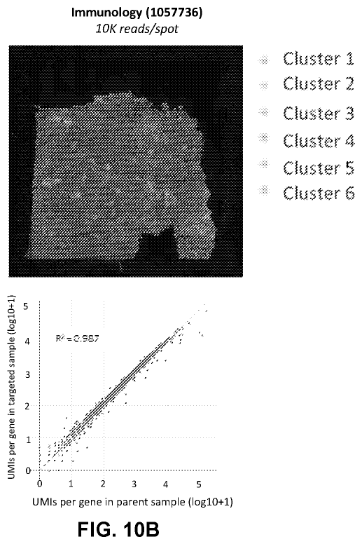

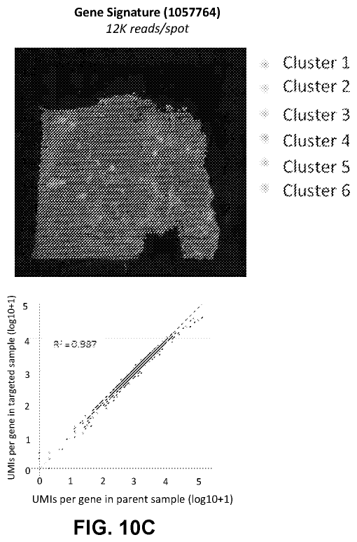

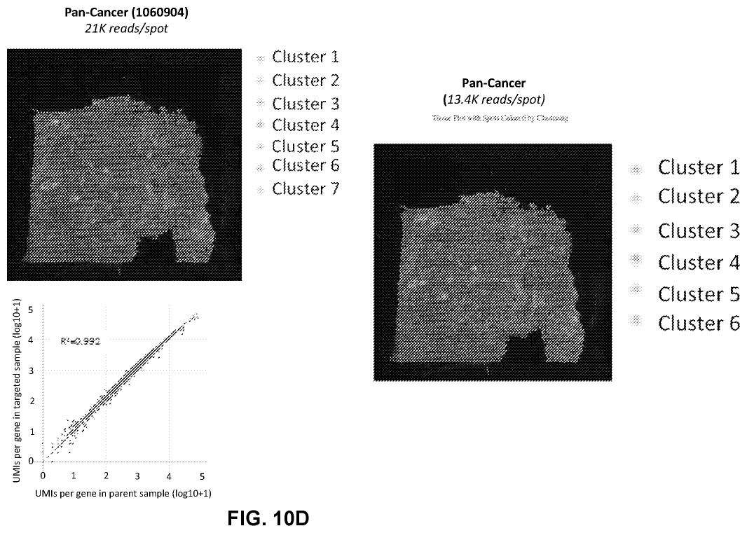

Quality and depth of gene expression profiling using targeted panels was assessed as shown in FIG. 10A-10D. FIG. 10A shows spot clusters of the Visium whole transcriptome gene expression library. FIG. 10B (top panel) shows spot clusters of the human immunology panel targeted library. The bottom panel of FIG. 10B shows Pearson correlation of log₁₀ UMI counts per gene between the parent whole transcriptome analysis (WTA) and the immunology targeted analysis (R²=0.987). FIG. 10C shows spot clusters of the human gene signature panel targeted library. The bottom panel of FIG. 10C shows the Pearson correlation of log₁₀ UMI counts per gene between parent whole transcriptome analysis (WTA) and the gene signature targeted analysis (R2=0.987). FIG. 10D shows spot clusters of the human pan-cancer panel targeted library (7 clusters, top left; or 6 clusters, top right). The bottom panel of FIG. 10D shows the Pearson correlation of log₁₀ UMI counts per gene between the parent whole transcriptome analysis (WTA) and the pan cancer targeted analysis (R²=0.992).

### Determination of Localization of Tumor Infiltrating Immune Cells

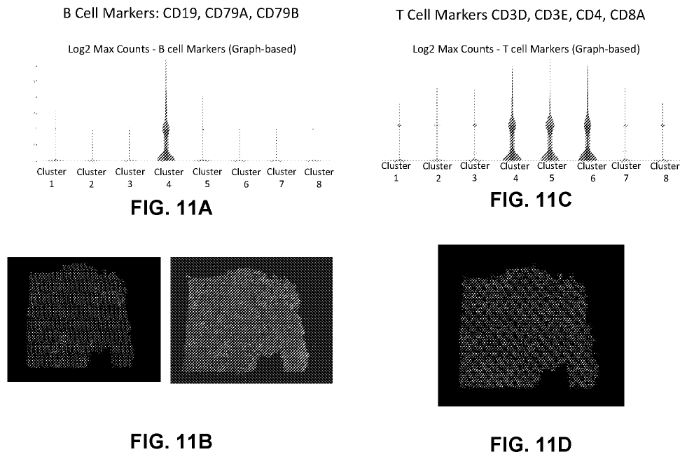

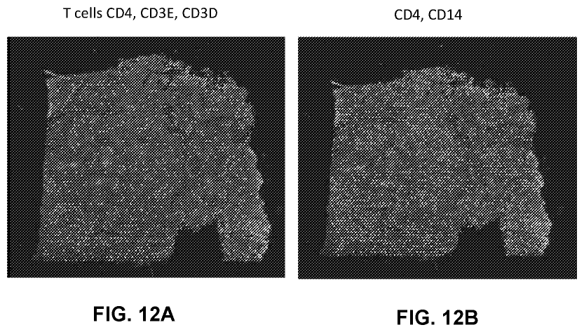

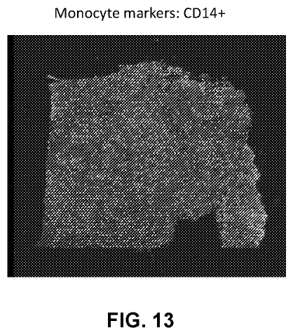

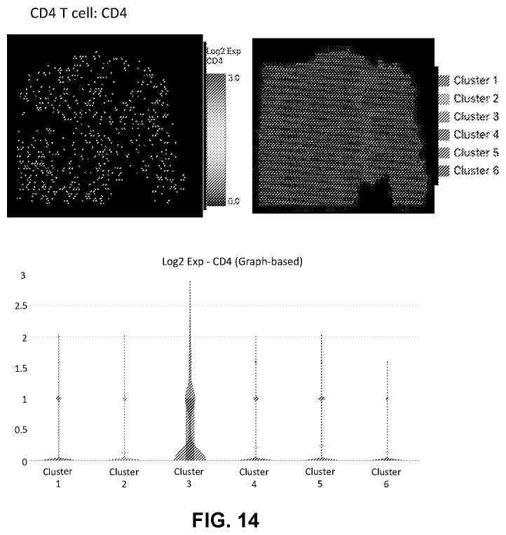

The methods described above were used to determine immune cell infiltration in a biological sample, by in part, identifying the abundance and/or location of a tumor infiltrating T-lymphocyte and a tumor infiltrating B cell (TIB) in a test biological sample. TIB were detected using gene expression of B cell markers CD19, CD79A and/or CD79B. Tumor infiltrating T-lymphocytes were detected using gene expression T cell markers CD3D, CD3E, CD4 and/or CD8A. Expression of B cell markers were seen in cluster 4 (FIG. 11A) and localized to specific areas within the CD45⁺ compartment (FIG. 11B) where T cell markers expression was seen in clusters 4, 5, and 6 (FIG. 11C) and were present throughout the tissue (FIG. 11D). Additional T cell markers overlaid with tissue sections stained with Pan-CK and CD45 showed presence of T cells throughout the ovarian tumor sections (FIGS. 12A-12B). As tumor infiltrating immune cells can also include tumor infiltrating monocytes, the spatial location of a monocyte marker CD14 was overlaid with tissue sections stained with Pan-CK and CD45 (FIG. 13). Looking at specific T cell markers showed gene expression for CD4 was restricted to cluster 3 (FIG. 14, lower panel) and was present throughout the sample (FIG. 14, upper panels), and gene expression for CD8A was not enriched in any of the clusters (FIG. 15, lower panel) and but was present throughout the sample (FIG. 15, upper panels).

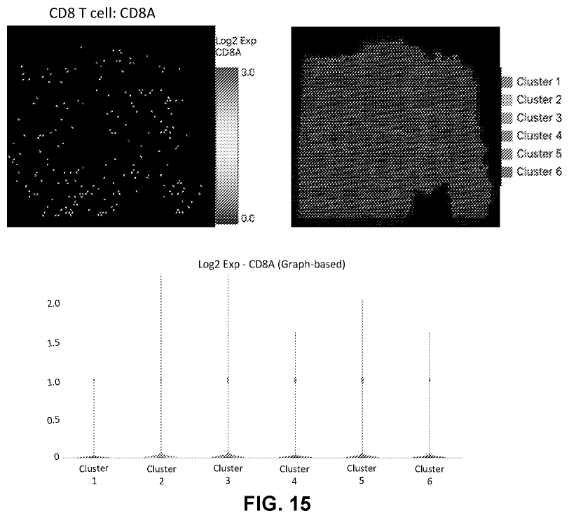

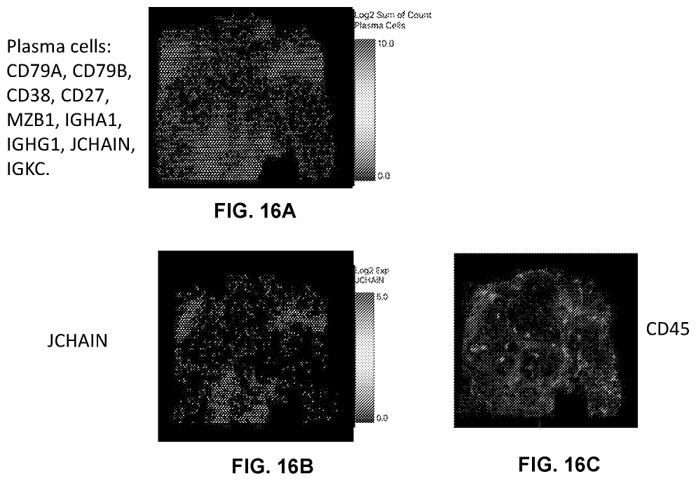

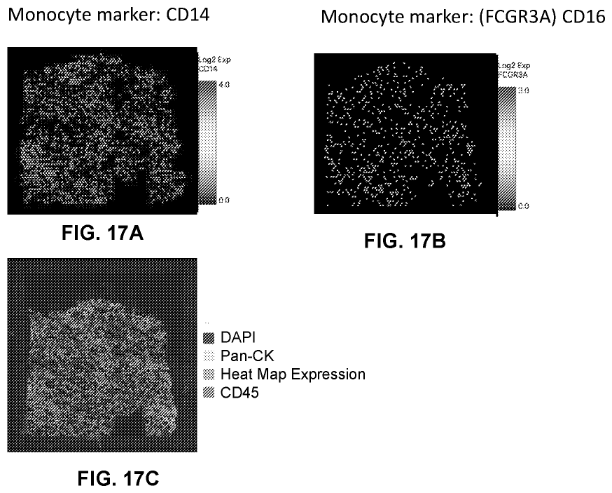

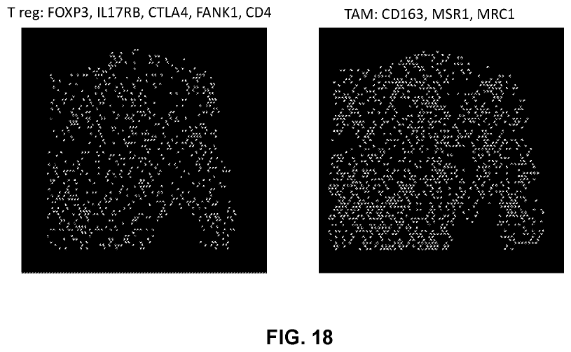

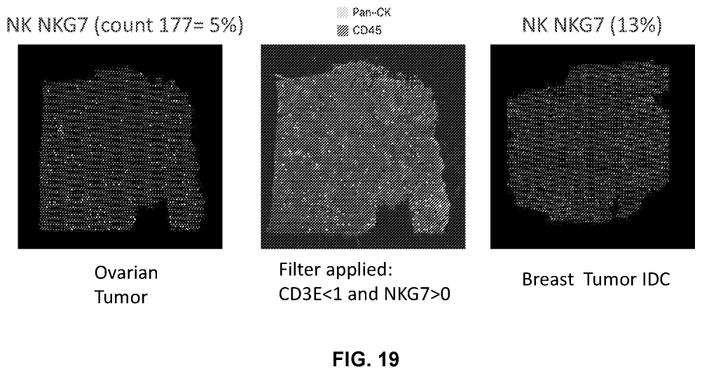

The methods described above were also used to determine presence and/or abundance of adaptive immune cells in the biological sample. Plasma B cells were shown to cluster in specific areas of the stromal compartment, suggesting a B-cell response against the tumor in the biological sample. FIG. 16A shows gene expression for plasma cell markers: CD79A, CD79B, CD38, CD27, MZB1, IGHA1, IGHG1, JCHAIN, and IGKC (top panel). FIG. 16B shows a gene expression heat map for JCHAIN (lower left panel), FIG. 16C shows CD45 expression in the same tissue section. Monocytes were detected using CD14 and CD16 (FCGR3A) (FIGS. 17A-B) and overlaid with the immunostain for DAPI, Pan-CK, and CD45 (FIG. 17C). T regulatory (Treg) cells were identified in the sample using FOXP3, IL17RB, CTLA4, FANK1, and CD4 (FIG. 18, left panel) and tumor associated macrophages (TAMs) were identified using CD163, MSR1, and MRC1 (FIG. 18, right panel). Natural killer (NK) cells were identified using NKG7 (FIG. 19, left panel) and merged with Pan-CK and CD45 staining as shown in FIG. 19, center panel. Abundance of NK cells in the ovarian tumor sample was 5% (177 NK barcodes counted) as compared to 13% in a breast invasive ductal carcinoma sample (FIG. 19, right panel))).

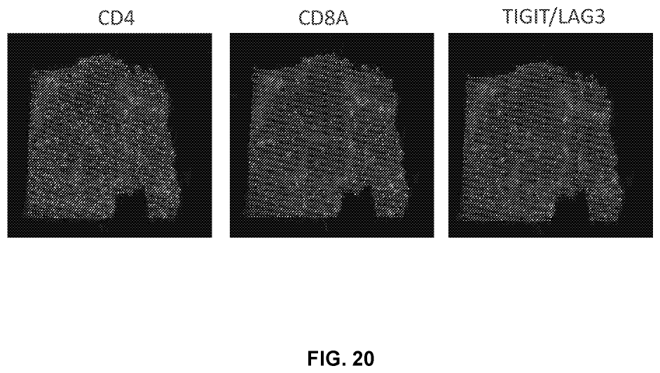

The diverse subsets of TILs present in the tumor sample was indicated by the presence of CD4, CD8A and TIGIT/Lag3 (FIG. 20). CD4, CD8A and TIGIT/Lag3 gene expression heat maps were merged with tissue sections stained with CD45 to show the diversity in both TIL type and TIL location (FIG. 20).

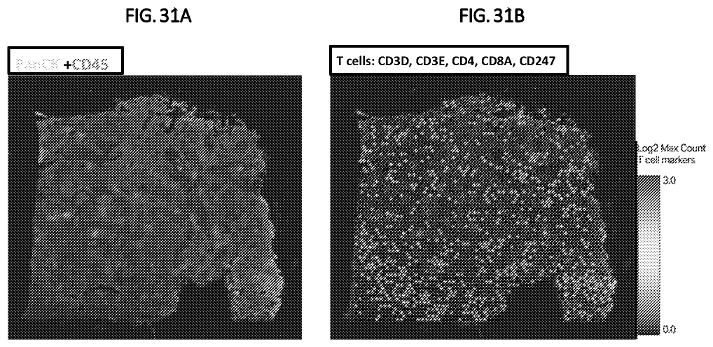

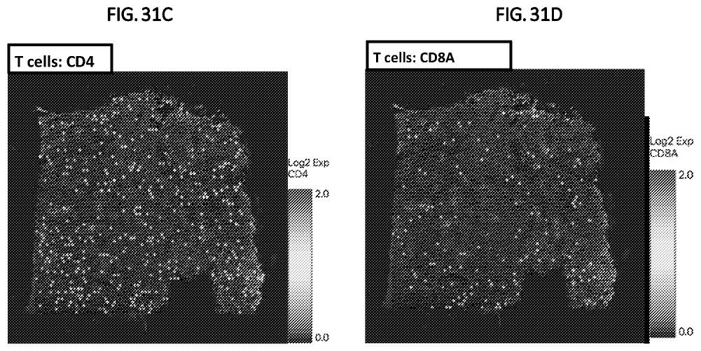

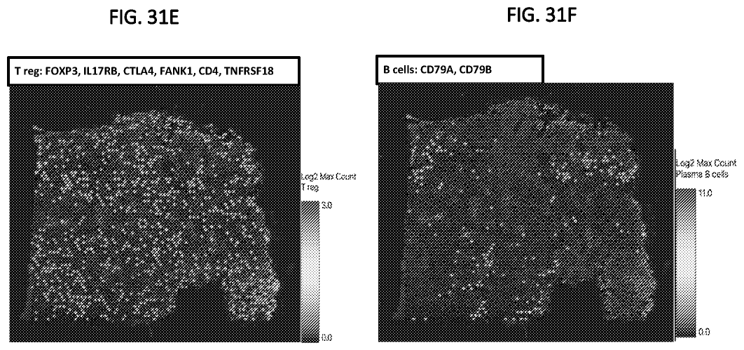

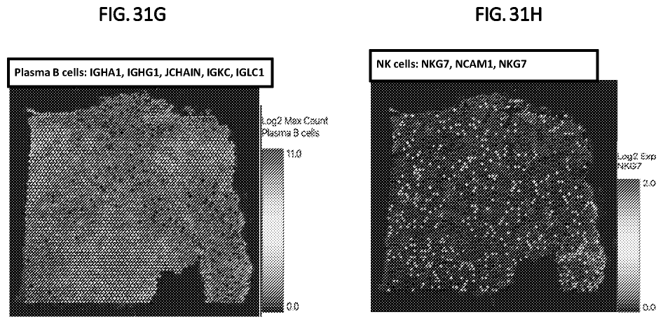

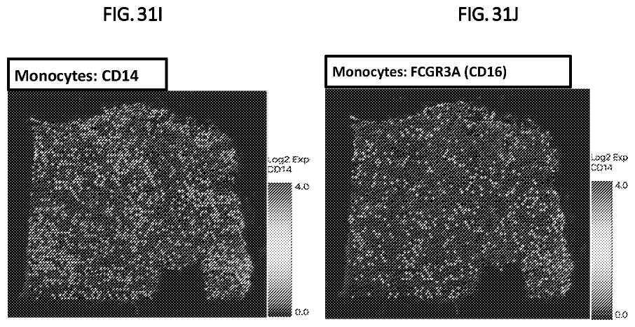

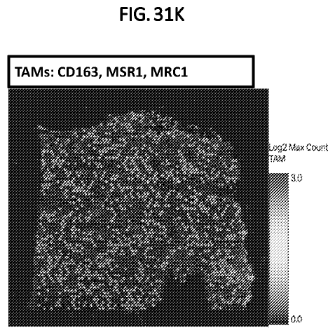

Immune cell expression co-localized with Pan-CK or CD45. Pan-CK or CD45 immunostaining is shown in FIG. 31A. As shown in FIGS. 31B-31K, the results herein show co-localized expression of Pan-CK and CD45 with expression of general T cell markers CD3D, CD3E, CD4, CD8A, and CD247 (FIG. 31B); helper T cell marker CD4 (FIG. 31C); cytotoxic T Cell marker CD8A (FIG. 31D); markers of Treg cells (FIG. 31E); markers of B cells (FIG. 31F); markers of plasma B cells (FIG. 31G); markers of NK cells (FIG. 31H), markers of CD14 monocytes (FIG. 31I); markers of CD16 monocytes (FIG. 31J); and markers of TAMs (FIG. 31K). FIG. 31B shows T cells dispersed throughout the Pan-CK and CD45 compartments, while FIGS. 31F and 31G show B cells localized to the stromal compartment. These images demonstrate the ability for one to determine immune cell infiltration overlapped with stromal and tumor compartments in a sample.

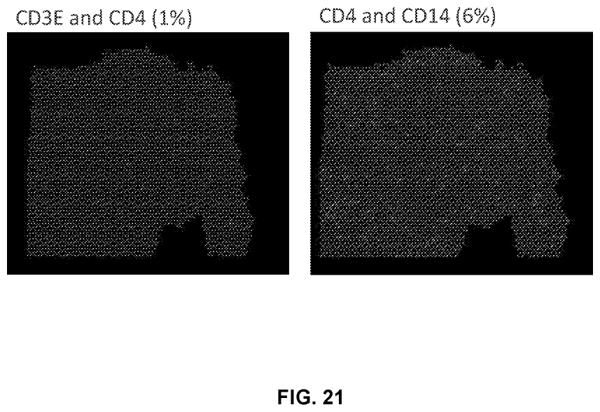

The methods described herein were also used to show that co-expression of immune cell markers can be used as a proxy for immune cell co-localization (FIG. 21).

### Determination of Localization of Stromal Cells

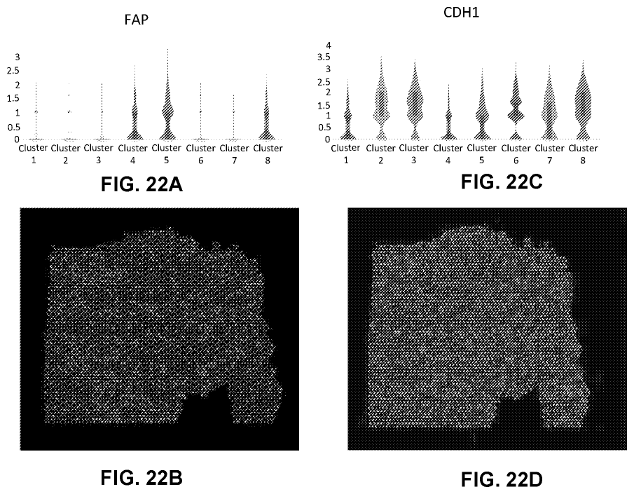

Using spatial gene expression of fibroblast activation protein (FAP) and cadherin-1 (CDH1), the abundance and/or location of stromal cells was identified in the ovarian cancer tissue section. FAP expression was seen in clusters 4, 5, and 8 (FIG. 22A) and showed some specific localization (FIG. 22B). CDH1 expression was seen in each of the clusters, likely due to its expression levels in the tissue section (FIG. 22C). FIG. 22D shows an overlay of CDH1 expression and CD45 immunostaining.

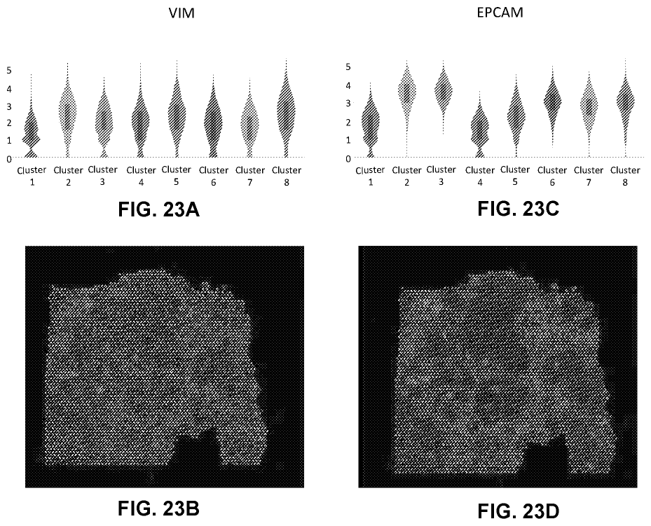

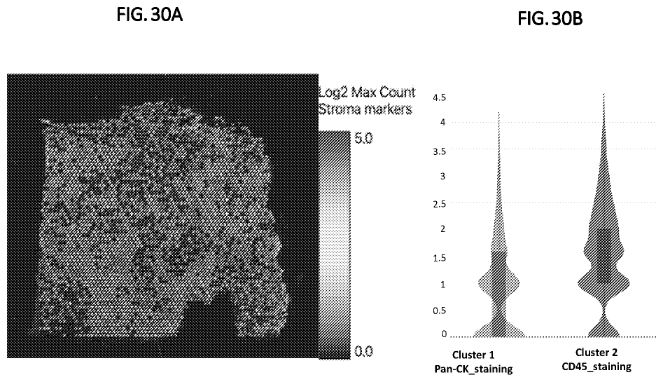

In addition, spatial gene expression for vimentin (VIM) and epithelial cell adhesion molecule (EPCAM) were also assessed to determine the abundance and/or spatial location of stromal cells. VIM expression was seen in each of the clusters, likely due to its expression levels in the tissue section (FIG. 23A), and localized throughout the tissue. FIG. 23B shows an overlay of VIM expression and CD45 immunostaining. EPCAM expression was seen in each of the clusters, likely due to its expression levels in the tissue section (FIG. 23C). FIG. 23D shows an overlay of EPCAM expression and CD45 immunostaining. Further, FIGS. 30A-30B show stromal-specific expression of FAP, VCAN, ACTA2, and PDGFRB in stromal compartments.

Expression profiling of the clusters revealed an abundance of B cell markers in cluster 4, T cell markers in clusters 4-6, and stromal markers FAP, CDH1, VIM, and EPCAM in each cluster, including clusters 4-6. These results indicate immune cell infiltration in the stromal compartment of the ovarian cancer tissue section.

### Determination of Localization of Cancer Genes

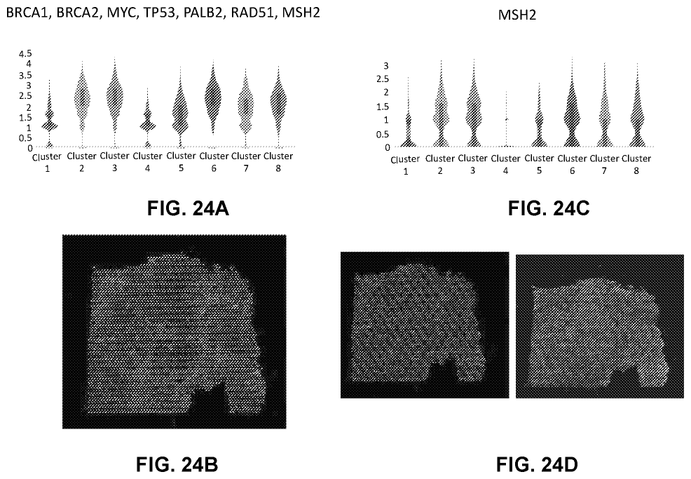

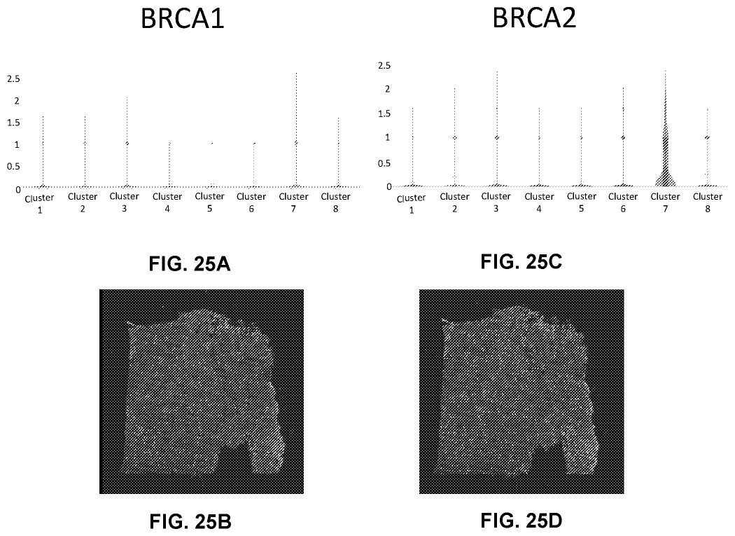

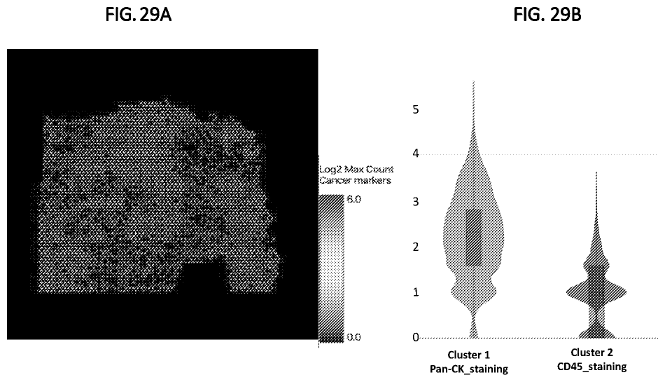

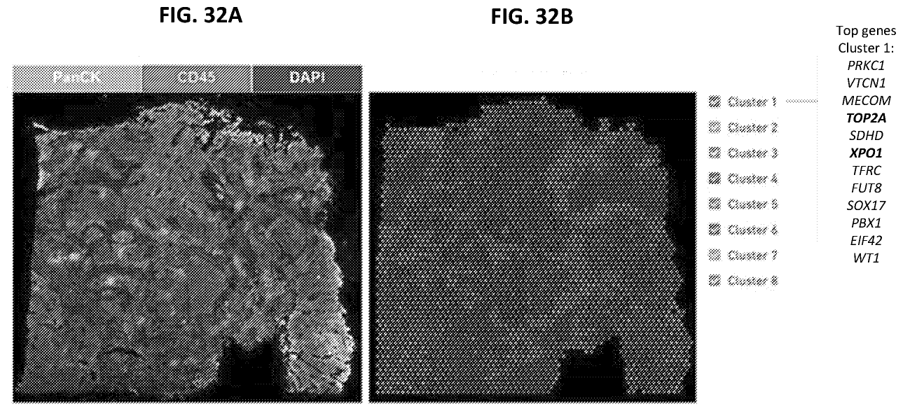

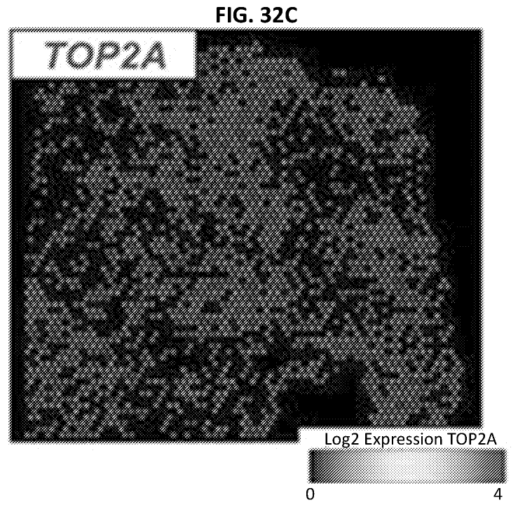

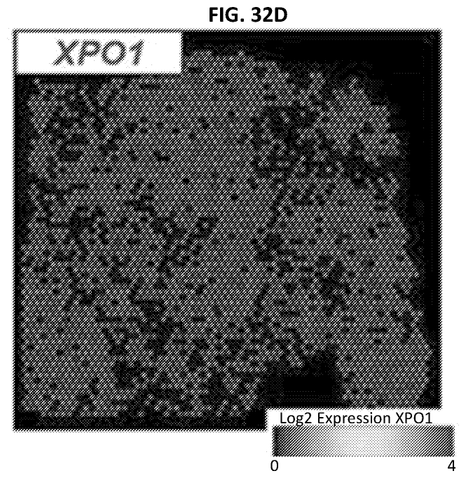

Using spatial gene expression of genes known to be expressed in ovarian cancer including, without limitation, BRCA1, BRCA2, MYC, TP53, PALB2, RAD51, MSH2, SCGB2A1, MKI67, PIK3CD, and CALML6, the abundance and/or spatial location of cancer cells in the ovarian cancer tissue section was identified. FIGS. 24A-24B show expression of BRCA1, BRCA2, MYC, TP53, PALB2, RAD51, and MSH2, and FIGS. 29A-29B show expression of SCGB2A1, MK167, PIK3CD, BRCA1, BRCA2, and CALML6. As shown in FIGS. 24A-24B and FIGS. 29A-29B, ovarian cancer genes expression was seen in each of the clusters and was were present throughout the tissue as expected (see also FIG. 9). In particular, MSH2 expression was seen in each cluster except cluster 4 (FIG. 24C), which is the cluster associated with B cells, and localized throughout the tissue but anti-correlated with CD45 staining, as expected (FIG. 24D). BRCA1 was not enriched in any of the clusters and overlay with Pan-CK and CD45 staining revealed localization mainly in cancerous regions (FIGS. 25A-25B, left panel). BRCA2 was enriched in cluster 7 and overlay with Pan-CK and CD45 staining revealed localization mainly in cancerous regions (FIGS. 25C-25D, right panel). In a parallel experiment assessing co-expression of cancer genes with either Pan-CK or CD45 (FIG. 32A), a number of clusters were identified. As shown in FIGS. 32B-32D, cluster 1 in this figure overlapped predominantly with Pan-CK tumor sections while Cluster 4 overlapped predominantly with CD45 stromal tissue sections. Gene expression levels are compared to expression in all other clusters. Each spot in FIGS. 32A-32D contained approximately 5,000 reads. In Cluster 1, PRKCI, VTCN1, MECOM, TOP2A (FIG. 32C), SHDH, XPO1 (FIG. 32D), TFRC, FUT8, SOX17, PBX1, EIF42, and WT1 were upregulated, indicating that each of these biomarkers can be used as a cancer biomarker. (e.g., an ovarian cancer biomarker).

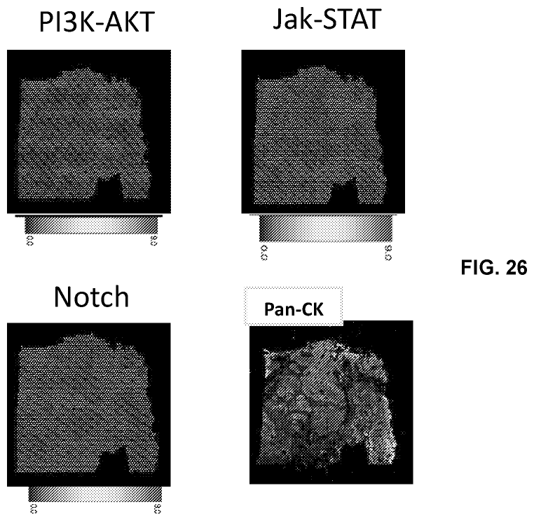

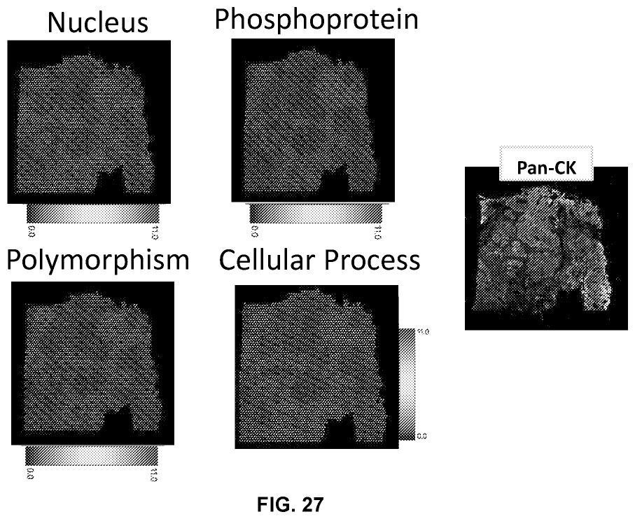

The methods described herein were also used for to stain for a panel of pan-cancer markers including analytes associated with PI3K-AKT signaling, Jak-STAT signaling, and NOTCH signaling (FIG. 26). Comparison to a Pan-CK stain of the tissue section shows enrichment of each of the pathways in the cancerous regions (FIG. 26). Gene expression patterns for pan-cancer panels associated with the nucleus, phosphoprotein, polymorphisms, and cell processes were also compared to Pan-CK staining (FIG. 27) to indicate the power of technology as a discover tool.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of analyzing immune cell infiltration in a cancer stromal region of a biological sample, the method comprising:
(a) identifying a cancerous region or an analyte associated with the cancerous region in the biological sample;
(b) identifying a stromal region or an analyte associated with the stromal region in the biological sample;
(c) identifying one or more immune cells or an analyte associated with an immune cell in one or more locations in the biological sample; and
(d) using (i) the identified cancerous region and stromal region or associated analytes thereof in the biological sample and (ii) the identified one or more immune cells or associated analytes thereof to analyze immune cell infiltration in the cancer stromal region of the biological sample.

2. The method of claim 1, wherein the identifying the cancerous region, the identifying the stromal region, and/or the identifying immune cells comprises:
(a) generating a dataset from the biological sample, wherein the dataset comprises one or more of:
(i) analyte data for a plurality of analytes captured from a plurality of spatial locations in the biological sample;
(ii) image data comprising images of the plurality of spatial locations of the biological sample; and
(iii) registration data linking the analyte data to the image data; and
(b) using the dataset to identify the cancerous region, the stromal region, and/or the immune cells in the biological sample.

3. The method of claim 2, wherein (b) comprises providing the dataset to a trained machine learning module, wherein the trained machine learning module is trained at least in part from training data comprising reference analyte datasets from one or more reference samples, wherein the one or more reference samples comprise (1) one or more reference cancerous regions, (2) one or more reference stromal regions, and (3) one or more reference immune cells.

4. The method of claim 3, wherein the abundance of immune cells is determined via the trained machine learning module.

5. The method of any one of the preceding claims, wherein the cancerous region comprises one or more of a benign tumor, a pre-metastatic tumor, a malignant tumor, and one or more inflammatory cells.

6. The method of any one of the preceding claims, wherein the stromal region comprises one or more of connective tissue, blood vessels, and inflammatory cells.

7. The method as in any one of the preceding claims, further comprising permeabilizing the biological sample.

8. The method of any one of the preceding claims, wherein the analyte associated with the cancerous region, an analyte associated with the stromal region, and/or an analyte associated with an immune cell is a nucleic acid.

9. The method of claim 8, wherein the nucleic acid is RNA.

10. The method of claim 9, wherein the RNA is an mRNA

11. The method of any one of the preceding claims, wherein the analyte associated with the cancerous region, the analyte associated with the stromal region, and/or the analyte associated with an immune cell is detected by the steps comprising:
contacting the biological sample with a substrate comprising a plurality of capture probes, wherein a capture probe of the plurality of capture probes comprises a spatial barcode and a capture domain;
hybridizing the analyte associated with the cancerous region, the analyte associated with the stromal region, and/or the analyte associated with an immune cell to the capture probe; and
determining (i) all or a part of a sequence corresponding to the analyte associated with the cancerous region, the analyte associated with the stromal region, and/or the analyte associated with an immune cell, or a complement thereof, and (ii) all or a part of a sequence corresponding to the spatial barcode, or a complement thereof, and using the determined sequence of (i) and (ii) to identify the abundance and/or spatial location of the analyte associated with the cancerous region, the analyte associated with the stromal region, and/or the analyte associated with an immune cell, or a complement thereof in the biological sample.

12. The method of claim 11, wherein the determining step comprises sequencing.

13. The method of any one of claims 1-7, wherein the analyte associated with the cancerous region, an analyte associated with the stromal region, and/or an analyte associated with an immune cell is a protein.

14. The method of claim 13, wherein the analyte associated with the cancerous region, the analyte associated with the stromal region, and/or the analyte associated with an immune cell is detected by the steps comprising:
attaching the biological sample with a plurality of analyte capture agents, wherein an analyte capture agent of the plurality of analyte capture agents comprises:
(i) an analyte binding moiety that binds specifically to the analyte associated with the cancerous region, the analyte associated with the stromal region, and/or the analyte associated with an immune cell;
(ii) an analyte binding moiety barcode; and
(iii) an analyte capture sequence, wherein the analyte capture sequence binds specifically to a capture domain;
contacting the biological sample with a substrate, wherein the substrate comprises a plurality of capture probes, wherein a capture probe of the plurality of capture probes comprises (i) the capture domain and (ii) a spatial barcode;
hybridizing the analyte associated with the cancerous region, the analyte associated with the stromal region, and/or the analyte associated with an immune cell to the capture probe; and
determining (i) all or a part of a sequence corresponding to the analyte associated with the cancerous region, the analyte associated with the stromal region, and/or the analyte associated with an immune cell, or a complement thereof, and (ii) all or a part of a sequence corresponding to the spatial barcode, or a complement thereof, and using the determined sequence of (i) and (ii) to identify the abundance and/or spatial location of the analyte associated with the cancerous region, the analyte associated with the stromal region, and/or the analyte associated with an immune cell, or a complement thereof in the biological sample.

15. The method of claim 14, wherein the determining step comprises:
sequencing (i) all or a part of a sequence corresponding to the analyte associated with the cancerous region, the analyte associated with the stromal region, and/or the analyte associated with an immune cell, or a complement thereof, and (ii) all or a part of a sequence corresponding to the spatial barcode, or a complement thereof, and using the determined sequence of (i) and (ii) to identify the abundance and/or spatial location of the analyte associated with the cancerous region, the analyte associated with the stromal region, and/or the analyte associated with an immune cell, or a complement thereof in the biological sample.

16. The method of claim 14 or 15, wherein the analyte binding moiety is an antibody or antigen-binding fragment thereof, a cell surface receptor binding molecule, a receptor ligand, a small molecule, a T-cell receptor engager, a B-cell receptor engager, a pro-body, an aptamer, a monobody, an affimer, or a darpin.

17. The method of any one of the preceding claims, wherein the analyte associated with the cancerous region, the analyte associated with the stromal region, and/or the analyte associated with an immune cell is detected using in situ sequencing.

18. The method of any one of the preceding claims, wherein the analyte associated with the cancerous region, the analyte associated with the stromal region, and/or the analyte associated with an immune cell is detected using an antibody.

19. The method of any one of one of the preceding claims, further comprising contacting the biological sample with one or more stains.

20. The method of claim 19, wherein the one or more stains comprises hematoxylin and eosin.

21. The method of claim 19 or 20, wherein the one or more stains comprise one or more optical labels.

22. The method of claim 21, wherein the one or more optical labels are selected from the group consisting of: fluorescent, radioactive, chemiluminescent, calorimetric, or colorimetric labels.

23. The method of any one of claims 19-22, further comprising identifying one or more cancerous regions in the biological sample using the one or more stains specific to a cancer marker.

24. The method of claim 23, wherein the cancer marker is pancytokeratin (Pan-CK).

25. The method of any one of claims 19-24, further comprising identifying one or more stromal regions within the one or more cancerous regions using the one or more stains specific to a stromal marker.

26. The method of claim 25, wherein the stromal marker is CD45.

27. The method of any one of claims 2-26, wherein the image data is generated by obtaining an image of the biological sample.

28. The method of claim 27, further comprising registering the image data to a spatial location.

29. The method of claim 27 or 28, further comprising identifying (1) the one or more cancerous regions and/or (2) the one or more stromal regions based on the image data.

30. The method of any one of claims 27-29, further comprising identifying the one or more immune cells based on the image data.

31. The method of any one of claims 2-30, further comprising identifying the one or more cancerous regions via the trained machine learning module.

32. The method of any one of claims 2-31, further comprising identifying the one or more stromal regions via the trained machine learning module.

33. The method of any one of claims 2-32, further comprising identifying the one or more immune cells via the trained machine learning module.

34. The method of any one of the preceding claims, wherein the analysis of immune cell infiltration in the cancer stromal region of the biological sample comprises determining abundance of immune cells in the cancer stromal region in the biological sample.

35. The method of any one of claim 11-34, wherein
identifying the one or more cancer regions comprises:
(i) obtaining an image and registering the image data to the spatial location,
(ii) using the spatial location of the determined sequences, or
(ii) obtaining an image and registering the image data to the spatial location, and using the spatial location of the determined sequences;
identifying the one or more stromal regions comprises:
(i) obtaining an image and registering the image data to the spatial location,
(ii) using the spatial location of the determined sequences, or
(iii) obtaining an image and registering the image data to the spatial location, and using the spatial location of the determined sequences; and
identifying the one or more immune cells or associated analytes thereof in one or more locations in the biological sample comprises:
(i) obtaining an image and registering the image data to the spatial location,
(ii) using the spatial location of the determined sequences, or
(iii) obtaining an image and registering the image data to the spatial location, and using the spatial location of the determined sequences.

36. The method of claim 34, wherein the abundance of immune cells in the cancer stromal region is determined as a percentage of cells in the cancer stroma area that are immune cells or a percentage of area of the cancer stroma that is occupied by immune cells.

37. The method of claim 36, wherein the abundance of immune cells in the cancer stromal region is determined using the spatial location of the determined sequence of the one or more cancerous regions, one or more stromal regions, and one or more immune cells.

38. The method of claim 37, wherein the using the spatial location of the determined sequences comprises determining the sequence using in situ sequencing.

39. The method of claim 36, wherein the abundance of immune cells in the cancer stromal region is determined using segmenting and
(i) obtaining an image and registering the image data to the spatial location,
(ii) using the spatial location of the determined sequences, or
(iii) obtaining an image and registering the image data to the spatial location, and using the spatial location of the determined sequences.

40. The method of any one of the preceding claims, wherein the determining comprises:
(a) identifying the amount of genes associated with immune infiltrating cells compared to known housekeepers normalized by number of cells per spatial location;
(b) identifying the ratio of one or more tumor infiltrating lymphocytes (TILs) to one or more tumor infiltrating B cells (TIBs); and/or
(c) calculating the abundance of tumor infiltrating immune cells in the biological sample based on the percentage of spatial locations comprising analytes associated with an immune infiltrating cells.

41. The method of any one of the preceding claims, wherein the identification of the one or more immune cells comprises segmenting immune cells from the image data.

42. The method of one of the preceding claims, further comprising determining a cancer prognosis based on the immune infiltration.

43. The method of one of the preceding claims, further comprising scoring or determining the severity of the cancer in the subject based on the immune infiltration score.

44. The method of one of the preceding claims, wherein the determining comprises identifying the ratio of one or more tumor infiltrating lymphocytes (TILs) to one or more tumor infiltrating B cells (TIBs) or one or more tumor infiltrating T cells to one or more tumor infiltrating B cells (TIBs).

45. The method of one of the preceding claims, further comprising administering a therapeutic treatment, wherein the therapeutic treatment comprises surgery, chemotherapeutic agents, growth inhibitory agents, cytotoxic agents, agents used in radiation therapy, anti-angiogenesis agents, cancer immunotherapeutic agents, apoptotic agents, antitubulin agents, or a combination thereof.

46. The method as in any one of the preceding claims, wherein the biological sample is obtained from a biopsy from a subject.

47. The method as in any one of the preceding claims, wherein the biological sample is obtained from a surgical excision from a subject.

48. The method of claim 46 or 47, wherein the biological sample is collected during an endoscopy or colonoscopy from a subject.

49. The method as in any one of the preceding claims, wherein the biological sample is a tissue section.

50. The method as in any one of the preceding claims, wherein the biological sample is a tissue section on a slide.

51. The method as in any one of the preceding claims, wherein the biological sample is a formalin-fixed, paraffin-embedded (FFPE) sample, a frozen sample, or a fresh sample.

52. The method as in any one of the preceding claims, wherein the biological sample is an FFPE sample.

53. The method of any one of the preceding claims, wherein the immune cells are selected from a B cell, a T cell, an NK cell, a monocyte, a macrophage, a neutrophil, a granulocyte, an innate lymphoid cell, or a dendritic cell or combinations thereof.

54. The method of any one of the preceding claims, wherein the analyte associated with the cancerous region is selected from an analyte from the AKT pathway, an analyte from the JAK-STAT pathway, and an analyte from the Notch pathway or combinations thereof.

55. The method of any one of the preceding claims, wherein the analyte associated with the cancerous region is selected from SCGB2A1, MKI67, BRCA1, BRCA2, PIKCD, CALML6, MYC, TP53, PALB2, RAD51, and MSH2 or combinations thereof.

56. The method of any one of the preceding claims, wherein the analyte associated with the cancerous region is selected from SCGB2A1, MKI67, BRCA1, BRCA2, PIK3CD, and CALML6 or combinations thereof.

57. The method of any one of the preceding claims, wherein the analyte associated with the cancerous region is selected from PRKCI, VTCN1, MECOM, TOP2A, SHDH, XPO1, TFRC, FUT8, SOX17, PBX1, EIF42, WTT, byproducts, precursors, and degradation products thereof, and any combination thereof.

58. The method of any one of the preceding claims, wherein the analyte associated with the cancerous region is selected from VTCN1, MECOM, TOP2A, XPO1, FUT8, SOX17, PBX1, EIF42, WTT, byproducts, precursors, and degradation products thereof, and any combination thereof.

59. The method of any one of the preceding claims, wherein the analyte associated with the cancerous region is TOP2A, and byproducts, precursors, and degradation products thereof.

60. The method of any one of the preceding claims, wherein the analyte associated with the cancerous region is XPO1, and byproducts, precursors, and degradation products thereof.

61. The method of any one of the preceding claims, wherein the analyte associated with the stromal region is selected from VIM, EPCAM, FAP, and CDH1.

62. The method of any one of the preceding claims, wherein the analyte associated with the stromal region is selected from FAP, VCAN, ACTA2, and PDGFRB.

63. The method of any one of the preceding claims, wherein the analyte associated with an immune cell is selected from BLK, CD19, FCRL2, MS4A1, KIAA0125, TNFRSF17, TCL1A, SPIB, PNOC, PTRPC, PRF1, GZMA, GZMB, NKG7, GZMH, KLRK1, KLRB1, KLRD1, CTSW, GNLY, CCL13, CD209, HSD11B1, LAG3, CD244, EOMES, PTGER4, CD68, CD84, CD163, MS4A4A, TPSB2, TPSAB1, CPA3, MS4A2, HDC, FPR1, SIGLEC5, CSF3R, FCAR, FCGR3B, CEACAM3, S100A12, KIR2DL3, KIR3DL1, KIR3DL2, IL21R, XCL1, XCL2, NCR1, CD6, CD3D, CD3E, SH2D1A, TRAT1, CD3G, TBX21, FOXP3, CD8A, CD8B, CD79A, CD79B, CD4, IGHA1, IGHG2, JCHAIN, IGKC, CD27, CD38, CD16, IL17RB, FANK1, CTLA4, MSR1, MRC1, NKG7, FCN1, TIGIT/LAG3.

64. The method as in any one of the preceding claims, wherein the one or more immune cells is selected from:
(i) a CD3⁺ and CD4⁺ T cell;
(ii) a CD3⁺ and CD8⁺ T cell;
(iii) a regulatory T cell comprising one or more of: CD4, Foxp3, IL17RB, CTLA4, FANK1, HAVCR1, CD25, CTLA-4, GITR, LAG-3, and CD127;
(iv) a TH1 cell comprising one or more of: CD4, CD3D, S100A4, IL7R, and IFNG;
(v) a TH2 cell comprising one or more of: CD4, IL7R, ICOS, CTLA4, TNFRSF4, and TNFRS18;
(vi) a TH17 cell comprising one or more of: CD4, CD3D, IL17A, GZMA, and S100A4;
(vii) a cytotoxic T cell comprising one or more of: CD8, CD3D, S100A4, IFNG, GZMB, GZMA, and IL2RB;
(viii) a plasma cell comprising: one or more JCHAIN, MZB1, IGHA1, IGHG1, and IGKC;
(ix) a monocyte comprising CD14⁺ CD16⁻;
(x) a monocyte comprising CD14⁻ CD16⁺; and
(xi) a natural killer cell comprising NKG7.

65. The method of any one of the preceding claims, wherein the immune infiltrating cells is a tumor infiltrating B cell (TIB).

66. The method of claim 65, wherein the TIB is selected from:
(i) a plasma cell comprising one or more of MZB1, IGLL5, IGHA1, IGHG1, JCHAIN, IGKC, IGHA2, IGLC2, IGLV3-1, and IGLV2-14;
(ii) an Ig⁺ B cells comprising one or more of IGHV3-74, SOCS3, JCHAIN, and SPARC;
(iii) an activated B cell comprising: CD79B, HMGB2, HMGB1, HMGN1, and RGS13;
(iv) a B cell comprising one or more of: MEF2B, RGS13, and MS4A1; and
(v) a B cell comprising CD79A and CD79B.

67. A method of determining immune cell infiltration in a biological sample comprising one or more cancerous regions and one or more stromal regions in a subject, the method comprising:
(a) generating a dataset from the biological sample obtained from the subject, wherein the dataset comprises:
(i) analyte data for a plurality of analytes captured from a plurality of spatial locations of the biological sample, wherein an analyte in the plurality of analytes is an analyte associated with the cancerous region, an analyte associated with the stromal region, and/or an analyte associated with an immune cell;
(b) providing the dataset to a trained machine learning module, wherein the trained machine learning module comprises reference analyte datasets from one or more reference samples, wherein the one or more reference samples comprises (i) a cancerous region from one or more cancerous regions, (2) a stromal region from one or more stromal regions, and (3) an immune cells from one or more immune cells; and
(c) determining, via the trained machine learning module, the immune cell infiltration in the biological sample obtained from the subject.

68. A method of determining immune cell infiltration in a biological sample comprising one or more cancerous regions and one or more stromal regions, the method comprising:
(a) generating a dataset from the biological sample obtained from a subject, wherein the dataset comprises:
(i) analyte data for a plurality of analytes captured from a plurality of spatial locations of the biological sample, wherein an analyte in the plurality of analytes is an analyte associated with the cancerous region, an analyte associated with the stromal region, and/or an analyte associated with an immune cell;
(ii) image data comprising images of the plurality of spatial locations of the biological sample; and
(iii) registration data linking the analyte data to the image data;
(b) providing the dataset to a trained machine learning module, wherein the trained machine learning module comprises reference analyte datasets from one or more reference samples, wherein the one or more reference samples comprises (i) a cancerous region from one or more cancerous regions, (2) a stromal region from one or more stromal regions, and (3) an immune cells from one or more immune cells; and
(c) determining, via the trained machine learning module, the immune cell infiltration in the biological sample.

69. The method of claim 67 or 68, wherein the trained machine learning module is at least one of a supervised learning module, a semisupervised learning module, an unsupervised learning module, a regression analysis module, a reinforcement learning module, a self-learning module, a feature learning module, a sparse dictionary learning module, an anomaly detection module, a generative adversarial network, a convolutional neural network, or an association rules module.

70. The method of any one of claims 67-69, wherein generating the dataset comprises:
contacting a biological sample having cancer with a substrate comprising a plurality of capture probes, wherein in the biological sample comprises (1) one or more cancerous regions, (2) one or more stromal regions, and (3) one or more tumor infiltrating immune cells, and wherein a capture probe of the plurality of capture probes comprises a spatial barcode and a capture domain;
attaching an analyte from the biological sample to the capture probe;
determining (i) all or a part of a sequence corresponding to the analyte, or a complement thereof, and (ii) all or a part of a sequence corresponding to the spatial barcode, or a complement thereof, and using the determined sequence of (i) and (ii) to identify the spatial location and abundance of the analyte in the biological sample; and
identifying a spatial location as being part of a cluster based on the determined sequences corresponding to the analytes at the spatial location and using the clusters to analyze immune cell infiltration in the cancer stroma of the subject having cancer.

71. The method of claim 70, wherein a cluster one or more immune cells is identified using one of the methods selected from: nonlinear dimensionality reduction, t-distributed stochastic neighbor embedding (t-SNE), global t-distributed stochastic neighbor embedding (g-SNE), and uniform manifold approximation and projection (UMAP).

72. The method of any one of claims 67-71, wherein generating the dataset comprises:
attaching the biological sample with a plurality of analyte capture agents, wherein an analyte capture agent of the plurality of analyte capture agents comprises:
(i) an analyte binding moiety that binds specifically to the analyte associated with the cancerous region, the analyte associated with the stromal region, and/or the analyte associated with an immune cell;
(ii) an analyte binding moiety barcode; and
(iii) an analyte capture sequence, wherein the analyte capture sequence binds specifically to a capture domain;
contacting the biological sample with a substrate, wherein the substrate comprises a plurality of capture probes, wherein a capture probe of the plurality of capture probes comprises (i) the capture domain and (ii) a spatial barcode;
hybridizing the analyte associated with the cancerous region, the analyte associated with the stromal region, and/or the analyte associated with an immune cell to the capture probe; and
determining (i) all or a part of a sequence corresponding to the analyte associated with the cancerous region, the analyte associated with the stromal region, and/or the analyte associated with an immune cell, or a complement thereof, and (ii) all or a part of a sequence corresponding to the spatial barcode, or a complement thereof, and using the determined sequence of (i) and (ii) to identify the abundance and/or spatial location of the analyte associated with the cancerous region, the analyte associated with the stromal region, and/or the analyte associated with an immune cell, or a complement thereof in the biological sample.

73. The method of any one of claims 66-71, wherein the analyte data is generated using in situ sequencing.

74. A kit comprising:
(a) an antibody that specifically binds to an antigen on an infiltrating immune cell;
(b) a substrate comprising a plurality of capture probe, wherein an capture probe of the plurality of capture probes comprises a capture domain; and
(c) instructions for performing the method of any one of claims 1-72.

75. A kit comprising:
(a) an antibody that specifically binds to an antigen on an infiltrating immune cell;
(b) a second antibody that specifically binds to an antigen on a stromal cell;
(c) a substrate comprising a plurality of capture probe, wherein an capture probe of the plurality of capture probes comprises a capture domain; and
(d) instructions for performing the method of claim 1-72.

76. A computer implemented method comprising:
(a) generating a dataset of a plurality of biological samples, wherein the dataset comprises, for each biological sample of the plurality of biological samples:
(i) analyte data for a plurality of analytes captured at a plurality of spatial locations of a reference biological sample;
(ii) image data of the reference biological sample; and
(iii) registration data of the imaged data linking to the analyte data according to the spatial locations of the reference biological sample;
wherein the reference biological sample comprises
(1) one or more cancerous regions in the reference biological sample,
(2) one or more stromal regions within the one or more cancerous regions, and
(3) a plurality of tumor infiltrating lymphocytes (TILs);
(b) training a machine learning module with the dataset, thereby generating a trained machine learning module; and
(c) determining immune cell infiltration in a biological sample via the trained machine learning module.

77. A system comprising:
(a) a storage element operable to store a dataset of a plurality of biological samples, wherein the dataset comprises: analyte data for a plurality of analytes captured at a plurality of spatial locations of a reference biological sample; image data of the biological sample; and registration data of the imaged data linking to the analyte data according to the spatial locations of the reference biological sample; wherein the biological sample comprises (1) one or more cancerous regions in the reference biological sample, (2) one or more stromal regions within the one or more cancerous regions, and (3) the a plurality of tumor infiltrating lymphocytes (TILs); and
(b) a processor operable to process the dataset through a machine learning module to train the machine learning module, to determine immune cell infiltration in a biological sample.
